(19) United States
(12) Patent Application Publication
(10) Pub. No.: US 2025/0215517 A1
(43) Pub. Date: Jul. 3, 2025

(54) METHOD FOR DETECTING TARGET NUCLEIC ACIDS OF AT LEAST NINE HPV TYPES IN SAMPLE

(71) Applicant: SEEGENE, INC., Seoul (KR)

(72) Inventors: Ju-Hee BAE, Seoul (KR); Byung Ha LEE, Seoul (KR); Sodam JIN, Gyeonggi-do (KR); Hyungjun JANG, Seoul (KR)

(21) Appl. No.: 18/852,009

(22) PCT Filed: Apr. 5, 2023
(86) PCT No.: PCT/KR2023/004623

§ 371 Date: Sep. 27, 2024

(30) Foreign Application Priority Data
Apr. 8, 2022 (KR) 10-2022-0044212
Apr. 8, 2022 (KR) 10-2022-0044213
Apr. 8, 2022 (KR) 10-2022-0044214
Apr. 8, 2022 (KR) 10-2022-0044215
Apr. 8, 2022 (KR) 10-2022-0044216

(51) Int. Cl. C12Q 1/70; C12Q 1/6851; C12Q 1/686

(52) U.S. Cl. CPC C12Q 1/701; C12Q 1/6851; C12Q 1/686; C12Q 2600/16

(57) ABSTRACT

The present method allows for detection of at least nine HPV types in a real-time manner in one reaction vessel by analyzing signals measured at three temperatures using at least three different types of fluorescent labels. In particular, the method of the present invention can detect at least nine target nucleic acids in a real-time manner without performing a melting analysis.

### TECHNICAL FIELD

The present disclosure relates to a method of detecting target nucleic acids of at least nine HPV types in a sample.

### BACKGROUND ART

Human papillomaviruses (HPVs), which belong to the Papillomaviridae family, constitute a group of small nonenveloped double-stranded DNA viruses. HPV has a small genome that only encodes a few proteins, and it is also responsible for 5% of all human cancers, including cervical, vaginal, vulvar, penile, anal, and oropharyngeal cancers. HPV types may be classified as high- and low-risk genotypes (HR-HPVs and LR-HPVs, respectively) according to their oncogenic potential.

HPV genome is functionally divided into three regions: early region (E region), late region (L region) and non-coding region (NCR). The E region can be further divided into seven open reading frames (E1 to E7) that mainly encode proteins involved in viral replication, transcription, regulation, and cell transformation. The L region can be divided into L1 and L2 encoding the main capsid protein and the subcapsid protein, respectively.

To date, more than 200 HPV types have been identified, and ˜40 HPV genotypes have been detected in the female genital tract. HPV16 and HPV18 are well known as oncogenic genotypes; additionally, HPV31, HPV33, HPV35, HPV39, HPV45, HPV51, HPV52, HPV56, HPV58, HPV59, HPV68, HPV69, and HPV 82 are also closely associated with cervical cancer. Therefore, all of these genotypes are classified as “highrisk” HPV. Meanwhile, “low-risk” genotypes, including HPV6, HPV11, HPV42, HPV43, and HPV44 are the causative agents for benign or low-grade changes in cervical cells, such as genital warts.

In 1995, the International Agency for Research on Cancer (IARC) symposium concluded that HPV infection was the most important cause of cervical cancer. Certain types of HPV can also cause anal cancer, oropharyngeal cancer, vulvar cancer, vaginal cancer, and penile cancer. Most HPV infections are asymptomatic and are automatically eliminated by the immune system within 1-2 years. Therefore, regular HPV testing can effectively prevent the occurrence of related cancers.

For detection of HPV, real-time detection methods capable of monitoring target amplification in a real-time manner have been widely used. The real-time detection methods typically use labeled probes or primers that are specifically hybridized with the target nucleic acid. Examples of methods using hybridization between labeled probes and target nucleic acids include, without limitation, molecular beacon methods using dual labeled probes having hairpin structures (Tyagi et al., Nature Biotechnology v. 14 Mar. 1996), HyBeacon methods (French D J et al., Mol. Cell probes, (6): 363-374 (2001)), a hybridization probe method using two probes each labeled with a donor or an acceptor (Bernad et al., 147-148 Clin Chem 2000; 46) and a Lux method using a single-labeled oligonucleotide (U.S. Pat. No. 7,537,886). The TaqMan method (U.S. Pat. Nos. 5,210,015 and 5,538,848) using dual labeled probes and cleavage of the probes by 5′-nuclease activity of DNA polymerase is widely used in the art. Examples of methods using labeled primers include, without limitation, the Sunrise primer method (Nazarenko et al., 2516-2521 Nucleic Acids Research, 1997, v. 25 no. 12, and U.S. Pat. No. 6,117,635), the Scorpion primer method (Whitcombe et al., 804-807, Nature Biotechnology v. 17 Aug. 1999 and U.S. Pat. No. 6,326,145) and the TSG primer method (WO 2011-078441).

As alternative approaches, real-time detection methods using duplexes formed dependent on the presence of the target nucleic acid have been proposed: Invader assay (U.S. Pat. Nos. 5,691,142, 6,358,691 and 6,194,149), PTO cleavage and extension (PTOCE) method (WO 2012/096523), PTO cleavage and extension-dependent signaling oligonucleotide hybridization (PCE-SH) method (WO 2013/115442), PTO cleavage and extension-dependent non-hybridization (PCE-NH) method (WO 2014/104818).

The conventional real-time detection techniques described above can detect only one target nucleic acid using one label, which limits the number of target nucleic acids that can be simultaneously detected in one reaction to the number of available labels (e.g., or less).

Melting analysis can be used to detect multiple target nucleic acids using a single label, but it has drawbacks in that it takes longer to perform than real-time techniques, and as the number of target nucleic acids increases, the design of probes with different Tₘ values becomes increasingly difficult.

As such, conventional real-time detection techniques or melting analyses are limited when multiple target nucleic acids are to be detected, such as HPV genotyping.

Therefore, there is a need in the art to develop a real-time detection method capable of simultaneously detecting multiple HPV types in one reaction, despite the use of a limited labels.

Throughout this application, various patents and publications are referenced and citations are provided in parentheses. The disclosure of these patents and publications in their entirety are hereby incorporated by references into this application in order to more fully describe this invention and the state of the art to which this invention pertains.

### DISCLOSURE OF INVENTION

### Technical Problem

The present inventors have sought to develop a real-time amplification method for HPV genotyping in one reaction vessel. As a result, the present inventors have developed a method of distinguishably detecting three HPV types per a fluorescent label, thereby simultaneously detecting target nucleic acids of at least nine HPV types using at least three fluorescent labels.

Thus, it is an object of the present invention to provide a method and a kit for detecting target nucleic acids of at least nine HPV types in a sample.

It is another object of the present invention to provide a computer readable storage medium containing instructions to configure a processor to perform a method of detecting target nucleic acids of at least nine human papillomavirus (HPV) types in a sample.

It is still another object of the present invention to provide a device for detecting target nucleic acids of at least nine human papillomavirus (HPV) types in a sample.

It is still another object of the present invention to provide a computer program to be stored on a computer readable storage medium to configure a processor to perform a method of detecting target nucleic acids of at least nine human papillomavirus (HPV) types in a sample.

Other objects and advantages of the present invention will become apparent from the detailed description to follow taken in conjugation with the appended claims and drawings.

### Solution to Problem

In an aspect of the invention, the present disclosure provides a method for detecting target nucleic acids of at least nine human papillomavirus (HPV) types in a sample, comprising:
(a) incubating the sample in a single reaction vessel with at least nine oligonucleotide sets to amplify or detect target nucleic acids of at least nine HPV types,
wherein each of the at least nine oligonucleotide sets comprises:
(i) an amplifying oligonucleotide, which serves to amplify a target nucleic acid of an HPV type of interest; and
(ii) a signaling oligonucleotide having a fluorescent label linked thereto, which serves to generate a signal in the presence of a target nucleic acid of an HPV type of interest,
wherein the amplifying oligonucleotide and the signaling oligonucleotide are identical to or different from each other,
wherein the fluorescent labels included in the at least nine oligonucleotide sets are of at least three different types, which are distinguishable by at least three different detection channels, and three of the at least nine oligonucleotide sets comprise any one of the at least three different types of the fluorescent labels,
wherein for three oligonucleotide sets comprising any one of the at least three different types of the fluorescent labels, one oligonucleotide set is designed to generate a signal at a first detection temperature in the presence of a target nucleic acid of a corresponding HPV type, another oligonucleotide set is designed to generate a signal at the first detection temperature and a second detection temperature in the presence of a target nucleic acid of another corresponding HPV type, and the other oligonucleotide set is designed to generate a signal at the first detection temperature, the second detection temperature and a third detection temperature in the presence of a target nucleic acid of the other corresponding HPV type;
(b) measuring signals at the first detection temperature, the second detection temperature, and the third detection temperature using each of the at least three detection channels; and
(c) determining the presence of target nucleic acids of the at least nine HPV types by the signals measured at the first detection temperature, the second detection temperature, and the third detection temperature using each of the at least three detection channels.

In an embodiment, the incubating is performed by real-time PCR in which Ct values are provided for each of target nucleic acids of the at least nine HPV.

In an embodiment, the at least nine oligonucleotide sets comprise oligonucleotide sets specific for HPV types selected from the group consisting of HPV 16, 18, 31, 33, 35, 39, 45, 51, 52, 56, 58, 59, 66, and 68.

In another embodiment, the at least nine oligonucleotide sets comprise oligonucleotide sets specific for HPV types selected from the group consisting of HPV 6, 11, 26, 40, 42, 43, 44, 53, 54, 61, 69, 70, 73, and 82.

In an embodiment, the at least nine oligonucleotide sets are fifteen (15) oligonucleotide sets.

In an embodiment, the fifteen oligonucleotide sets consist of oligonucleotide sets specific for HPV 16, 18, 31, 33, 35, 39, 45, 51, 52, 56, 58, 59, 66, 68, and an internal control.

In another embodiment, the fifteen oligonucleotide sets consist of oligonucleotide sets specific for HPV 6, 11, 26, 40, 42, 43, 44, 53, 54, 61, 69, 70, 73, 82, and an internal control.

In an embodiment, the total number of oligonucleotides in the at least nine oligonucleotide sets is at least thirty-six (36).

In another embodiment, the total number of oligonucleotides in the at least nine oligonucleotide sets is at least forty-five (45).

In an embodiment, one or more of the at least nine oligonucleotide sets generate a signal by formation or dissociation of a duplex from the signaling oligonucleotide, which occurs dependently on the presence of a target nucleic acid.

In another embodiment, one or more of the at least nine oligonucleotide sets generate a signal by cleavage of a mediation oligonucleotide hybridized to the target nucleic acid and then formation or dissociation of a duplex between the cleavage product and the signaling oligonucleotide, which occurs dependently on the presence of the target nucleic acid.

In still another embodiment, one or more of the at least nine oligonucleotide sets generate a signal by cleavage of the signaling oligonucleotide hybridized to the target nucleic acid and then dissociation of a duplex, which occurs dependently on the presence of the target nucleic acid.

In a further embodiment, one or more of the at least nine oligonucleotide sets generate a signal by cleavage of the signaling oligonucleotide hybridized to the target nucleic acid and then dissociation of a duplex, which occurs dependently on the presence of the target nucleic acid.

In an embodiment, the total concentration of the amplifying oligonucleotides in the reaction vessel is 15 to 60 pmole/μL.

In an embodiment, the total reaction volume in the reaction vessel is 10 to 30 μL.

In an embodiment, the total concentration of the signaling oligonucleotides in the reaction vessel is 8 to 35 pmole/μL.

In an embodiment, the first detection temperature is selected from 57 to 63° C., the second detection temperature is selected from 69 to 75° C., and the third detection temperature is selected from 80 to 86° C.

In another embodiment, the first detection temperature is selected from 80 to 86° C., the second detection temperature is selected from 69 to 75° C., and the third detection temperature is selected from 57 to 63° C.

In an embodiment, the step (c) is performed using signals measured at the first detection temperature, the second detection temperature, and the third detection temperature for each of the at least three detection channels, and three reference values.

In an embodiment, the step (c) comprises extracting three signals, a signal generated only by one oligonucleotide set, a signal generated only by another oligonucleotide set, and a signal generated only by the other oligonucleotide set, from signals measured at the first detection temperature, the second detection temperature and the third detection temperature for each of the at least three detection channels.

In an embodiment, the method is used for HPV genotyping.

In another aspect of the invention, the present disclosure provides a kit for detecting target nucleic acids of at least nine human papillomavirus (HPV) types in a sample, comprising:
(a) at least nine oligonucleotide sets to amplify or detect target nucleic acids of at least nine HPV types,
wherein each of the at least nine oligonucleotide sets comprises:
(i) an amplifying oligonucleotide, which serves to amplify a target nucleic acid of an HPV type of interest; and
(ii) a signaling oligonucleotide having a fluorescent label linked thereto, which serves to generate a signal in the presence of a target nucleic acid of an HPV type of interest,
wherein the amplifying oligonucleotide and the signaling oligonucleotide are identical to or different from each other,
wherein the fluorescent labels included in the at least nine oligonucleotide sets are of at least three different types, which are distinguishable by at least three different detection channels, and three of the at least nine oligonucleotide sets comprise any one of the at least three different types of the fluorescent labels,
wherein for three oligonucleotide sets comprising any one of the at least three different types of the fluorescent labels, one oligonucleotide set is designed to generate a signal at a first detection temperature in the presence of a target nucleic acid of a corresponding HPV type, another oligonucleotide set is designed to generate a signal at the first detection temperature and a second detection temperature in the presence of a target nucleic acid of another corresponding HPV type, and the other oligonucleotide set is designed to generate a signal at the first detection temperature, the second detection temperature and a third detection temperature in the presence of a target nucleic acid of the other corresponding HPV type;
(b) an instruction that describes the method of the present disclosure.

In another aspect of the invention, the present disclosure provides a computer readable storage medium containing instructions to configure a processor to perform a method of detecting target nucleic acids of at least nine human papillomavirus (HPV) types in a sample, the method comprising:
(a) receiving signals measured at a first detection temperature, a second detection temperature, and a third detection temperature using each of at least three detection channels,
wherein the signals are obtained by incubating the sample in a single reaction vessel with at least nine oligonucleotide sets to amplify or detect target nucleic acids of at least nine HPV types and measuring signals at the first detection temperature, the second detection temperature, and the third detection temperature using each of the at least three detection channels,
wherein each of the at least nine oligonucleotide sets comprises:
(i) an amplifying oligonucleotide, which serves to amplify a target nucleic acid of an HPV type of interest; and
(ii) a signaling oligonucleotide having a fluorescent label linked thereto, which serves to generate a signal in the presence of a target nucleic acid of an HPV type of interest,
wherein the amplifying oligonucleotide and the signaling oligonucleotide are identical to or different from each other,
wherein the fluorescent labels included in the at least nine oligonucleotide sets are of at least three different types, which are distinguishable by at least three different detection channels, and three of the at least nine oligonucleotide sets comprise any one of the at least three different types of the fluorescent labels,
wherein for three oligonucleotide sets comprising any one of the at least three different types of the fluorescent labels, one oligonucleotide set is designed to generate a signal at a first detection temperature in the presence of a target nucleic acid of a corresponding HPV type, another oligonucleotide set is designed to generate a signal at the first detection temperature and a second detection temperature in the presence of a target nucleic acid of another corresponding HPV type, and the other oligonucleotide set is designed to generate a signal at the first detection temperature, the second detection temperature and a third detection temperature in the presence of a target nucleic acid of the other corresponding HPV type;
(b) determining the presence of target nucleic acids of the at least nine HPV types by signals measured at the first detection temperature, the second detection temperature, and the third detection temperature using each of the at least three detection channels.

In another aspect of the invention, the present disclosure provides a device for detecting target nucleic acids of at least nine human papillomavirus (HPV) types in a sample, comprising (a) a computer processor and (b) the computer readable storage medium described above coupled to the computer processor.

In another aspect of the invention, the present disclosure provides a computer program to be stored on a computer readable storage medium to configure a processor to perform a method of detecting target nucleic acids of at least nine human papillomavirus (HPV) types in a sample, the method comprising:
(a) receiving signals measured at a first detection temperature, a second detection temperature, and a third detection temperature using each of at least three detection channels,
wherein the signals are obtained by incubating the sample in a single reaction vessel with at least nine oligonucleotide sets that are designed to amplify or detect target nucleic acids of at least nine HPV types and measuring signals at the first detection temperature, the second detection temperature, and the third detection temperature using each of the at least three detection channels,
wherein each of the at least nine oligonucleotide sets comprises:
(i) an amplifying oligonucleotide, which serves to amplify a target nucleic acid of an HPV type of interest; and
(ii) a signaling oligonucleotide having a fluorescent label linked thereto, which serves to generate a signal in the presence of a target nucleic acid of an HPV type of interest,
wherein the amplifying oligonucleotide and the signaling oligonucleotide are identical to or different from each other,
wherein the fluorescent labels included in the at least nine oligonucleotide sets are of at least three different types, which are distinguishable by at least three different detection channels, and three of the at least nine oligonucleotide sets comprise any one of the at least three different types of the fluorescent labels,
wherein for three oligonucleotide sets comprising any one of the at least three different types of the fluorescent labels, one oligonucleotide set is designed to generate a signal at a first detection temperature in the presence of a target nucleic acid of a corresponding HPV type, another oligonucleotide set is designed to generate a signal at the first detection temperature and a second detection temperature in the presence of a target nucleic acid of another corresponding HPV type, and the other oligonucleotide set is designed to generate a signal at the first detection temperature, the second detection temperature and a third detection temperature in the presence of a target nucleic acid of the other corresponding HPV type;
(b) determining the presence of target nucleic acids of the at least nine HPV types by signals measured at the first detection temperature, the second detection temperature, and the third detection temperature using each of the at least three detection channels.

### Advantageous Effects of Invention

The present method allows for detection of at least nine HPV types in a real-time manner in one reaction vessel by analyzing signals measured at three temperatures using at least three different types of fluorescent labels. Conventional real-time PCR is disadvantageous in that the number of target nucleic acids detectable at the same time is limited by the number of available labels (e.g., 5 or less), and conventional melting analysis requires an additional melting process after target amplification. In contrast, the method of the present invention can detect at least nine target nucleic acids in a real-time manner without performing a melting analysis.

Further, the method of the present invention can genotype a number of HPV viruses including high-risk and low-risk HPV types in one reaction, and thus is cost-effective and time-saving.

### BRIEF DESCRIPTION OF DRAWINGS

FIG. 1 shows the results of incubating a target nucleic acid of HPV 66 having a low detection temperature (60° C.), a target nucleic acid of HPV 45 having a middle detection temperature (72° C.), and a target nucleic acid of HPV 58 having a high detection temperature (83° C.) with three oligonucleotide sets comprising FAM as a fluorescent label so as to amplify each target nucleic acid, and then measuring signals at 60° C. (top), 72° C. (middle) and 83° C. (bottom). The signal for each target nucleic acid was generated by the PTOCE method (see WO 2012/096523). As shown in the figure, since the signal measured at 60° C. includes mixed signals for the target nucleic acids of HPV 66, HPV 45 and HPV 58, and the signal measured at 72° C. includes mixed signals for the target nucleic acids of HPV 66 and HPV 45, and the signal measured at 83° C. includes a signal for the target nucleic acid of HPV 58 only, the intensity of the signal measured at 60° C. is greater than that of the signal measured at 72° C., which is greater than that of the signal measured at 83° C.

FIG. 2 shows extracted signals for HPV 66 (top), HPV 45 (middle) and HPV 58 (bottom), respectively, from the signals measured at the three detection temperatures as shown in FIG. 1.

FIG. 3 shows the results of incubating a target nucleic acid of HPV 51 having a low detection temperature (60° C.), a target nucleic acid of HPV 59 having a middle detection temperature (72° C.), and a target nucleic acid of HPV 16 having a high detection temperature (83° C.) with three oligonucleotide sets comprising CAL Fluor Orange 560 as a fluorescent label so as to amplify each target nucleic acid, and then measuring signals at 60° C. (top), 72° C. (middle) and 83° C. (bottom). The signal for each target nucleic acid was generated by the PTOCE method (see WO 2012/096523). As shown in the figure, since the signal measured at 60° C. includes mixed signals for the target nucleic acids of HPV 51, HPV 59 and HPV 16, and the signal measured at 72° C. includes mixed signals for the target nucleic acids of HPV 51 and HPV 59, and the signal measured at 83° C. includes a signal for the target nucleic acid of HPV 16 only, the intensity of the signal measured at 60° C. is greater than that of the signal measured at 72° C., which is greater than that of the signal measured at 83° C.

FIG. 4 shows extracted signals for HPV 51 (top), HPV 59 (middle) and HPV 16 (bottom), respectively, from the signals measured at the three detection temperatures as shown in FIG. 3.

FIG. 5 shows the results of incubating a target nucleic acid of HPV 33 having a low detection temperature (60° C.), a target nucleic acid of HPV 39 having a middle detection temperature (72° C.), and a target nucleic acid of HPV 52 having a high detection temperature (83° C.) with three oligonucleotide sets comprising CAL Fluor Red 610 as a fluorescent label so as to amplify each target nucleic acid, and then measuring signals at 60° C. (top), 72° C. (central) and 83° C. (bottom). The signal for each target nucleic acid was generated by the PTOCE method (see WO 2012/096523). As shown in the figure, since the signal measured at 60° C. includes mixed signals for the target nucleic acids of HPV 33, HPV 39 and HPV 52, and the signal measured at 72° C. includes mixed signals for the target nucleic acids of HPV 33 and HPV 39, and the signal measured at 83° C. includes a signal for the target nucleic acid of HPV 39 only, the intensity of the signal measured at 60° C. is greater than that of the signal measured at 72° C., which is greater than that of the signal measured at 83° C.

FIG. 6 shows extracted signals for HPV 33 (top), HPV 39 (middle) and HPV 52 (bottom), respectively, from the signals measured at the three detection temperatures as shown in FIG. 5.

FIG. 7 shows the results of incubating an internal control (IC) having a low detection temperature (60° C.), a target nucleic acid of HPV 35 having a middle detection temperature (72° C.), and a target nucleic acid of HPV 18 having a high detection temperature (83° C.) with three oligonucleotide sets comprising Quasar 670 as a fluorescent label so as to amplify each target nucleic acid, and then measuring signals at 60° C. (top), 72° C. (middle) and 83° C. (bottom). The signal for each target nucleic acid was generated by the PTOCE method (see WO 2012/096523). As shown in the figure above, since the signal measured at 60° C. includes mixed signals for the target nucleic acids of the internal control, HPV 35 and HPV 18, and the signal measured at 72° C. includes mixed signals for the target nucleic acids of HPV 35 and HPV 18, and the signal measured at 83° C. includes a signal for the target nucleic acid of HPV 18 only, the intensity of the signal measured at 60° C. is greater than that of the signal measured at 72° C., which is greater than that of the signal measured at 83° C.

FIG. 8 shows extracted signals for the internal control (top), HPV 35 (middle) and HPV 18 (bottom), respectively, from the signals measured at the three detection temperatures as shown in FIG. 7.

FIG. 9 shows the results of incubating a target nucleic acid of HPV 56 having a low detection temperature (60° C.), a target nucleic acid of HPV 68 having a middle detection temperature (72° C.), and a target nucleic acid of HPV 31 having a high detection temperature (83° C.) with three oligonucleotide sets comprising Quasar 705 as a fluorescent label so as to amplify each target nucleic acid, and then measuring signals at 60° C. (top), 72° C. (middle) and 83° C. (bottom). The signal for each target nucleic acid was generated by the PTOCE method (see WO 2012/096523). As shown in the figure, since the signal measured at 60° C. includes mixed signals for the target nucleic acids of HPV 56, HPV 68 and HPV 31, and the signal measured at 72° C. includes mixed signals for the target nucleic acids of HPV 68 and HPV 31, and the signal measured at 83° C. includes a signal for the target nucleic acid of HPV 31, the intensity of the signal measured at 60° C. is greater than that of the signal measured at 72° C., which is greater than that of the signal measured at 83° C.

FIG. 10 shows extracted signals for HPV 56 (top), HPV 68 (middle) and HPV 31 (bottom), respectively, from the signals measured at the three detection temperatures as shown in FIG. 9.

### BEST MODE FOR CARRYING OUT THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

The key feature of the present invention is the simultaneous detection of target nucleic acids of at least nine HPV types in one real-time amplification reaction. Conventional real-time amplification reactions have made it impossible to simultaneously detect more than five target nucleic acids due to the limitation of the number of available fluorescent labels. In contrast, the method of the present invention can simultaneously detect at least nine HPV types using at least three different types of fluorescent labels in a real-time manner without performing a melting analysis.

### I. Method for Detecting Target Nucleic Acids

In one aspect of the invention, there is provided a method for detecting target nucleic acids of at least nine human papillomavirus (HPV) types in a sample, comprising:
(a) incubating the sample in a single reaction vessel with at least nine oligonucleotide sets to amplify or detect target nucleic acids of at least nine HPV types,
wherein each of the at least nine oligonucleotide sets comprises:
(i) an amplifying oligonucleotide, which serves to amplify a target nucleic acid of an HPV type of interest; and
(ii) a signaling oligonucleotide having a fluorescent label linked thereto, which serves to generate a signal in the presence of a target nucleic acid of an HPV type of interest,
wherein the amplifying oligonucleotide and the signaling oligonucleotide are identical to or different from each other,
wherein the fluorescent labels included in the at least nine oligonucleotide sets are of at least three different types, which are distinguishable by at least three different detection channels, and three of the at least nine oligonucleotide sets comprise any one of the at least three different types of the fluorescent labels,
wherein for three oligonucleotide sets comprising any one of the at least three different types of the fluorescent labels, one oligonucleotide set is designed to generate a signal at a first detection temperature in the presence of a target nucleic acid of a corresponding HPV type, another oligonucleotide set is designed to generate a signal at the first detection temperature and a second detection temperature in the presence of a target nucleic acid of another corresponding HPV type, and the other oligonucleotide set is designed to generate a signal at the first detection temperature, the second detection temperature and a third detection temperature in the presence of a target nucleic acid of the other corresponding HPV type;
(b) measuring signals at the first detection temperature, the second detection temperature, and the third detection temperature using each of the at least three detection channels; and
(c) determining the presence of target nucleic acids of the at least nine HPV types by the signals measured at the first detection temperature, the second detection temperature, and the third detection temperature using each of the at least three detection channels.

The present invention will be described in detail for each step:

Step (a): Incubation of Sample with Oligonucleotide Sets

First, in one reaction vessel, the sample is incubated with at least nine oligonucleotide sets to amplify or detect target nucleic acids of at least nine HPV types.

As used herein, the term “sample” refers to a cell, tissue or fluid from a biological source, or any other medium that can be beneficially evaluated in accordance with the invention. Specifically, the sample refers to a sample suspected to contain any one of the at least nine HPV types. The sample includes viruses, bacteria, tissues, cells, blood, scrum, plasma, lymph, milk, urine, feces, eye fluid, saliva, semen, brain extract, spinal fluid, appendix, spleen and tonsil tissue extract, amniotic fluid, ascites, and nonbiological samples (e.g., food and water). In addition, the sample contains naturally occurring nucleic acid molecules isolated from biological sources and synthesized nucleic acid molecules. In certain embodiments, the sample is a cervical swab sample or a liquid-based cytology sample. The term sample encompasses one harvested from a subject or one that has been subjected to a further process such as extraction, etc.

The method of the present disclosure is intended to determine whether any of target nucleic acids of at least nine HPV types are present in the sample.

As used herein, the term “target nucleic acids”, “target nucleic acid sequences” or “target sequences” refers to nucleic acids or their sequences to be detected or quantified. The target nucleic acid sequences include not only the newly generated sequences in the reaction, but also the sequences initially present in the nucleic acid sample.

The target nucleic acids herein are those derived from at least nine HPV types. The target nucleic acids of at least nine HPV types are double-stranded.

In this context, the target nucleic acids of at least nine HPV types refer to those from 9, 10, 11, 12, 13, 14 or 15 HPV types.

In an embodiment, the target nucleic acids include an internal control. The internal control can be used instead of one of the target nucleic acids of the HPV types. For example, when there are 9 target nucleic acids, the target nucleic acids may consist of target nucleic acids of 8 HPV types and an internal control; when there are 12 target nucleic acids, the target nucleic acids may consist of target nucleic acids of 11 HPV types and an internal control; when there are 15 target nucleic acids, the target nucleic acids may consist of target nucleic acids of 14 HPV types and an internal control.

In an embodiment, the target nucleic acids are from at least nine HPV types selected from the group consisting of high-risk HPV types and low-risk HPV types.

In certain embodiments, the target nucleic acids are from at least nine HPV types selected from HPV 6, 11, 16, 18, 26, 31, 33, 35, 39, 40, 42, 43, 44, 45, 51, 52, 53, 54, 56, 58, 59, 61, 66, 68, 69, 70, 73, and 82.

In certain embodiments, the target nucleic acids are from at least nine HPV types selected from the group consisting of HPV 16, 18, 31, 33, 35, 39, 45, 51, 52, 56, 58, 59, 66, 68, and an internal control.

In certain embodiments, the target nucleic acids consist of those from HPV 16, 18, 31, 33, 35, 39, 45, 51, 52, 56, 58, 59, 66, 68, and an internal control.

In certain embodiments, the target nucleic acids are from at least nine HPV types selected from the group consisting of HPV 6, 11, 26, 40, 42, 43, 44, 53, 54, 61, 69, 70, 73, 82, and an internal control.

In one embodiment, the target nucleic acids consist of those from HPV 6, 11, 26, 40, 42, 43, 44, 53, 54, 61, 69, 70, 73, 82, and an internal control.

The target nucleic acids can be subjected to known methods for separating double strands into single strands or partial single strands. Methods known to separate strands includes, but not limited to, heating, alkali, formamide, urea and glyoxal treatment, enzymatic methods (e.g., helicase action), and binding proteins. For instance, strand separation can be achieved by heating at temperature ranging from 80° C. to 105° C. General methods for accomplishing this treatment are provided by Joseph Sambrook, et al., Molecular Cloning, A Laboratory Manual, Cold Spring Harbor Laboratory Press, Cold Spring Harbor, N.Y. (2001).

According to the present disclosure, at least nine oligonucleotide sets are used to detect target nucleic acids of at least nine HPV types.

At least nine oligonucleotide sets herein refer to a collection of oligonucleotide sets specific for each of the target nucleic acids of at least nine HPV types. That is, one oligonucleotide set is specific for a target nucleic acid of one HPV type. The expression “one oligonucleotide set is specific for a target nucleic acid of one HPV type” means that the oligonucleotide set contributes to the detection of a target nucleic acid of one HPV type but does not contribute to the detection of a target nucleic acid of another HPV type. In other words, the expression “one oligonucleotide set is specific for a target nucleic acid of one HPV type” means that the oligonucleotide set interacts with a target nucleic acid of one HPV type but does not interact with a target nucleic acid of another HPV type. The above expression is not intended to mean that all oligonucleotides in the oligonucleotide set necessarily hybridize to the target nucleic acid.

Herein, a first oligonucleotide set is specific for a first target nucleic acid derived from one HPV type, a second oligonucleotide set is specific for a second target nucleic acid derived from another HPV type, and an n^{th} oligonucleotide set is specific for an n^{th} target nucleic acid derived from another HPV type.

The at least nine oligonucleotide sets are used together in one reaction, i.e., the at least nine oligonucleotide sets coexist in one reaction solution.

As used herein, the term “oligonucleotide set” refers to a plurality of oligonucleotides used in association with one target nucleic acid. Herein, one oligonucleotide set refers to a plurality of oligonucleotides used to amplify or detect one target.

Each of the at least nine oligonucleotide sets used in the present invention comprises:
(i) an amplifying oligonucleotide, which serves to amplify a target nucleic acid of an HPV type of interest; and
(ii) a signaling oligonucleotide having a fluorescent label linked thereto, which serves to generate a signal in the presence of a target nucleic acid of an HPV type of interest,
wherein the amplifying oligonucleotide and the signaling oligonucleotide are identical to or different from each other.

As an example, each of the at least nine oligonucleotide sets comprises an amplifying oligonucleotide or signaling oligonucleotide (e.g., a probe or primer) that is specifically hybridized to a target nucleic acid; if a probe or primer hybridized to a target nucleic acid is cleaved to release a fragment, it comprises a capture oligonucleotide specifically hybridized with the fragment; if the fragment hybridized with the capture oligonucleotide extends to form an extended strand, it comprises an oligonucleotide specifically hybridized with the extended strand, an oligonucleotide that is specifically hybridized with the capture oligonucleotide, or a combination thereof.

As used herein, the term “amplifying oligonucleotide” is used to collectively refer to an oligonucleotide which serves to amplify a target nucleic acid of the HPV type of interest.

In one embodiment, the amplifying oligonucleotide is a primer known in the art. The term “primer” as used herein refers to an oligonucleotide, which is capable of acting as a point of initiation of synthesis when placed under conditions in which synthesis of primer extension product which is complementary to a nucleic acid strand (template) is induced, i.e., in the presence of nucleotides and an agent for polymerization, such as DNA polymerase, and at a suitable temperature and pH. The primer must be sufficiently long to prime the synthesis of extension products in the presence of the agent for polymerization. The exact length of the primers will depend on many factors, including temperature, application, and source of primer.

The primer may include a forward primer (also referred to as an upstream primer or an upstream oligonucleotide), a reverse primer (also referred to as a downstream primer or a downstream oligonucleotide), or both.

The amplifying oligonucleotide may be an oligonucleotide having a structure known in the art and may be synthesized in a manner known in the art.

As used herein, the term “signaling oligonucleotide” is an oligonucleotide involved in the generation of the signal being detected. According to one embodiment, the signaling oligonucleotide comprises an oligonucleotide involved in actual signal generation. For example, hybridization or non-hybridization of signaling oligonucleotides with other oligonucleotides (e.g., oligonucleotides comprising nucleotide sequences complementary to target nucleic acids or signaling oligonucleotides) determines signal generation.

In one embodiment, the signaling oligonucleotide is a ‘probe’ known in the art. As used herein, the term “probe” refers to a single-stranded nucleic acid molecule comprising portion or portions substantially complementary to a target nucleic acid. According to one embodiment, the 3′-end of the probe is “blocked” to prevent its extension. The blocking may be achieved in accordance with conventional methods. For instance, the blocking may be performed by adding to the 3′-hydroxyl group of the last nucleotide a chemical moiety such as biotin, labels, a phosphate group, alkyl group, non-nucleotide linker, phosphorothioate or alkane-diol. Alternatively, the blocking may be carried out by removing the 3′-hydroxyl group of the last nucleotide or using a nucleotide with no 3′-hydroxyl group such as dideoxynucleotide.

Herein, the signaling oligonucleotide has at least one fluorescent label linked thereto, wherein the at least one fluorescent label in the signaling oligonucleotide generates a fluorescent signal.

In one embodiment, the signaling oligonucleotide consists of at least one oligonucleotide. According to an embodiment, when the signaling oligonucleotide consists of a plurality of oligonucleotides, the signaling oligonucleotide may have a label in various ways. For example, one of the plurality of oligonucleotides may have at least one label, all of the plurality of oligonucleotides may have at least one label, or one portion of the oligonucleotide may have at least one label and the other portion may not have a label.

According to the invention, the amplifying oligonucleotides and the signaling oligonucleotides included in each of the oligonucleotide sets are identical to or different from each other.

The expression “the amplifying oligonucleotide and the signaling oligonucleotide are identical to each other” means that one oligonucleotide serves not only as an amplifying oligonucleotide that amplifies a target nucleic acid, but also as a signaling oligonucleotide that generates a signal in the presence of the target nucleic acid. As an example, a labeled primer can hybridize with a target nucleic acid and then be extended to generate a signal.

The expression “the amplifying oligonucleotide and the signaling oligonucleotide are different from each other” means that two different oligonucleotides are used.

According to the present invention, the signaling oligonucleotide comprises a fluorescent label. The fluorescent label is linked to the signaling oligonucleotide.

In one embodiment, the at least nine oligonucleotide sets comprise oligonucleotide sets specific for HPV types selected from the group consisting of HPV 16, 18, 31, 33, 35, 39, 45, 51, 52, 56, 58, 59, 66, and 68.

In another embodiment, the at least nine oligonucleotide sets comprise oligonucleotide sets specific for HPV types selected from the group consisting of HPV 6, 11, 26, 40, 42, 43, 44, 53, 54, 61, 69, 70, 73, and 82.

In one embodiment, the at least nine oligonucleotide sets are fifteen (15) oligonucleotide sets.

In certain embodiments, the fifteen oligonucleotide sets consist of oligonucleotide sets specific for HPV 16, 18, 31, 33, 35, 39, 45, 51, 52, 56, 58, 59, 66, 68, and an internal control.

In certain embodiments, the fifteen oligonucleotide sets consist of oligonucleotide sets specific for HPV 6, 11, 26, 40, 42, 43, 44, 53, 54, 61, 69, 70, 73, 82, and an internal control.

According to the present disclosure, the oligonucleotide set comprising a signaling oligonucleotide may generate signals in a variety of ways well known in the art.

### (i) Generation of Signals by Formation or Dissociation of a Duplex

In one embodiment, at least one of the at least nine oligonucleotide sets generates a signal by formation or dissociation of a duplex from a signaling oligonucleotide, which occurs dependently on the presence of a target nucleic acid. In certain embodiments, all of the at least nine oligonucleotide sets generate signals by formation or dissociation of a duplex from a signaling oligonucleotide, which occurs dependently on the presence of the target nucleic acid.

According to one embodiment, the duplex comprises a double-stranded target nucleic acid.

The expression “generates a signal by formation or dissociation of a duplex from a signaling oligonucleotide, which occurs dependently on the presence of a target nucleic acid” as used herein in connection with an oligonucleotide set means that in the presence of a target nucleic acid, a signal is generated dependent on the formation of a duplex between the signaling oligonucleotide and the target nucleic acid or any nucleic acid derived therefrom, or on the dissociation of the formed duplex. The expression includes the provision of a signal by a duplex formed dependent on the presence of a target nucleic acid (e.g., a duplex between a nucleic acid and a signaling oligonucleotide having a label). In addition, the expression includes the provision of a signal by inhibition of the formation of a duplex (e.g., a duplex between a nucleic acid and a signaling oligonucleotide having a label), wherein the inhibition occurs by the formation of another duplex.

In particular, the signal is generated by the formation of a duplex between a target nucleic acid and a signaling oligonucleotide specifically hybridized with the target nucleic acid.

The signal by the formation of a duplex between a target nucleic acid and a signaling oligonucleotide may be generated by various methods, including Scorpion method (Whitcombe et al, Nature Biotechnology 17:804-807 (1999)), Sunrise (or Amplifluor) method (Nazarenko et al, Nucleic Acids Research, 25 (12): 2516-2521 (1997), and U.S. Pat. No. 6,117,635), Lux method (U.S. Pat. No. 7,537,886), Plexor method (Sherrill C B, et al., Journal of the American Chemical Society, 126:4550-45569 (2004)), Molecular Beacon method (Tyagi et al, Nature Biotechnology v. 14 Mar. 1996), HyBeacon method (French D J et al., Mol. Cell Probes, 15 (6): 363-374 (2001)), adjacent hybridization probe method (Bernard, P. S. et al., Anal. Biochem., 273:221 (1999)) and LNA method (U.S. Pat. No. 6,977,295).

According to one embodiment, at least one of the at least nine oligonucleotide sets generates a signal by cleavage of a mediation oligonucleotide hybridized to a target nucleic acid and subsequent formation or dissociation of a duplex between the cleavage product and a signaling oligonucleotide, which occurs dependently on the presence of the target nucleic acid. In certain embodiments, all of the at least nine oligonucleotide sets generate a signal by cleavage of a mediation oligonucleotide hybridized to a target nucleic acid and subsequent formation or dissociation of a duplex between the cleavage product and a signaling oligonucleotide, which occurs dependently on the presence of the target nucleic acid.

As used herein, the term “mediation oligonucleotide” is an oligonucleotide which mediates production of a duplex not containing a target nucleic acid.

According to an embodiment of the present invention, the cleavage of the mediation oligonucleotide per se does not generate any signal and a fragment formed by the cleavage is involved in successive reactions for signal generation following hybridization and cleavage of the mediation oligonucleotide.

According to an embodiment, the hybridization or cleavage of the mediation oligonucleotide per se does not generate a signal.

According to an embodiment of the present invention, the mediation oligonucleotide is hybridized with a target nucleic acid and cleaved to release a fragment, thereby mediating the production of a duplex. Particularly, the fragment mediates a production of a duplex by an extension of the fragment on a capture oligonucleotide.

According to an embodiment of the present invention, the mediation oligonucleotide comprises (i) a 3′-targeting portion comprising a hybridizing nucleotide sequence complementary to the target nucleic acid and (ii) a 5′-tagging portion comprising a nucleotide sequence non-complementary to the target nucleic acid.

According to an embodiment of the present invention, the cleavage of a mediation oligonucleotide releases a fragment, and the fragment is specifically hybridized with a capture oligonucleotide and extended on the capture oligonucleotide.

According to an embodiment of the present invention, a mediation oligonucleotide hybridized with target nucleic acids is cleaved to release a fragment, the fragment is specifically hybridized with a capture oligonucleotide and the fragment is extended to form an extended strand, resulting in formation of an extended duplex between the extended stand and the capture oligonucleotide, which provides a signal indicating the presence of the target nucleic acid. The capture oligonucleotide corresponds to the signaling oligonucleotide herein.

According to an embodiment of the present invention, where a third oligonucleotide comprising a hybridizing nucleotide sequence complementary to the extended strand is used, the hybridization of the third oligonucleotide with the extended strand forms another type of a duplex providing a signal indicating the presence of the target nucleic acid. The third oligonucleotide corresponds to the signaling oligonucleotide herein.

According to an embodiment of the present invention, where a third oligonucleotide comprising a hybridizing nucleotide sequence complementary to the capture oligonucleotide is used, the formation of a duplex between the third oligonucleotide and the capture oligonucleotide is inhibited by the formation of the duplex between the extended strand and the capturing oligonucleotide, thereby providing a signal indicating the presence of the target nucleic acid.

According to an embodiment of the present invention, the capture oligonucleotide, the third oligonucleotide, or a combination thereof can work as the signaling oligonucleotide.

The signal by the duplex formed in a dependent manner on cleavage of the mediation oligonucleotide may be generated by various methods, including PTOCE (PTO cleavage and extension) method (WO 2012/096523), PCE-SH (PTO Cleavage and Extension-Dependent Signaling Oligonucleotide Hybridization) method (WO 2013/115442) and PCE-NH (PTO Cleavage and Extension-Dependent Non-Hybridization) method (WO 2014/104818).

With referring to terms disclosed in the above references, the corresponding examples of the oligonucleotides are as follows: a mediation oligonucleotide is corresponding to a PTO (Probing and Tagging Oligonucleotide), a capture oligonucleotide to a CTO (Capturing and Templating Oligonucleotide), and a third oligonucleotide to SO (Signaling Oligonucleotide) or HO (Hybridization Oligonucleotide), respectively. SO, HO, CTO, or their combination may serve as a signaling oligonucleotide.

The signal by the duplex formed in a dependent manner on cleavage of the mediation oligonucleotide includes the signal provided by inhibition of the formation of another duplex by the duplex formed in a dependent manner on cleavage of the mediation oligonucleotide (e.g., PCE-NH).

For example, where the signal by the duplex formed in a dependent manner on cleavage of the mediation oligonucleotide is generated by PTOCE method, an upstream oligonucleotide, a PTO (Probing and Tagging Oligonucleotide) comprising a hybridizing nucleotide sequence complementary to the target nucleic acid, a CTO (Capturing and Templating Oligonucleotide), a suitable label and a template-dependent nucleic acid polymerase having 5′ nuclease activity are included in the reaction.

The PTO comprises (i) a 3′-targeting portion comprising a hybridizing nucleotide sequence complementary to the target nucleic acid and (ii) a 5′-tagging portion comprising a nucleotide sequence non-complementary to the target nucleic acid. The CTO comprises in a 3′ to 5′ direction (i) a capturing portion comprising a nucleotide sequence complementary to the 5′-tagging portion or a part of the 5′-tagging portion of the PTO and (ii) a templating portion comprising a nucleotide sequence noncomplementary to the 5′-tagging portion and the 3′-targeting portion of the PTO.

The particular example of the signal generation by PTOCE method comprises the steps of:
(a) hybridizing the target nucleic acid with the upstream oligonucleotide and the PTO; (b) contacting the resultant of the step (a) to an enzyme having 5′ nuclease activity under conditions for cleavage of the PTO; wherein the upstream oligonucleotide or its extended strand induces cleavage of the PTO by the enzyme having 5′ nuclease activity such that the cleavage releases a fragment comprising the 5′-tagging portion or a part of the 5′-tagging portion of the PTO; (c) hybridizing the fragment released from the PTO with the CTO; wherein the fragment released from the PTO is hybridized with the capturing portion of the CTO; and (d) performing an extension reaction using the resultant of the step (c) and a template-dependent nucleic acid polymerase; wherein the fragment hybridized with the capturing portion of the CTO is extended and an extended duplex is formed; wherein the extended duplex has a Tₘ value adjustable by (i) a sequence and/or length of the fragment, (ii) a sequence and/or length of the CTO or (iii) the sequence and/or length of the fragment and the sequence and/or length of the CTO; wherein the extended duplex provides a target signal by at least one label linked to the fragment and/or the CTO; and (e) detecting the extended duplex by measuring the target signal at a predetermined temperature that the extended duplex maintains its double-stranded form, whereby the presence of the extended duplex indicates the presence of the target nucleic acid. In this case, the method further comprises repeating all or some of the steps (a)-(c) with denaturation between repeating cycles.

In the phrase “denaturation between repeating cycles”, the term “denaturation” means to separate a double-stranded nucleic acid molecule to a single-stranded nucleic acid molecule.

In the step (a) of PTOCE method, a primer set for amplification of the target nucleic acid may be used instead of the upstream oligonucleotide. In this case, the method further comprises repeating all or some of the steps (a)-(c) with denaturation between repeating cycles.

The PTOCE method can be classified as a process in which the PTO fragment hybridized with the CTO is extended to form an extended strand and the extended strand is then detected. The PTOCE method is characterized in that the formation of the extended strand is detected by using the duplex between the extended strand and the CTO.

There is another approach to detect the formation of the extended strand. For example, the formation of the extended strand may be detected by using an oligonucleotide specifically hybridized with the extended strand (e.g., PCE-SH method). In this method, the signal may be provided from (i) a label linked to the oligonucleotide specifically hybridized with the extended strand, or (ii) a label linked to the oligonucleotide specifically hybridized with the extended strand and a label linked to the PTO fragment.

Alternatively, the detection of the formation of the extended strand is performed by another method in which inhibition of the hybridization between the CTO and an oligonucleotide being specifically hybridizable with the CTO is detected (e.g., PCE-NH method). Such inhibition is considered to be indicative of the presence of a target nucleic acid. The signal may be provided from (i) a label linked to the oligonucleotide being hybridizable with the CTO, (ii) a label linked to the CTO, or (iii) a label linked to the oligonucleotide being hybridizable with the CTO and a label linked to the CTO.

According to an embodiment, the oligonucleotide being specifically hybridizable with the CTO has an overlapping sequence with the PTO fragment.

According to an embodiment, the signaling oligonucleotide includes the oligonucleotide being specifically hybridizable with the extended strand (e.g., PCE-SH method) and oligonucleotide being specifically hybridizable with the CTO (e.g., PCE-NH method).

The PTOCE-based methods commonly involve the formation of the extended strand depending on the presence of a target nucleic acid. The term “PTOCE-based method” is used herein to intend to encompass various methods for providing signals, comprising the formation of an extended strand through cleavage and extension of PTO.

The example of signal generation by the PTOCE-based methods comprises the steps of: (a) hybridizing the target nucleic acid with the upstream oligonucleotide and the PTO; (b) contacting the resultant of the step (a) to an enzyme having 5′ nuclease activity under conditions for cleavage of the PTO; wherein the upstream oligonucleotide or its extended strand induces cleavage of the PTO by the enzyme having 5′ nuclease activity such that the cleavage releases a fragment comprising the 5′-tagging portion or a part of the 5′-tagging portion of the PTO; (c) hybridizing the fragment released from the PTO with the CTO; wherein the fragment released from the PTO is hybridized with the capturing portion of the CTO; (d) performing an extension reaction using the resultant of the step (c) and a template-dependent nucleic acid polymerase; wherein the fragment hybridized with the capturing portion of the CTO is extended to form an extended strand; and (e) detecting the formation of the extended strand by detecting signal generated dependent on the presence of the extended strand. In the step (a), a primer set for amplification of the target nucleic acid may be used instead of the upstream oligonucleotide. In this case, the method further comprises repeating all or some of the steps (a)-(e) with denaturation between repeating cycles.

According to an embodiment, the signal generated by the formation of a duplex includes signals induced by hybridization of the duplex (e.g., hybridization of the duplex per se, or hybridization of a third oligonucleotide) or by inhibition of hybridization of a third oligonucleotide due to the formation of a duplex.

According to one embodiment, at least one of the at least nine oligonucleotide sets generates a signal by cleavage of the signaling oligonucleotide hybridized to the target nucleic acid and then dissociation of a duplex, which occurs dependently on the presence of the target nucleic acid.

According to a particular embodiment, all of the at least nine oligonucleotide sets generates a signal by cleavage of the signaling oligonucleotide hybridized to the target nucleic acid and then dissociation of a duplex, which occurs dependently on the presence of the target nucleic acid.

By way of example, the signaling oligonucleotide may include a double-stranded tagging portion comprising a non-complementary nucleotide sequence to the target nucleic acid, a single-stranded targeting portion comprising a hybridizing nucleotide sequence complementary to the target nucleic acid, and a label.

In one embodiment, the label may be interactive triple labels comprising one reporter molecule and two quencher molecules.

According to one embodiment, in the presence of a target nucleic acid, either or both quencher molecules of the interactive triple labels is in close proximity to the reporter molecule to quench the signal from the reporter molecule at a first detection temperature, and both quencher molecules are separated from the reporter molecule to unquench the signal from the reporter molecule at a second temperature.

According to one embodiment, one quencher molecule of the interactive triple labels may be linked to a targeting portion of the signaling oligonucleotide, and another quencher molecule and a reporter molecule of the interactive triple labels may be linked to positions where they can be released together when the tagging portion of the signaling oligonucleotide is cleaved to be released. In particular, the reporter molecule of the interactive triple labels may be linked to a position where it can reversibly interact with the quencher molecule linked to the targeting portion.

According to a specific embodiment, one reporter molecule and one quencher molecule of the interactive triple labels are linked to a double-stranded tagging portion of the signaling oligonucleotide, and the remaining quencher molecule of the interactive triple labels may be linked to the targeting portion of the signaling oligonucleotide. In particular, one reporter molecule and one quencher molecule may be linked to positions of the tagging portion where they can reversibly interact.

For example, when a target nucleic acid is present, one reporter molecule and one quencher molecule of the interactive triple labels are separated from the remaining quencher molecule by cleavage of the double-stranded tagging portion of the signaling oligonucleotide. The cleaved double-stranded tagging portion maintains a double-stranded form or dissociates into a single-stranded form depending on the temperature. Specifically, (i) the cleaved double-stranded tagging portion maintains a double-stranded form such that the reporter molecule and the quencher molecule linked to the tagging portion are in close proximity to each other at a temperature lower than its Tₘ, allowing the quencher molecule to quench a signal from the reporter molecule, and (ii) the cleaved double-stranded tagging portion is dissociated into a single-stranded form such that the reporter molecule and the quencher molecule are separated from each other at a temperature higher than its Tₘ, allowing the quencher molecule to unquench a signal from the reporter molecule.

In one embodiment, cleavage of the double-stranded tagging portion of the signaling oligonucleotide may be performed using a polymerase having nuclease activity or may be performed using a separate nuclease but is not limited thereto. The nuclease may be a naturally occurring, unmodified or modified nuclease.

One example of signal generation by dissociation of duplexes after cleavage of signaling oligonucleotides hybridized with the target nucleic acid, which occurs dependently on the presence of the target nucleic acid are found in U.S. Pat. Nos. 11,034,997, 11,028,433 and 10,590,469, and U.S. Appln. Pub. No. 2020-0048682.

(ii) Signal Generation by Cleavage after Hybridization

According to one embodiment, at least one of the at least nine oligonucleotide sets generates a signal by hybridization of the signaling oligonucleotide with a target nucleic acid and then cleavage of the signaling oligonucleotide, which occurs dependently on the presence of the target nucleic acid.

The signal by hybridization of the signaling oligonucleotide with a target nucleic acid and then cleavage of the signaling oligonucleotide may be generated by various methods, including TaqMan probe method (U.S. Pat. Nos. 5,210,015 and 5,538,848).

Where the signal is generated by TaqMan probe method, a primer set for amplification of a target nucleic acid, a TaqMan probe having a suitable label (e.g., interactive dual labels) and a nucleic acid polymerase having 5′-nuclease activity are included in a reaction. The TaqMan probe hybridized with a target nucleic acid is cleaved during target amplification and generates a signal indicating the presence of the target nucleic acid.

The particular example of generating a signal by TaqMan probe method comprises the steps of: (a) hybridizing the primer set and the TaqMan probe having a suitable label (e.g., interactive dual labels) with the target nucleic acid; (b) amplifying the target nucleic acid by using the resultant of the step (a) and the nucleic acid polymerase having 5′-nuclease activity, wherein the TaqMan probe is cleaved to release the label; and (c) detecting a signal generation from the released label.

Particularly, the signal is generated by cleavage of the signaling oligonucleotide in a dependent manner on cleavage of a mediation oligonucleotide specifically hybridized with the target nucleic acid.

According to an embodiment of the present invention, where a mediation oligonucleotide hybridized with target nucleic acid is cleaved to release a fragment, the fragment is specifically hybridized with a signaling oligonucleotide and the fragment induces the cleavage of the signaling oligonucleotide.

According to an embodiment of the present invention, where a mediation oligonucleotide hybridized with target nucleic acid is cleaved to release a fragment, the fragment is extended to cleave a signaling oligonucleotide comprising a hybridizing nucleotide sequence complementary to the capture oligonucleotide.

The signal by cleavage of the signaling oligonucleotide in a dependent manner on cleavage of the mediation oligonucleotide may be generated by various methods, including Invader assay (U.S. Pat. No. 5,691,142), PCEC (PTO Cleavage and Extension-Dependent Cleavage) method (WO 2012/134195) and a method described in U.S. Pat. No. 7,309,573. In particular, the method described in U.S. Pat. No. 7,309,573 may be considered as one of PTOCE-based methods using signal generation by cleavage, and in the method, the formation of the extended strand may be detected by detecting cleavage of an oligonucleotide specifically hybridized with the CTO by the formation of the extended strand. Invader assay forms a fragment by cleavage of a mediation oligonucleotide and induces successive cleavage reactions with no extension of the fragment.

According to an embodiment of the present invention, where the signal is generated in a dependent manner on cleavage of a signaling oligonucleotide, the cleavage of the signaling oligonucleotide induces signal changes or releases a labeled fragment to be detected.

Where an oligonucleotide set generates a signal simultaneously by cleavage of a signaling oligonucleotide and by the formation of a duplex, the oligonucleotide set may be considered to provide a signal by cleavage, so long as it is used to generate signal by cleavage.

At least nine oligonucleotide sets used herein may adopt various signal generation methods described above.

By way of example, all of the at least nine oligonucleotide sets may generate a signal by formation or dissociation of a duplex from the signaling oligonucleotide, which occurs dependently on the presence of a target nucleic acid. As another example, some of the at least nine oligonucleotide sets may generate a signal by formation or dissociation of a duplex from the signaling oligonucleotide, which occurs dependently on the presence of a target nucleic acid, and the remainder may generate a signal by hybridization of a signaling oligonucleotide with a target nucleic acid and then cleavage of the signaling oligonucleotide, which occurs dependently on the presence of the target nucleic acid.

According to the present method, for three oligonucleotide sets comprising any one of the at least three different type of the fluorescent labels, one oligonucleotide set is designed to generate a signal at a first detection temperature in the presence of a target nucleic acid of a corresponding HPV type, another oligonucleotide set is designed to generate a signal at the first detection temperature and a second detection temperature in the presence of a target nucleic acid of another corresponding HPV type, and the other oligonucleotide set is designed to generate a signal at the first detection temperature, the second detection temperature and a third detection temperature in the presence of a target nucleic acid of the other corresponding HPV type;

Those skilled in the art can choose a signal generation method for the oligonucleotide sets to satisfy these requirements.

For example, if an oligonucleotide set that generates a signal by hybridization of a signaling oligonucleotide with a target nucleic acid and then cleavage of the signaling oligonucleotide, which occurs dependently on the presence of a target nucleic acid, generates a signal at a first detection temperature, a second detection temperature and a third detection temperature, the oligonucleotide set is used for a target nucleic acid having the third detection temperature and not used for a target nucleic acid having the first detection temperature or a target nucleic acid having the second detection temperature.

As another example, if an oligonucleotide set that generates a signal by dissociation of the duplex after cleavage of the signaling oligonucleotide hybridized with the target nucleic acid, which occurs dependently on the presence of the target nucleic acid, generates a signal at a first detection temperature, the oligonucleotide set is used for a target nucleic acid having the first detection temperature; if an oligonucleotide set generates a signal at a first detection temperature and a second detection temperature, the oligonucleotide set is used for a target nucleic acid having the second detection temperature; if an oligonucleotide set generates a signal at a first detection temperature, a second detection temperature and a third detection temperature, the oligonucleotide set is used for a target nucleic acid having the third detection temperature.

According to one embodiment, for three oligonucleotide sets comprising the same type of fluorescent label, there is one oligonucleotide set that generates a signal by hybridization of the signaling oligonucleotide with the target nucleic acid and then cleavage of the signaling oligonucleotide, which occurs dependently on the presence of the target nucleic acid.

According to one embodiment, for three oligonucleotide sets comprising the same type of fluorescent label, an oligonucleotide set for the target nucleic acid having the third detection temperature is an oligonucleotide set that generates a signal by cleavage of the signaling oligonucleotide, and an oligonucleotide set for the target nucleic acid having the first detection temperature and an oligonucleotide set for the target nucleic acid having the second detection temperature are oligonucleotide sets that generate a signal by formation of the duplex.

According to one embodiment, for three oligonucleotide sets comprising the same type of fluorescent label, an oligonucleotide set for the target nucleic acid having the third detection temperature is an oligonucleotide set that generates a signal by cleavage of the signaling oligonucleotide, and an oligonucleotide set for the target nucleic acid having the first detection temperature and an oligonucleotide set for the target nucleic acid having the second detection temperature are oligonucleotide sets that generate a signal by formation of duplexes in a manner dependent on cleavage of a mediated oligonucleotide specifically hybridized to the target nucleic acid.

According to the method of the present invention, the fluorescent labels included in the at least nine oligonucleotide sets are of at least three different types, which are distinguishable by at least three different detection channels, and three of the at least nine oligonucleotide sets comprise any one of the at least three different types of the fluorescent labels.

As used herein, a single or one type of fluorescent label refers to fluorescent labels having identical or substantially identical signal properties (e.g., optical properties, emission wavelength, and electrical signal). For example, FAM and CAL Fluor 610 provide different types of signals. Such single or one type of fluorescent label does not depend on the chemical structure of the fluorescent label. Thus, two fluorescent labels having different chemical structures can be also considered to be of one type as long as they generate signals that are not distinguished using a detection channel.

According to the present invention, signals generated from three oligonucleotide sets comprising the same type of fluorescent label are not distinguished by one detection channel.

As used herein, the term “detection channel” refers to a means for detecting a signal from a single type of fluorescent label. Thermocyclers available in the art, such as ABI (Applied Biosystems), QuantStudio (Applied Biosystems), CFX96 (Bio-Rad Laboratories), cobas Z 480 (Roche), LightCycler (Roche), etc., include several channels (e.g., photodiodes) for detecting signals from several different types of fluorescent labels, and said channel corresponds to the detection channel herein.

Fluorescent labels useful in the present invention include various fluorescent labels known in the art. For example, fluorescent labels useful in the present invention include a single fluorescent label, interactive dual fluorescent labels and interactive triple labels.

In one embodiment, the single label provides different signal intensities depending on its presence on a double strand or single strand. The preferable types and binding sites of single fluorescent labels used in this invention are disclosed U.S. Pat. Nos. 7,537,886 and 7,348,141, the teachings of which are incorporated herein by reference in their entirety. For example, the single fluorescent label includes JOE, FAM, TAMRA, ROX and fluorescein-based label. The single label may be linked to oligonucleotides by various methods. For instance, the label is linked to probes through a spacer containing carbon atoms (e.g., 3-carbon spacer, 6-carbon spacer or 12-carbon spacer).

As a representative of the interactive label system, the FRET (fluorescence resonance energy transfer) label system includes a fluorescent reporter molecule (donor molecule) and a quencher molecule (acceptor molecule). In FRET, the energy donor is fluorescent, but the energy acceptor may be fluorescent or non-fluorescent. In another form of interactive label systems, the energy donor is non-fluorescent, e.g., a chromophore, and the energy acceptor is fluorescent. In yet another form of interactive label systems, the energy donor is luminescent, e.g., bioluminescent, chemiluminescent, electrochemiluminescent, and the acceptor is fluorescent. The interactive label system includes a dual label based on “on contact-mediated quenching” (Salvatore et al., Nucleic Acids Research, 2002 (30) no. 21 e122 and Johansson et al., J. AM. CHEM. SOC 2002 (124) pp 6950-6956). The interactive label system includes any label system in which signal change is induced by interaction between at least two molecules (e.g., dye).

The reporter molecule and the quencher molecule useful in the present invention may include any molecules known in the art. Examples of those are: Cy2™ (506), YOPRO™-1 (509), YOYO™-1 (509), Calcein (517), FITC (518), FluorX™ (519), Alexa™ (520), Rhodamine 110 (520), Oregon Green™ 500 (522), Oregon Green™ (524), RiboGreen™ (525), Rhodamine Green™ (527), Rhodamine 123 (529), Magnesium Green™ (531), Calcium Green™ (533), TO-PRO™-1 (533), TOTO1 (533), JOE (548), BODIPY530/550 (550), Dil (565), BODIPY TMR (568), BODIPY558/568 (568), BODIPY564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAMRA (582), Rhodamine Red™ (590), Cy3.5™ (596), ROX (608), Calcium Crimson™ (615), Alexa™ 594 (615), Texas Red (615), Nile Red (628), YO-PRO™-3 (631), YOYO™-3 (631), R-phycocyanin (642), C-Phycocyanin (648), TO-PRO™-3 (660), TOTO3 (660), DID DilC (5) (665), Cy5™ (670), Thiadicarbocyanine (671), Cy5.5 (694), HEX (556), TET (536), Biosearch Blue (447), CAL Fluor Gold 540 (544), CAL Fluor Orange 560 (559), CAL Fluor Red 590 (591), CAL Fluor Red 610 (610), CAL Fluor Red 635 (637), FAM (520), Fluorescein (520), Fluorescein-C3 (520), Pulsar 650 (566), Quasar 570 (667), Quasar 670 (705) and Quasar 705 (610). The numeric in parenthesis is a maximum emission wavelength in nanometer.

Preferably, the reporter molecule and the quencher molecule include JOE, FAM, TAMRA, ROX and fluorescein-based label.

Suitable fluorescence molecule and suitable pairs of reporter-quencher are disclosed in a variety of publications as follows: Pesce et al., editors, Fluorescence Spectroscopy (Marcel Dekker, New York, 1971); White et al., Fluorescence Analysis: A Practical Approach (Marcel Dekker, New York, 1970); Berlman, Handbook of Fluorescence Spectra of Aromatic Molecules, 2nd Edition (Academic Press, New York, 1971); Griffiths, Color AND Constitution of Organic Molecules (Academic Press, New York, 1976); Bishop, editor, Indicators (Pergamon Press, Oxford, 1972); Haugland, Handbook of Fluorescent Probes and Research Chemicals (Molecular Probes, Eugene, 1992); Pringsheim, Fluorescence and Phosphorescence (Interscience Publishers, New York, 1949); Haugland, R. P., Handbook of Fluorescent Probes and Research Chemicals, 6th Edition (Molecular Probes, Eugene, Oreg., 1996) U.S. Pat. Nos. 3,996,345 and 4,351,760.

It is noteworthy that a non-fluorescent quencher molecule (e.g., black quencher or dark quencher) capable of quenching a fluorescence of a wide range of wavelengths or a specific wavelength may be used in the present invention.

In the signaling system comprising the reporter and quencher molecules, the reporter encompasses a donor of FRET and the quencher encompasses the other partner (acceptor) of FRET. For example, a fluorescein dye is used as the reporter and a rhodamine dye as the quencher.

The interactive dual labels may be linked to one strand of a duplex. Where the strand containing the interactive dual labels leaves in a single stranded state, it forms a hairpin or random coil structure to induce quenching between the interactive dual labels. Where the strand forms a duplex, the quenching is relieved. Alternatively, where the interactive dual labels are linked to nucleotides adjacently positioned on the strand, the quenching between the interactive dual labels occurs. Where the strand forms a duplex and then is cleaved, the quenching becomes relieved.

A reporter molecule and a quencher molecule of the interactive dual labels may be each linked to each of two strands of the duplex. For example, a reporter molecule is linked to one strand of the duplex and a quencher molecule is linked to the other strand of the duplex. The formation of the duplex induces quenching and denaturation of the duplex induces unquenching. Alternatively, where one of the two stands is cleaved, the unquenching may be induced.

In one embodiment, the fluorescent labels included in the at least nine oligonucleotide sets are of at least three different types selected from the group consisting of FAM or an equivalent thereof, CAL Fluor Orange 560 or an equivalent thereof, CAL Fluor Red 610 or an equivalent thereof, Quasar 670 or an equivalent thereof, and Quasar 705 or an equivalent thereof.

In another embodiment, the fluorescent labels included in the at least nine oligonucleotide sets are FAM or an equivalent thereof, CAL Fluor Orange 560 or an equivalent thereof, CAL Fluor Red 610 or an equivalent thereof, Quasar 670 or an equivalent thereof, and Quasar 705 or an equivalent thereof. The five types of fluorescent labels can be used to detect up to 15 target nucleic acids, such as 14 HPV types and 1 internal control.

The term “equivalent thereof” in the context of fluorescent label refers to any fluorescent label that can be used in lieu of the fluorescent label as described above, including a fluorescent label that does not significantly affect the detection result when used in place of the fluorescent label as described above. As an example, another fluorescent label exhibiting signal properties (e.g., optical properties, emission wavelength, and electrical signal) identical to or substantially identical to the fluorescent label as described above may be included in the definition.

The equivalents of the fluorescent labels used herein are known in the art. For example, equivalents of FAM fluorescent label include Alexa Fluor 488, Cy2, Oregon Green, ATTO 465, etc., equivalents to CAL Fluor Orange 560 include VIC, HEX, Yakima Yellow, ATTO 532, etc., equivalents of CAL Fluor Red 610 include Pet, ROX, Cy 3.5, ATTO 565, LC Red 610, Texas Red, Alexa Fluor 594, etc., equivalents to Quasar 670 include Cy 5, ATTO647N, etc., and equivalents to Quasar 705 include Cy 5.5, ATTO 680, ATTO 700, etc.

According to an embodiment of this invention, step (a) is performed in a signal amplification process concomitantly with a nucleic acid amplification.

In the present invention, the signal generated by signal-generating means may be amplified simultaneously with target amplification. Alternatively, the signal may be amplified with no target amplification.

According to an embodiment of this invention, the signal generation is performed in a process involving signal amplification together with target amplification.

According to an embodiment of this invention, the target amplification is performed in accordance with PCR (polymerase chain reaction). PCR is widely employed for target amplification in the art, including cycles of denaturation of a target sequence, annealing (hybridization) between the target sequence and primers, and primer extension (Mullis et al. U.S. Pat. Nos. 4,683,195, 4,683,202 and 4,800,159; Saiki et al., (1985) Science 230, 1350-1354). The signal may be amplified by applying the signal generation methods described above (e.g., TaqMan method and PTOCE-based methods) to the PCR process.

According to an embodiment, the amplification of the target nucleic acid is performed by PCR (polymerase chain reaction), LCR (ligase chain reaction, see Wiedmann M, et al., “Ligase chain reaction (LCR)-overview and applications.” PCR Methods and Applications 1994 February; 3 (4): S51-64), GLCR (gap filling LCR, see WO 90/01069, EP 439182 and WO 93/00447), Q-beta (Q-beta replicase amplification, see Cahill P, et al., Clin Chem., 37 (9): 1482-5 (1991), U.S. Pat. No. 5,556,751), SDA (strand displacement amplification, see G T Walker et al., Nucleic Acids Res. (7): 16911696 (1992), EP 497272), NASBA (nucleic acid sequence-based amplification, see Compton, J. Nature 350 (6313): 912 (1991)), TMA (Transcription-Mediated Amplification, see Hofmann W P et al., J Clin Virol. 32 (4): 289-93 (2005); U.S. Pat. No. 5,888,779).) or RCA (Rolling Circle Amplification, see Hutchison C. A. et al., Proc. Natl Acad. Sci. USA. 102:1733217336 (2005)), LAMP (loop-mediated isothermal amplification, see Y. Mori, H. Kanda and T. Notomi, J. Infect. Chemother., 2013, 19, 404-411), RPA (recombinase polymerase amplification, see J. Li, J. Macdonald and F. von Stetten, Analyst, 2018, 144, 31-67), or HAD (helicase dependent amplification, see M. Vincent, Y. Xu and H. Kong, EMBO Rep., 2004, 5, 795-800).

The amplification methods described above may amplify target nucleic acids through repeating a series of reactions with or without changing temperatures. The unit of amplification comprising the repetition of a series of reactions is expressed as a “cycle”. The unit of cycles may be expressed as the number of the repetition or time being dependent on amplification methods.

For example, the detection of signals may be performed at each cycle of amplification, selected several cycles or end-point of reactions.

According to an embodiment, where signals are detected at at least two cycles, the detection of signal in an individual cycle may be performed at all detection temperatures or some selected detection temperatures.

According to an embodiment of this invention, step (a) is performed under the conditions allowing target amplification well as signal generation by the oligonucleotide sets.

According to an embodiment of this invention, step (a) is performed in a signal amplification process without a nucleic acid amplification.

Where the signal is generated by methods including cleavage of an oligonucleotide, the signal may be amplified with no target amplification. For example, step (a) may be performed with amplification of signals but with no amplification of target sequences in accordance with CPT method (Duck P, et al., Biotechniques, 9:142-148 (1990)), Invader assay (U.S. Pat. Nos. 6,358,691 and 6,194,149), PTOCE-based methods (e.g., PCE-SH method, PCE-NH method and PCEC method) or CER method (WO 2011/037306).

In a particular embodiment, the amplification may be performed by real-time PCR in which Ct values are provided for each of target nucleic acids of the at least nine HPV types.

The amplification of the target nucleic acid is accomplished by an amplifying oligonucleotide and a nucleic acid polymerase.

According to an embodiment of the present invention, a nucleic acid polymerase having nuclease activity (e.g., 5′ nuclease activity or 3′ nuclease activity) may be used. According to an embodiment of the present invention, a nucleic acid polymerase having no nuclease activity may be used.

The nucleic acid polymerase useful in the present invention is a thermostable DNA polymerase obtained from a variety of bacterial species, including, without limitation, Thermus aquaticus (Taq), Thermus thermophilus (Tth), Thermus filiformis, Thermus flavus, Thermococcus literalis, Thermus antranikianii, Thermus caldophilus, Thermus chliarophilus, Thermus igniterrae, Thermus lacteus, Thermus oshimai, Thermus ruber, Thermus rubens, Thermus scotoductus, Thermus silvanus, Thermus species Z05, Thermus species sps 17, Thermotoga maritima, Thermotoga neapolitana, Thermosipho africanus, Thermococcus litoralis, Thermococcus barossi, Thermococcus gorgonarius, Pyrococcus woesei, Pyrococcus horikoshii, Pyrococcus abyssi, Pyrodictium occultum, Aquifex pyrophilus and Aquifex aeolieus. Particularly, the thermostable DNA polymerase is Taq polymerase.

According to an embodiment of the present invention, the amplification of the target nucleic acid is accomplished by an asymmetric PCR.

The ratio of amplifying oligonucleotides may be selected in consideration of cleavage or hybridization of downstream oligonucleotides.

During or after the incubation (reaction) of the sample with the at least nine oligonucleotide sets to generate a signal, the generated signal is detected by using at least three different detection channels.

According to the present invention, three of the at least nine oligonucleotide sets comprise any one of the at least three different types of the fluorescent labels. In this case, for three oligonucleotide sets comprising any one of the at least three different types of the fluorescent labels, one oligonucleotide set is designed to generate a signal at a first detection temperature in the presence of a target nucleic acid of a corresponding HPV type, another oligonucleotide set is designed to generate a signal at the first detection temperature and a second detection temperature in the presence of a target nucleic acid of another corresponding HPV type, and the other oligonucleotide set is designed to generate a signal at the first detection temperature, the second detection temperature and a third detection temperature in the presence of a target nucleic acid of the other corresponding HPV type.

For example, when among nine oligonucleotide sets, three oligonucleotide sets include FAM fluorescent labels, three other oligonucleotide sets include CAL Fluor Orange 560 fluorescent labels, and the remaining three oligonucleotide sets include Quasar 670 fluorescent labels, for the three oligonucleotide sets including the FAM fluorescent labels, a first oligonucleotide set generates a signal at the first detection temperature but not at the second detection temperature and the third detection temperature in the presence of a first HPV type, a second oligonucleotide set generates a signal at the first detection temperature and the second detection temperature but not at the third detection temperature in the presence of a second HPV type, and a third oligonucleotide set generates a signal at the first detection temperature, the second detection temperature and the third detection temperature in the presence of a third HPV type. The same applies to the three other oligonucleotide sets including CAL Fluor Orange 560 fluorescent labels and the remaining three oligonucleotide sets including Quasar 670 fluorescent labels.

For the three oligonucleotide sets comprising the same type of fluorescent label, the target nucleic acid of the HPV type generating a signal at the first detection temperature has the first detection temperature determined by the corresponding oligonucleotide set, the target nucleic acid of the HPV type generating a signal at the first detection temperature and the second detection temperature has the second detection temperature determined by the corresponding oligonucleotide set, and the target nucleic acid of the HPV type generating a signal at the first detection temperature, the second detection temperature, and the third detection temperature has the third detection temperature determined by the corresponding oligonucleotide set.

As used herein, the expression “a target nucleic acid has a detection temperature determined by the corresponding oligonucleotide set” means that a target nucleic acid is detectable at a detection temperature pre-assigned to the target nucleic acid.

According to an embodiment of the present invention, one detection temperature determined by the corresponding oligonucleotide set is assigned to one target nucleic acid.

The first detection temperature is a temperature capable of generating a signal for an HPV type having the first detection temperature, a signal for an HPV type having the second detection temperature and a signal for an HPV type having the third detection temperature; the second detection temperature is a temperature capable of generating a signal for the HPV type having the second detection temperature and a signal for the HPV type having the third detection temperature; the third detection temperature is a temperature capable of generating a signal for the HPV type having the third detection temperature. In other words, signals for all the three HPV types are detected at the first detection temperature, signals for the two HPV types are detected at the second detection temperature, and a signal for one HPV type is detected at the third detection temperature.

One of features of the present invention is to determine differentially the presence of three HPV types by signals measured at three detection temperatures.

According to an embodiment, the three detection temperatures are predetermined based on a temperature range to allow signal generation by the oligonucleotide set.

The method of the present invention is based on the fact that there is a certain temperature range to allow signal generation in a dependent manner on the oligonucleotide set.

For example, when an oligonucleotide set generates a signal upon hybridization (or association) and do not generate a signal upon non-hybridization (or dissociation), a signal is generated at temperatures at which the formation of a duplex is allowed, but no signal is generated at temperatures at which the formation of a duplex fails. As such, there is a certain temperature range to allow signal generation (i.e., signal detection) and other temperature range not to allow signal generation. The temperature ranges are affected by the Tₘ value of the signaling oligonucleotide included in the oligonucleotide set.

Where the signal generation method using a released fragment with a label after cleavage is employed, the signal may be theoretically detected at any temperature (e.g., 30-99° C.).

A detection temperature is selected from the temperature range to allow signal generation by the oligonucleotide set.

The term “detection temperature range” is used herein to particularly describe the temperature range to allow signal generation (i.e., signal detection).

Where three oligonucleotide sets for three HPV types have different detection temperature ranges, the first detection temperature may be selected from a range in which a detection temperature range at which a signal is generated from the first oligonucleotide set, a detection temperature range at which a signal is generated from the second oligonucleotide set, and a detection temperature range at which a signal is generated from the third oligonucleotide set are overlapping; the second detection temperature may be selected from a range in which a detection temperature range at which a signal is generated from the second oligonucleotide set and a detection temperature range at which a signal is generated from the third oligonucleotide set are overlapping; and the third detection temperature may be selected from a detection temperature range at which a signal is generated only from the third oligonucleotide set.

According to one embodiment, the first detection temperature, the second detection temperature, and the third detection temperature may be predetermined. For example, the first detection temperature, the second detection temperature, and the third detection temperature are predetermined to be 60° C., 72° C. and 83° C., respectively, and then oligonucleotide sets suitable for the detection temperatures are constructed, followed by performing step (a).

According to an embodiment, oligonucleotide sets for the three HPV types are firstly constructed and then detection temperatures for the three target nucleic acids are allocated, followed by performing step (a).

According to an embodiment of this invention, when the oligonucleotide set generates a signal in a dependent manner on the formation of a duplex, the detection temperature is selected based on a Tₘ value of the duplex.

According to an embodiment of this invention, when the oligonucleotide set generates a signal in a dependent manner on the formation of a duplex, the detection temperature is controllable by adjusting a Tₘ value of the duplex.

For example, where the signal is generated by a signaling oligonucleotide specifically hybridized with the target nucleic acid (e.g., Lux probe, Molecular Beacon probe, HyBeacon probe and adjacent hybridization probe), the detection of the signal is successfully done at the predetermined temperature by adjusting the Tₘ value of the signaling oligonucleotide.

Where Scorpion primer is used, the detection of the signal is successfully done at the predetermined temperature by adjusting the Tₘ value of a portion to be hybridized with extended strand.

Where the signal is generated by the duplex formed dependent on the presence of the target nucleic acid, the detection of the signal is successfully done at the predetermined temperature by adjusting the Tₘ value of the duplex. For example, where the signal is generated by the PTOCE method, the detection of the signal is successfully done at the predetermined temperature by adjusting the Tₘ value of the extended duplex formed by the extension of the PTO fragment on the CTO.

The PTOCE-based methods have advantages to readily adjust Tₘ values of the duplex or a third hybrid whose hybridization is affected by the duplex.

According to an embodiment of this invention, when the oligonucleotide set generates a signal in a dependent manner on cleavage of a signaling oligonucleotide, the detection temperature is arbitrarily selected. In other words, any temperature can be selected so long as the signal generated by cleavage of a signaling oligonucleotide may be detected. As described above, where the signal is generated being dependent manner on cleavage of the signaling oligonucleotide, the label released by the cleavage may be detected at any temperature.

According to an embodiment, where the signal is generated being dependent manner on cleavage of the signaling oligonucleotide, the detection temperature is selected to be a relatively highest detection temperature.

As discussed above, the detection temperatures are determined in considering detection temperature ranges depending on oligonucleotide sets. Therefore, the signal detection at a certain detection temperature may be described as follows: the detection at the first detection temperature is to obtain mixed signals from an HPV type having the first detection temperature, an HPV type having the second detection temperature and an HPV type having the third detection temperature, the detection at the second detection temperature is to obtain mixed signals from the HPV type having the second detection temperature and the HPV type having the third detection temperature, and the detection at the third detection temperature is to obtain a signal only from the HPV type having the third detection temperature.

One of the three HPV types has the first detection temperature determined by the corresponding oligonucleotide set, another has the second detection temperature determined by the corresponding oligonucleotide set, and the other has the third detection temperature determined by the corresponding oligonucleotide set.

For instance, where a signal for an HPV type having the first detection temperature, a signal for an HPV type having the second detection temperature, and a signal for an HPV type having the third detection temperature are all generated by the PTOCE method, the signal for the HPV type having the first detection temperature is generated by an extended duplex having a Tₘ value suitable for the first detection temperature, the signal for the HPV type having the second detection temperature is generated by an extended duplex having a Tₘ value suitable for the second detection temperature, and the signal for the HPV type having the third detection temperature is generated by an extended duplex having a Tₘ value suitable for the third detection temperature. For example, when the first detection temperature, the second detection temperature, and the third detection temperature are a low temperature, a middle temperature, and a high temperature, respectively, all of the extended duplex having a Tₘ value suitable for the high detection temperature, the extended duplex having a Tₘ value suitable for the middle detection temperature and the extended duplex having a Tₘ value suitable for the low detection temperature have their duplex form at the low detection temperature, such that mixed signals for the HPV type having the low detection temperature, the HPV type having the middle temperature, and the HPV type having the high detection temperature are detected at the low detection temperature; the extended duplex having a Tₘ value suitable for the high detection temperature and the extended duplex having a Tₘ value suitable for the middle detection temperature have their duplex form while the extended duplex having a Tₘ value suitable for the low detection temperature is dissociated at the middle detection temperature, such that mixed signals for the HPV type having the high detection temperature and the HPV type having the middle detection temperature are detected except for a signal for the HPV type having the low detection temperature at the middle detection temperature; the extended duplex having a Ty value suitable for the high detection temperature has their duplex form while the extended duplex having a Tₘ value suitable for the middle detection temperature and the extended duplex having a Tₘ value suitable for the low detection temperature are dissociated into a single strand at the high detection temperature, such that only the signal for the HPV type having the high detection temperature is detected at the high detection temperature.

In another example, where the signal for the HPV type having the high detection temperature is generated by TaqMan method and the signal for the HPV type having the middle detection temperature and the signal for the HPV type having the low detection temperature are generated by the PTOCE method, the signal for the HPV type having the high detection temperature is provided by a released fluorescent label and the signal for the HPV type having the middle detection temperature and the signal for the HPV type having the low detection temperature are provided by an extended duplex having a Tₘ value suitable for the middle detection temperature and an extended duplex having a Tₘ value suitable for the low detection temperature, respectively. In this case, at the high detection temperature, the extended duplex having a Tₘ value suitable for the middle detection temperature and the extended duplex having a Ty value suitable for the low detection temperature are dissociated to be in a single strand, such that only the signal from the released fluorescent label for the HPV type having the high detection temperature is detected at the high detection temperature. At the middle detection temperature, not only the signal provided from the extended duplex having a Tₘ value suitable for the middle detection temperature but also the signal from the released fluorescent label are detected, such that mixed signals for the HPV type having the middle detection temperature and the HPV type having the high detection temperature are detected. At low detection temperature, all of the signal provided from the extended duplex having a Tₘ value suitable for the low temperature, the signal provided from the extended duplex having a Tₘ value suitable for the low temperature, and the signal from the released fluorescent label are detected, such that mixed signals for the HPV type having the low detection temperature, the HPV having the middle detection temperature, and the HPV having the high detection temperature.

In one embodiment, the first detection temperature is a temperature selected from 57 to 63° C., the second detection temperature is a temperature selected from 69 to 75° C., and the third detection temperature is a temperature selected from 80 to 86° C. In a particular embodiment, the first detection temperature is 60° C., the second detection temperature is 72° C., and the third detection temperature is 83° C.

Such detection temperatures may be used in case that all of the at least nine oligonucleotide sets generate signals by cleavage of the mediation oligonucleotide hybridized with a target nucleic acid and then formation or dissociation of a duplex between the cleavage product and the signaling oligonucleotide, which occurs dependently on the presence of the target nucleic acid, or in case that an oligonucleotide set for the target nucleic acid having the third detection temperature generates a signal by hybridization of the signaling oligonucleotide with the target nucleic acid and then cleavage of the signaling oligoncucleotide, which occurs dependently on the presence of the target nucleic acid and both oligonucleotide sets for the target nucleic acid having the first detection temperature and the target nucleic acid having the second detection temperature generate signals by cleavage of the mediation oligonucleotide hybridized with a target nucleic acid and then formation or dissociation of a duplex between the cleavage product and the signaling oligonucleotide, which occurs dependently on the presence of the target nucleic acid.

In another embodiment, the first detection temperature can be selected from 80° C. to 86° C., the second detection temperature can be selected from 69° C. to 75° C. and the third detection temperature can be selected from 57° C. to 63° C. In certain embodiments, the first detection temperature is 83° C., the second detection temperature is 72° C. and the third detection temperature is 60° C.

Such detection temperatures may be used in case that all of the at least nine oligonucleotide sets generate signals by cleavage of the signaling oligonucleotide hybridized with a target nucleic acid and then dissociation of a duplex, which occurs dependently on the presence of the target nucleic acid, or in case that an oligonucleotide set for the target nucleic acid having a third detection temperature generates a signal by hybridization of the signaling oligonucleotide with the target nucleic acid and then cleavage of the signaling oligonucleotide, which occurs dependently on the presence of the target nucleic acid and both oligonucleotide sets for the target nucleic acid having the first detection temperature and the target nucleic acid having the second detection temperature generate signals by cleavage of the signaling oligonucleotide hybridized with the target nucleic acid and then dissociation of a duplex, which occurs dependently on the presence of the target nucleic acid.

The detection channel used in the present invention includes any means capable of detecting signals. For example, the detection channel may be a photodiode capable of detecting a fluorescent signal of a specific wavelength.

According to one embodiment, the generation of a signal includes “signal generation or extinguishment” and “signal increase or decrease” from labels. Specifically, the generation of a signal means the generation of a significant signal. The significant signal means a signal that can be distinguished from a background signal or from a signal derived from a reaction using a sample not containing the target nucleic acid (i.e., negative sample). That is, when the intensity of a generated signal is equal to or close to the intensity of a background signal or the intensity of a signal derived from a reaction using a negative sample, it is considered that no signal is generated for the sample of the interest.

The method of the present invention is characterized in that high concentrations of oligonucleotides are used to detect target nucleic acids of at least nine HPV types.

There is no prior art teaching a method of distinguishably determining the presence of at least nine, in particular fourteen (14) HPV types and 1 internal control in a single reaction by real-time PCR. Conventional methods of measuring a signal at one detection temperature per detection channel can obtain a signal of a relatively high signal intensity for a target nucleic acid by selection of a suitable detection temperature.

In contrast, since the method of the present invention measures signals at three detection temperatures having a predetermined interval per detection channel, some of the three detection temperatures are inevitably higher than the detection temperature used in the conventional methods. It is known in the art that oligonucleotides and fluorescent labels tend to produce a signal of low intensity at higher detection temperatures, which may lead to a false negative result due to low signal intensity. To compensate for the low signal intensity, the method of the present invention employs a high concentration of oligonucleotides.

According to one embodiment, the total concentration of the amplifying oligonucleotides in the reaction vessel is 15 to 60 pmole/μL (uM).

The total concentration of the amplifying oligonucleotides may vary depending on the number of oligonucleotide sets used in the present invention. In certain embodiments, the total concentration of the amplifying oligonucleotides is 15 to 55, 15 to 50, 15 to 45, 15 to 40, 15 to 35, 20 to 55, 20 to 50, 20 to 45, 20 to 40, 20 to 35, 25 to 55, 25 to 50, 25 to 45, 25 to 40, 25 to 35, 30 to 55, 30 to 50, 30 to 45, 30 to 40, 30 to 35, 35 to 55, 35 to 50, 35 to 45, 35 to 40, 40 to 55, 40 to 50, 40 to 45, 45 to 55, 45 to 50, or 50 to 55 pmole/uL.

In one embodiment, when nine (9) oligonucleotide sets are used, the total concentration of the amplifying oligonucleotides in the reaction vessel is 15 to 30, 15 to 35, 15 to 40, 20 to 30, 20 to 35, 20 to 40, 25 to 30, 25 to 35, 25 to 40, 30 to 35, or 35 to pmole/uL.

In another embodiment, when twelve (12) oligonucleotide sets are used, the total concentration of the amplifying oligonucleotides in the reaction vessel is 25 to 40, 25 to 35, 25 to 30, 30 to 40, 30 to 35, or 35 to 40 pmole/uL.

In another embodiment, when fifteen (15) oligonucleotide sets are used, the total concentration of the amplifying oligonucleotides in the reaction vessel is 35 to 60, 35 to 55, 35 to 50, 35 to 40, 40 to 60, 40 to 55, 40 to 50, 40 to 45, 45 to 60, 45 to 55, 45 to 50, 50 to 60, or 50 to 55 pmole/uL.

When the amplifying oligonucleotides are composed of a forward primer and a reverse primer, the concentration of the forward primer may or may not be equal to that of the reverse primer.

According to the invention, the total reaction volume in the reaction vessel is 10 to μL. In certain embodiments, the total reaction volume in the reaction vessel is 20 μL.

The total amount of the amplifying oligonucleotides in the reaction vessel can be calculated by multiplying the total concentration of the amplifying oligonucleotides described above by the total reaction volume described above.

According to the invention, the total concentration of the signaling oligonucleotides is from 8 to 35 pmole/uL.

The total concentration of the signaling oligonucleotides may vary depending on the number of oligonucleotide sets used in the present invention. In certain embodiments, the total concentration of the signaling oligonucleotides is 8 to 30, 8 to 25, 8 to 20, 8 to 15, 8 to 10, 15 to 30, 15 to 25, 15 to 20, 20 to 30, 2 to 25, or 25 to 30 pmole/uL.

In one embodiment, when nine (9) oligonucleotide sets are used, the total concentration of the signaling oligonucleotides in the reaction vessel is 8 to 15, 8 to 10, or to 15 pmole/uL.

In another embodiment, when twelve (12) oligonucleotide sets are used, the total concentration of the signaling oligonucleotides in the reaction vessel is 15 to 25, 15 to 20, or 20 to 25 pmole/uL.

In another embodiment, when fifteen (15) oligonucleotide sets are used, the total concentration of the signaling oligonucleotides in the reaction vessel is 20 to 35, 20 to 30, 20 to 25, 25 to 35, 25 to 30, or 30 to 35 pmole/uL.

When the signaling oligonucleotides are composed of two or more oligonucleotides, the concentration of each of these oligonucleotides may or may not be equal to each other.

The total amount of the signaling oligonucleotides in the reaction vessel can be calculated by multiplying the total concentration of the signaling oligonucleotides described above by the total reaction volume described above.

The total concentration of oligonucleotides used in the method of the present invention may be from 23 to 95 pmole/uL. In one embodiment, the total concentration of oligonucleotides used in the method of the present invention is 30 to 90, 30 to 80, 30 to 70, 30 to 60, 30 to 50, 30 to 40, 40 to 90, 40 to 80, 40 to 70, 40 to 60, 40 to 50, 50 to 90, 50 to 80, 50 to 70, 50 to 60, 60 to 90, 60 to 80, 60 to 70, 70 to 90, 70 to 80, or 80 to pmole/uL.

The method of the present invention is characterized that a large number of oligonucleotides are used to detect target nucleic acids of at least nine HPV types.

Conventional methods based on screening cannot identify HPV types or which HPV type is present in a sample, due to the limitation of available fluorescent labels. That is, as conventional methods screen all HPV types using one oligonucleotide set, they do not require a large number of oligonucleotides.

In contrast, the present method uses at least nine oligonucleotide sets, each specific for each HPV type to identify at least nine HPV types, and therefore requires a large number of oligonucleotides in one reaction.

In one embodiment, 3 to 10 oligonucleotides are included per each oligonucleotide set. In certain embodiments, 3 to 4 oligonucleotides are included per each oligonucleotide set.

According to the invention, the total number of the amplifying oligonucleotides in the reaction vessel is from 18 to 40.

The total number of the amplifying oligonucleotides may vary depending on the number of oligonucleotide sets used in the present invention. In certain embodiments, the total number of the amplifying oligonucleotides is 18 to 35, 18 to 30, 18 to 25, 18 to 20, 20 to 35, 20 to 30, 20 to 25, 25 to 35, 25 to 30, 25 to 30, or 30 to 35.

According to the invention, the total number of the signaling oligonucleotides in the reaction vessel is from 18 to 40.

The total number of signaling oligonucleotides may vary depending on the number of oligonucleotide sets used in the present invention. In certain embodiments, the total number of the signaling oligonucleotides is 18 to 35, 18 to 30, 18 to 25, 18 to 20, 20 to 35, 20 to 30, 20 to 25, 25 to 35, 25 to 30, 25 to 30, or 30 to 35.

The total number of oligonucleotides used in the present invention is from 36 to 80. In one embodiment, the total number of oligonucleotides used in the present invention is 36 to 75, 36 to 70, 36 to 65, 36 to 60, 36 to 55, 36 to 50, 36 to 45, 36 to 40, 48 to 75, to 70, 48 to 65, 48 to 60, 48 to 55, 48 to 50, 60 to 75, 60 to 70, 60 to 70, or 60 to 65. In certain embodiments, the number of oligonucleotides used in the present invention is at least 36. In other specific embodiments, the number of oligonucleotides used in the present invention is at least 45.

### Step (b): Measurement of Signals at Three Temperatures

In this step, signals are measured at the first detection temperature, the second detection temperature, and the third detection temperature using the at least three detection channels.

According to the method of the present invention, at least three detection channels are used to detect target nucleic acids of at least nine HPV types. The number of detection channels used is equal to the number of types of fluorescent labels included in the at least nine oligonucleotide sets of step (a). As one example, if among nine oligonucleotide sets used in step (a), three oligonucleotide sets include a first fluorescent label, another three oligonucleotide sets include a second fluorescent label, and the other three oligonucleotide sets include a third fluorescent label, three detection channels are used in step (b) to detect signals from the three fluorescent labels. As another example, if among fifteen (15) oligonucleotide sets used in step (a), five different types of fluorescent labels are included therein, five detection channels are used in step (b).

The detection channel is for detecting a signal from one fluorescent label. For example, a signal generated from an FAM fluorescent label is detected by an FAM detection channel, and a signal generated from a CAL Fluor Orange 560 fluorescent label is detected by a CAL Fluor Orange 560 detection channel.

According to the method of the present invention, signals are measured at the first detection temperature, the second detection temperature, and the third detection temperature. The first detection temperature, the second detection temperature and the third detection temperature are described elsewhere herein.

In particular embodiments, signals are measured at detection temperatures of 60° C., 72° C., and 83° C. using a detection channel for FAM or an equivalent thereof, signals are measured at detection temperatures of 60° C., 72° C., and 83° C. using a detection channel for CAL Fluor Orange 560 or an equivalent thereof, signals are measured at detection temperatures of 60° C., 72° C., and 83° C. using a detection channel for CAL Fluor Red or an equivalent thereof, signals are measured at detection temperatures of 60° C., 72° C., and 83° C. using a detection channel for ATTO 647N or an equivalent thereof, and signals are measured at detection temperatures of 60° C., 72° C., and 83° C. using a detection channel for ATTO 680 or an equivalent thereof.

Measurement of the signals at the three detection temperatures may be performed at each cycle of amplification. For example, nucleic acid amplification may be performed for 40, 45 or 50 cycles of PCR, and signals may be measured at three detection temperatures for each cycle.

In one embodiment, the measurement of the signals at the three detection temperatures produces three amplification curves (a set of data points consisting of an RFU and a cycle): an amplification curve at the first detection temperature, an amplification curve at the second detection temperature, and an amplification curve at the third detection temperature.

According to the present invention, at least nine oligonucleotide sets are used to obtain at least nine amplification curves (amplification curves at three detection temperatures per one detection channel).

### Step (c): Determination of Presence of Target Nucleic Acids

In this step, the presence of target nucleic acids of the at least nine HPV types is determined by the signals measured at the first detection temperature, the second detection temperature and the third detection temperature using each of the at least three detection channels.

Specifically, the presence of the first target nucleic acid can be determined by analyzing the signals measured at the first detection temperature, the second detection temperature, and the third detection temperature; the presence of the second target nucleic acid can be determined by analyzing the signals measured at the second detection temperature and the third detection temperature. Further, the presence of the third target nucleic acid can be determined from the signal measured at the third detection temperature.

As described above, the first oligonucleotide set generates a signal at the first detection temperature in the presence of a target nucleic acid of a corresponding HPV type, the second oligonucleotide set generates a signal at the first detection temperature and the second detection temperature in the presence of a target nucleic acid of a corresponding HPV type, and the third oligonucleotide set generates a signal at the first detection temperature, the second detection temperature and the third detection temperature in the presence of a target nucleic acid of a corresponding HPV type.

Thus, in view of the relationship between a target nucleic acid of each HPV type and a corresponding oligonucleotide set, depending on whether each target nucleic acid is present in the sample, the signal measured at the first detection temperature includes any one selected from the group consisting of a signal generated by the first oligonucleotide set at the first detection temperature, a signal generated by the second oligonucleotide set at the first detection temperature, a signal generated by the third oligonucleotide set at the first detection temperature, and combinations thereof. In this case, the signal generated by each oligonucleotide set means a signal of significance.

As an example, if the sample contains a first target nucleic acid, does not contain a second target nucleic acid, and does not contain a third target nucleic acid, the signal measured at the first detection temperature includes a signal generated by the first oligonucleotide set, does not include a signal generated by the second oligonucleotide set, and does not include a signal generated by the third oligonucleotide set.

As another example, if the sample does not contain a first target nucleic acid, contains a second target nucleic acid, and does not contain a third target nucleic acid, the signal measured at the first detection temperature does not include a signal generated by the first oligonucleotide set, includes a signal generated by the second oligonucleotide set, and does not include a signal generated by the third oligonucleotide set.

As another example, if the sample does not contain a first target nucleic acid, does not contain a second target nucleic acid, and contains a third target nucleic acid, the signal measured at the first detection temperature does not include a signal generated by the first oligonucleotide set, does not include a signal generated by the second oligonucleotide set, and includes a signal generated by the third oligonucleotide set.

As another example, if the sample contains a first target nucleic acid, contains a second target nucleic acid, and does not contain a third target nucleic acid, the signal measured at the first detection temperature includes a signal generated by the first oligonucleotide set, includes a signal generated by the second oligonucleotide set, and does not include a signal generated by the third oligonucleotide set.

As another example, if the sample does not contain a first target nucleic acid, contains a second target nucleic acid, and contains a third target nucleic acid, the signal measured at the first detection temperature does not include a signal generated by the first oligonucleotide set, includes a signal generated by the second oligonucleotide set, and includes a signal generated by the third oligonucleotide set.

As another example, if the sample contains a first target nucleic acid, does not contain a second target nucleic acid, and contains a third target nucleic acid, the signal measured at the first detection temperature includes a signal generated by the first oligonucleotide set, does not include a signal generated by the second oligonucleotide set, and includes a signal generated by the third oligonucleotide set.

As another example, if the sample contains a first target nucleic acid, a second target nucleic acid, and a third target nucleic acid, the signal measured at the first detection temperature includes a signal generated by the first oligonucleotide set, a signal generated by the second oligonucleotide set, and a signal generated by the third oligonucleotide set.

As another example, if the sample does not contain a first target nucleic acid, a second target nucleic acid, and a third target nucleic acid, the signal measured at the first detection temperature does not include a signal generated by the first oligonucleotide set, a signal generated by the second oligonucleotide set, and a signal generated by the third oligonucleotide set. That is, in this case, no signal is measured at the first detection temperature. Therefore, if a significant signal is not measured at the first detection temperature, it can be determined that the first target nucleic acid, the second target nucleic acid, and the third target nucleic acid are all not present in the sample.

In addition, in view of the relationship between a target nucleic acid of each HPV type and a corresponding oligonucleotide set, depending on whether each target nucleic acid is included in the sample, the signal measured at the second detection temperature includes any one selected from the group consisting of a signal generated by the second oligonucleotide set at the second detection temperature, a signal generated by the third oligonucleotide set at the second detection temperature, and a combination thereof.

In other words, if the sample does not contain a first target nucleic acid, a second target nucleic acid, and a third target nucleic acid, or if the sample contains a first target nucleic acid, does not contain a second target nucleic acid, and does not contain a third target nucleic acid, no signal is measured at the second detection temperature. Therefore, if a significant signal is not measured at the second detection temperature, it can be determined that the second target nucleic acid and the third target nucleic acid are not present in the sample.

In the present method, since the signal generated by the first oligonucleotide set is induced by interaction between the first oligonucleotide set and the first target nucleic acid, the signal may be referred to as a signal generated by the first target nucleic acid, a signal generated from the first target nucleic acid, a signal generated in the presence of the first target nucleic acid, a signal provided by the first target nucleic acid, or a signal for the first target nucleic acid. This applies mutatis mutandis to the signal generated by the second oligonucleotide set or the third oligonucleotide set.

It is to be understood that the meaning of the signal generated by the oligonucleotide set is different from that of the signal measured at the first detection temperature, the second detection temperature or the third detection temperature. As described above, the signal measured at each detection temperature may include a combination of indistinguishable signals for multiple targets, while the signal generated by the oligonucleotide set is a signal for only one target nucleic acid.

In one embodiment, in step (c), the presence of the first target nucleic acid can be determined by differences between two signals selected from the signals measured at the first detection temperature, the second detection temperature, and the third detection temperature, the presence of the second target nucleic acid can be determined by a difference between the signals measured at the second detection temperature and the third detection temperature, and the presence of the third target nucleic acid can be determined by the signal measured at the third detection temperature.

The signals used for determination of the presence of a target nucleic acid includes various signal characteristics from the signal detection, e.g., signal intensity [e.g., RFU (relative fluorescence unit) value or in the case of performing amplification, RFU values at a certain cycle, at selected cycles or at end-point], signal change shape (or pattern) or Ct value, or values obtained by mathematically processing the characteristics.

According to an embodiment of this invention, when an amplification curve is obtained by real-time PCR, various signal values (or characteristics) from the amplification curve may be selected and used for determination of the presence of a target nucleic acid, including intensity, Ct value or amplification curve data.

The characteristics of the signal obtained at each detection temperature per se may be used to determine the presence of each target nucleic acid.

Alternatively, a modified signal provided by mathematically processing the characteristics of the signal may be used to determine the presence of the third target nucleic acid.

The characteristics of the signal measured at the first detection temperature per se, the characteristics of the signal measured at the second detection temperature per se, and the characteristics of the signal measured at the third detection temperature per se may be used to obtain the differences between the signals at the detection temperatures.

Alternatively, one or more of the signals at the first detection temperature, the second detection temperature and the third detection temperature may be modified by mathematically processing the characteristics of the signal and used to obtain the difference between the signals at two detection temperatures among the first detection temperature, the second detection temperature, and the third detection temperature.

According to an embodiment, the term “signal” with conjunction with the phrase “signals measured at a first detection temperature, a second detection temperature, and/or a third detection temperature” includes not only a signal measured at the detection temperature per se but also a modified signal provided by mathematically processing the signal.

According to an embodiment, where the mathematical processing is done, the characteristics of the signal should be characteristics vulnerable to the mathematical processing. In certain embodiments, the mathematical processing includes calculation (e.g., addition, multiplication, subtraction and division) using signals or obtaining other values derived from signals. The signals used for determination of the presence of target nucleic acids in the present invention generally are significant signals. In other words, the signals are those generated dependently on the presence of a target nucleic acid. In the meantime, where the difference between the signals detected at two detection temperatures among the first detection temperature, the second detection temperature, and the third detection temperature is calculated, a signal of insignificance such as background signals may be used to calculate the difference. In this regard, it would be understood that the signals used for determination of the presence of target nucleic acids encompass not only signals of significance but also signals of insignificance so long as they can be used to calculate the difference or involved in a determination process.

According to an embodiment, significance of signals detected may be determined using a threshold value. For example, a threshold value is predetermined from a negative control in considering background signals of detector, sensitivity or label used, and then the significance of signals from samples may be determined.

Where a signal (i.e., a significant signal) is detected at the third detection temperature, it is determined that the target nucleic acid having the third detection temperature is present.

The signal of insignificance may be also expressed herein by “absence of signal” or “no detection of signal”.

The term used herein “by a signal” with conjunction to determination of the presence of target nucleic acid means that the presence of target nucleic acid is determined by directly or indirectly using or modifying signals generated from the oligonucleotide sets, including using numerical values of signals or their modifications, using the presence/absence of signals and comparing the signal with a threshold.

The term used herein “determination by a signal” with reference to determination of the presence of the target nucleic acid may include determining the presence of the third target nucleic acid with considering significance of the signal detected at the third detection temperature. It may also include determining the absence of the second target nucleic acid and the third target nucleic acid with considering significance of the signal detected at the second detection temperature. Further, it may include determining the absence of the first target nucleic acid, the second target nucleic acid, and the third target nucleic acid with considering significance of the signal detected at the first detection temperature.

In this invention, the presence of the first target nucleic acid is determined by analyzing the signals measured at the first detection temperature, the second detection temperature, and the third detection temperature. Further, the presence of the second target nucleic acid is determined by analyzing the signals measured at the second detection temperature and the third detection temperature.

Where signals are detected at the first detection temperature and the second detection temperature, said signals per se do not permit to determine the presence of the first target nucleic acid and/or the presence of the second target nucleic acid. The reason for those is that the signal for the third target nucleic acid may be also measured at the first detection temperature and the second detection temperature, and the signal for the second target nucleic acid may be also measured at the first detection temperature.

The feature of the present invention is to employ the signals measured at the second detection temperature and the third detection temperature for analyzing the signal measured at the first detection temperature, and to employ the signal measured at the third detection temperature for analyzing the signal measured at the second detection temperature.

Interestingly, the present inventors have found that when signals indicating the presence of a single target nucleic acid are detected in a single reaction vessel at predetermined three detection temperatures, there is a signal change in a certain pattern (rule).

For example, for the first, second, or third target nucleic acid, a signal change (change in signal intensity) between a signal measured at the third detection temperature and a signal measured at the second detection temperature, a signal change between a signal measured at the third detection temperature and a signal measured at the first detection temperature, and a signal change between a signal measured at the second detection temperature and a signal measured at the first detection temperature shows a certain pattern (rule). For example, the intensities of the signals at two detection temperatures selected from the first, second, and third detection temperatures may be identical or substantially identical to each other or the intensities of the signals may be different from each other but in a certain range.

The feature of the present invention is to adopt the findings to detection of target nucleic acids.

Because signals for a target nucleic acid in a single reaction vessel are detected with differing only detection temperatures (e.g., no change of amount of the target or no variation of buffer conditions), there is a certain pattern (rule) in a signal change between the two detection temperatures. Based on the certain pattern (rule) in the signal change, the signal measured at the second detection temperature and the signal measured at the third detection temperature can be used for analyzing the signal measured at the first detection temperature, and the signal measured at the third detection temperature can be used for analyzing the signal measured at the second detection temperature.

According to an embodiment, the present method is performed under a condition that permits a certain pattern (rule) in a signal change between two detection temperatures for a target nucleic acid.

According to an embodiment, the presence of the second target nucleic acid is determined in such a manner that the signal measured at the second detection temperature is analyzed by using the signal measured at the third detection temperature in order to verify whether the signal measured at the second detection temperature contains a signal provided by the second target nucleic acid.

According to an embodiment, the presence of the first target nucleic acid is determined in such a manner that the signal measured at the first detection temperature is analyzed by using the signal measured at the third detection temperature and the signal measured at the second detection temperature in order to verify whether the signal measured at the first detection temperature contains a signal provided by the first target nucleic acid.

The analysis of the signal measured at the second detection temperature by using the signal measured at the third detection temperature may be conducted by obtaining a difference between the signal measured at the third detection temperature and the signal measured at the second detection temperature and then analyzing it.

According to an embodiment of this invention, the extent (or portion) of signal of the second target nucleic acid in the signal measured at the second detection temperature may be obtained under the principle by using the signal measured at the third detection temperature. Further, the analysis of the signal measured at the first detection temperature by using the signal measured at the third detection temperature and the signal measured at the second detection temperature may be conducted by obtaining some of differences between two signals selected from the signal measured at the third detection temperature, the signal measured at the second detection temperature, and the signal measured at the first detection temperature, and then analyzing them.

According to an embodiment of this invention, the extent (or portion) of signal of the second target nucleic acid in the signal measured at the second detection temperature may be obtained under the principle by using the signal measured at the third detection temperature. Further, the extent (or portion) of signal of the first target nucleic acid in the signal measured at the first detection temperature may be obtained under the principle by using the signal detected at the third detection temperature and the signal measured at the second detection temperature.

According to an embodiment, the presence of the second target nucleic acid may be determined by a difference between the signal measured at the third detection temperature and the signal measured at the second detection temperature, and the presence of the first target nucleic acid may be determined by differences between two signals selected from the signals measured at the first detection temperature, the second detection temperature and the third detection temperature.

For example, if the sample contains a first target nucleic acid, does not contain a second target nucleic acid, and does not contain a third target nucleic acid, a signal is measured at the first detection temperature, but not at the second detection temperature and the third detection temperature. No signal detection at the second detection temperature and the third detection temperature indicates the absence of the second target nucleic acid and the third target nucleic acid, such that the signal measured at the first detection temperature can be recognized to be due to the first target nucleic acid, whereby the presence of the first target nucleic acid can be determined.

Alternatively, in this case, the difference may be obtained by using signal of insignificance (e.g., background signal) measured at the second or third detection temperature. In this alternative, the difference is very likely to be distinctly different from the difference in other cases below, whereby the presence of the first target nucleic acid can be determined.

In addition, if the sample does not contain a first target nucleic acid, contains a second target nucleic acid, and does not contain a third target nucleic acid, signals are detected at the first detection temperature and the second detection temperature, but not at the third detection temperature. The signal measured at the second detection temperature is likely to be different from that measured at the first detection temperature. Such difference is very likely to be within a certain range because all conditions except for detection temperatures are common. Where the difference calculated for a sample falls within the certain range, the signal detected at the first detection temperature and the signal detected at the second detection temperature is due to only the second target nucleic acid. In other words, the first target nucleic acid and the third target nucleic acid can be determined to be absent in the sample.

In addition, if the sample does not contain a first target nucleic acid, does not contain a second target nucleic acid, and contains a third target nucleic acid, signals are measured at the first, second and third detection temperatures. The signal measured at the third temperature is likely to be different from that measured at the first detection temperature and/or that measured at the second detection temperature. Such difference is very likely to be within a certain range because all conditions except for detection temperatures are common. Where the difference calculated for a sample falls within the certain range, the signals measured at the first and second detection temperatures are due to only the third target nucleic acid. In other words, the first and second target nucleic acids can be determined to be absent in the sample.

In addition, if the sample contains a first target nucleic acid, contains a second target nucleic acid, and does not contain a third target nucleic acid, signals are measured at the first detection temperature and the second detection temperature, but not at the third detection temperature. The difference between the signals at the first detection temperature and the second detection temperature become more distinguishable than the difference where only the second target nucleic acid is present, because the first target nucleic acid is present. The presence of the first target nucleic acid can be determined using the difference between the signals at the first detection temperature and the second detection temperature.

In addition, if the sample does not contain a first target nucleic acid, contains a second target nucleic acid, and contains a third target nucleic acid, signals can be significantly measured at the first detection temperature, the second detection temperature and the third detection temperature. The difference between the signals at the second detection temperature and the third detection temperature become more distinguishable than the difference where only the third target nucleic acid is present, because the second target nucleic acid is present. On the other hand, since the second target nucleic acid and the third target nucleic acid generate signals at both the first detection temperature and the second detection temperature, the difference of the signals between the second detection temperature and the third detection temperature can be applied to not only the determination of the presence of the second target nucleic acid, but also the determination of the first target nucleic acid using the difference between the signals measured at the first and second detection temperatures because the second and third target nucleic acids generate signals at the first and second detection temperatures. Thus, the absence of the first target nucleic acid can be determined by using the above difference.

In addition, if the sample contains a first target nucleic acid, does not contain a second target nucleic acid, and contains a third target nucleic acid, signals can be significantly measured at the first detection temperature, the second detection temperature and the third detection temperature. The difference between the signals at the first detection temperature and the third detection temperature become more distinguishable than the difference where only the third target nucleic acid is present, because the first target nucleic acid is present. On the other hand, because the first and third target nucleic acids generate signals at the first detection temperatures, the difference between the signal measured at the first and third detection temperatures is applied to not only the determination of the second target nucleic acid, but also the determination of the first target nucleic acid using the difference between the signal measured at the first and second detection temperatures because the second and third target nucleic acids generate signals at the first and second detection temperatures. Thus, the presence of the first target nucleic acid can be determined by using the above difference.

Further, if the sample contains a first target nucleic acid, a second target nucleic acid, and a third target nucleic acid, signals can be significantly measured at the first detection temperature, the second detection temperature and the third detection temperature. The presence of the first target nucleic acid and the second nucleic acid can be determined using the differences.

The differences between two signals selected from signals measured at the first detection temperature, the second detection temperature and the third detection temperature may be obtained in accordance with a wide variety of approaches.

The term used herein “difference” with conjunction to “by (or using) the difference between the signals” includes not only a difference to be obtained by mathematically processing signals per se or modified signals but also a difference due to the presence and absence of signals. For example, the difference may be obtained by calculating the ratio or subtraction between two signals selected from the signal measured at the first detection temperature, the signal detected at the second detection temperature and the signal detected at the third detection temperature. Alternatively, the difference may be given by modifying a signal at one detection temperature and comparing it with a signal at other detection temperature. The difference between two signals selected from the signal detected at the first detection temperature, the signal detected at the second detection temperature and the signal detected at the third detection temperature may be expressed in various aspects. For example, the difference may be expressed as numerical values, the presence/absence of signal or plot with signal characteristics.

According to an embodiment of this invention, the difference between two signals selected from the signal measured at the first detection temperature, the signal detected at the second detection temperature and the signal detected at the third detection temperature comprises a difference to be obtained by mathematically processing the two signals.

According to an embodiment of this invention, when no signal is detected at the third detection temperature, the determination of the presence of the first target nucleic acid and the presence of the second target nucleic acid is made by the signals measured at the first detection temperature and the second detection temperature with considering no detection of the signal at the third detection temperature. Further, when no signal is measured at the second detection temperature, the determination of the presence of the first target nucleic acid is made by the signal measured at the first detection temperature with considering no detection of the signal at the second detection temperature. This embodiment addresses that using a difference due to the presence and absence of signals at each detection temperature allows for the determination of the presence of the first target nucleic acid.

According to an embodiment, a background signal measured at the second detection temperature, or a background signal measured at the third detection temperature may be treated as “0” or “1” for calculating the difference.

According to an embodiment, where a minus value is obtained during calculation, it is converted to absolute value and used to obtain the difference.

According to an embodiment of this invention, the signal measured at the third detection temperature is a calculation parameter to analyze the signal for the first target nucleic acid and the signal for the second target nucleic acid. Further, the signal measured at the second detection temperature is a calculating parameter to analyze the signal for the first target nucleic acid. That is, two or more parameters are required to analyze the signal for the first target nucleic acid.

The signals for determining the presence of the third target nucleic acid and the differences between two signals selected from the signal detected at the first detection temperature, the signal detected at the second detection temperature and the signal detected at the third detection temperature may have different dimensions or units from each other or have same dimensions or units from each other.

The term used herein “determined by a difference” includes determined by occurrence/non-occurrence of a difference, determined by value or range of a difference with a numerical value and determined by a plotting result of the difference. Furthermore, “determined by a difference” includes obtaining values (e.g., CT) for the first target nucleic acid and the second target nucleic acid on the basis of the differences.

The term used herein “by a difference” with conjunction to determination of the presence of target nucleic acids means that the presence of target nucleic acids is determined by directly or indirectly using or modifying a difference between signals, including using numerical values of a difference or its modifications, using the presence/absence of signals and comparing a difference with a threshold. There is no intended distinction between the terms “by a difference” and “by using a difference”, and these terms will be used interchangeably.

The mathematical processing of the signals may be carried out by various calculation methods and their modifications.

According to an embodiment of this invention, the mathematical processing of the signals to obtain the difference between the signals is a calculation of a ratio of the signal measured at the second detection temperature to the signal measured at the third detection temperature, a ratio of the signal measured at the first detection temperature to the signal measured at the third detection temperature, and/or a ratio of the signal measured at the first detection temperature to the signal measured at the second detection temperature

The term used herein “ratio” means a relationship between two numbers. By using the ratio, the presence of the first target nucleic acid and the second target nucleic acid may be determined. Where the ratio of the signal measured at the second detection temperature to the signal measured at the third detection temperature is significant, it becomes entitled as an indicator for the presence of the second target nucleic acid. Where at least one of the ratio of the signal measured at the first detection temperature to the signal measured at the third detection temperature, the ratio of the signal measured at the second detection temperature to the signal measured at the third detection temperature, and the ratio of the signal measured at the first detection temperature to the signal measured at the second detection temperature is significant, it becomes entitled to an indicator for the presence of the first target nucleic acid.

For instance, where the ratio of the end-point intensity of the signal measured at the second detection temperature to the end-point intensity of the signal measured at the third detection temperature is significant (e.g., increase in the end-point intensity), it indicates the presence of the second target nucleic acid. Further, where the ratio of the end-point intensity of the signal measured at the second detection temperature to the end-point intensity of the signal measured at the third detection temperature and the ratio of the end-point intensity of the signal measured at the first detection temperature to the end-point intensity of the signal measured at the second detection temperature are significant, it indicates the presence of the second target nucleic acid and the first target nucleic acid.

The mathematical processing may be carried out in various fashions.

The mathematical processing may be carried out by use of a machine. For example, the signals may be undergone a mathematical processing by a processor in a detector or real-time PCR device. Alternatively, the signals may be manually undergone a mathematical processing particularly according to a predetermined algorithm.

According to an embodiment of this invention, depending on approaches for obtaining the difference, a threshold may be employed to analyze whether the differences obtained are indicative of the presence of the first target nucleic acid and the second target nucleic acid. For example, a threshold is predetermined with considering the difference obtained from a standard sample containing the first target nucleic acid, the second target nucleic acid and the third target nucleic acid. A negative control, sensitivity or label used may be further considered for determining the threshold.

According to an embodiment of this invention, depending on approaches for obtaining the difference, the presence of the first target nucleic acid and/or the second target nucleic acid may be determined by using the difference obtained per se. For example, a signal at the third detection temperature may be multiplied by a threshold and then the difference between the multiplied signal and a signal at the second detection temperature may be obtained. Further, a signal at the third detection temperature may be multiplied by a threshold, a signal at the second detection temperature may be multiplied by a threshold, and then the difference between the multiplied signals and a signal at the first detection temperature may be obtained. Particularly, the thresholds are predetermined with considering the difference obtained from a standard sample containing the first target nucleic acid, the second target nucleic acid and the third detection temperature.

According to an embodiment of this invention, a threshold is determined by user or automatically.

In an embodiment, where the difference between two signals selected from signals at the first detection temperature, at the second detection temperatures and the third detection temperature for the third target nucleic acid becomes greater, it is more likely to reduce detection errors by using the threshold.

In an embodiment, where signals provided by the third target nucleic acid have a pattern (or rule) showing little or no difference between two detection temperatures selected from the first detection temperature, the second detection temperature and the third detection temperature, the signal measured at the second detection temperature or the signal measured at the third detection temperature may be used without further modifications in either calculation of the difference or determination of the presence of the first target nucleic acid and the second target nucleic acid using the difference.

In certain embodiments, where signals have a pattern (or rule) showing difference within a certain range, the signal at the third detection temperature and the signal at the second detection temperature may be subject to modification reflecting the difference in determination of the presence of the target nucleic acid.

A reference value is a value reflecting a pattern (rule) of a signal change at different temperatures.

According to an embodiment of this invention, the reference value represents a relationship of change in signals at two different detection temperatures selected from the first detection temperature, the second detection temperature, and the third detection temperature, provided by each oligonucleotide set in the presence of each target nucleic acid.

For example, the reference value is any one of a relationship of change in signals at the first detection temperature and the second detection temperature, provided by the first oligonucleotide set, a relationship of change in signals at the first detection temperature and the third detection temperature, provided by the first oligonucleotide set, a relationship of change in signals at the second detection temperature and the third detection temperature, provided by the first oligonucleotide set, a relationship of change in signals at the first detection temperature and the second detection temperature, provided by the second oligonucleotide set, a relationship of change in signals at the first detection temperature and the third detection temperature, provided by the second oligonucleotide set, a relationship of change in signals at the second detection temperature and the third detection temperature, provided by the second oligonucleotide set, a relationship of change in signals at the first detection temperature and the second detection temperature, provided by the third oligonucleotide set, a relationship of change in signals at the first detection temperature and the third detection temperature, provided by the third oligonucleotide set, and a relationship of change in signals at the second detection temperature and the third detection temperature, provided by the third oligonucleotide set.

According to an embodiment of the present invention, the presence of the first target nucleic acid and the second target nucleic acid can be determined using the difference and/or reference value.

For example, where the signals at the third detection temperature and the second detection temperature by the third oligonucleotide set are identical or substantially identical and the extent of difference in the signals at the two detection temperatures is calculated by subtraction of the signals, the reference value is ‘O’ for signals at the two detection temperatures for the third oligonucleotide set. As another example, where the extent of difference in the signals at the two detection temperatures is calculated by division of the signals, the reference value is ‘I’ for signals at the two detection temperatures for the third oligonucleotide set.

In the meantime, where the signals at two detection temperatures for a particular target nucleic acid are different from each other and the extent of difference in the two signals is calculated by subtraction of the signals, the reference value is a positive value or negative value other than ‘O’ for signals at the two detection temperatures for the target nucleic acid. As another example, where the extent of difference in the signals at the two detection temperatures is calculated by division of the signals, the reference value is above or below 1 other than ‘I’ for signals at the two detection temperatures for the target nucleic acid.

In certain embodiments, difference in signals at the second detection temperature and the third detection temperature provided by the third oligonucleotide set in the presence of the third target nucleic acid may be expressed through a reference value.

In certain embodiments, the reference value for the case in which the signals at the second detection temperature and the third detection temperature provided by the third oligonucleotide set in the presence of the third target nucleic acid are different from each other may be different by more than 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 12%, 15%, 20% or 30%, compared with the reference value for the case in which the two signals are the same.

In certain embodiments, the reference value for the third target nucleic acid may be applied to extract the signal for the first nucleic and, and thus it may be used in determination of the presence of the first target nucleic acid, where the reference value for the third oligonucleotide set calculated from the signals at the two detection temperatures is different by more than 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 12%, 15%, 20% or 30% compared with the reference value for the case in which the two signals are the same.

According to one embodiment, the reference value is associated with determination of the presence of the first target nucleic acid and the second target nucleic acid.

According to an embodiment of this invention, the reference value may be employed to analyze whether the difference obtained is indicative of the presence of the first target nucleic acid and/or the second target nucleic acid.

According to an embodiment of this invention, the reference value may be employed to obtain the difference between a signal at the first detection temperature and a signal at the second detection temperature provided by the second oligonucleotide set. Further, the reference value may be employed to obtain the difference between two signals selected from a signal at the first detection temperature, a signal at the second detection temperature and a signal at the third detection temperature.

According to an embodiment of this invention, a reference value is used for determining a threshold. According to an embodiment of this invention, a reference value is used as a threshold with or without a modification of the value. The terms used herein “threshold” and “reference value” for determining the presence of target nucleic acid by analyzing the difference between signals may have the same value or meaning.

Alternatively, where the reference value is employed to obtain the difference between a signal at the third detection temperature and a signal at the second detection temperature, an additional threshold may be used to determine the significance of the difference, i.e., to determine whether the difference indicates the presence of the second target nucleic acid. Further, where the reference value is employed to obtain the differences between two signals selected from a signal at the third detection temperature, a signal at the second detection temperature and a signal at the first detection temperature, additional thresholds may be used to determine the significance of the differences, i.e., to determine whether the differences indicate the presence of the first target nucleic acid.

According to an embodiment of this invention, the method uses a reference value, for determining the presence of the first target nucleic acid and the second target nucleic acid, obtained by (i) incubating one or more of the first target nucleic acid, the second target nucleic acid, and the third target nucleic acid with one or more of the first oligonucleotide set, the second oligonucleotide set, and the third oligonucleotide set in a reaction vessel other than the reaction vessel in the step (a), (ii) measuring signals at two detection temperatures selected from the first detection temperature, the second detection temperature and the third detection temperature, and (iii) then obtaining a difference between the signals measured at the two detection temperatures.

According to an embodiment, the difference between the signals obtained in the above method is a value and the value is used as a reference value with modification or without modification.

For example, the difference between the signals at the first, second and third detection temperatures can be obtained by using the measured signals when incubating a sample containing only the first target nucleic acid with the first oligonucleotide set. Further, the difference between the signals at the first, second and third detection temperatures can be obtained by using the measured signals when incubating a sample containing only the second target nucleic acid with the second oligonucleotide set. Further, the difference between the signals at the first, second and third detection temperatures can be obtained by using the measured signals when incubating a sample containing only the third target nucleic acid with the third oligonucleotide set. Further, the difference between the signals at the first, second and third detection temperatures can be obtained by using the measured signals when incubating a sample containing the first and second target nucleic acids with the first and second oligonucleotide sets. Further, the difference between the signals at first, second and third detection temperatures can be obtained by using the measured signals when incubating a sample containing the first and third target nucleic acids with the first and third oligonucleotide sets. Further, the difference between the signals at first, second and third detection temperatures can be obtained by using the measured signals when incubating a sample containing the second and third target nucleic acids with the second and third oligonucleotide sets. Further, the difference between the signals at first, second and third detection temperatures can be obtained by using the measured signals when incubating a sample containing the first, second and third target nucleic acids with the first, second and third oligonucleotide sets.

According to an embodiment, the difference between the signals obtained in the above method is a value and the value is used as a reference value with modification or without modification.

According to an embodiment, a reference value may be obtained by calculating the ratio or subtraction. According to an embodiment of this invention, the reference value is obtained by calculating the ratio of two signals measured at two detection temperatures selected from the first detection temperature, the second detection temperature and the third detection temperature.

According to an embodiment, calculation methods for difference of signals from a sample and difference for obtaining a reference value may be the same or different from each other. For instance, the former may be carried out by subtraction of the two signals and the latter by division of the two signals. Alternatively, the former and the latter all may be carried out by division of the two signals to obtain a ratio.

For a target nucleic acid, the reference values may be obtained in various reaction conditions including the amount of component (e.g., the target nucleic acid, signal-generating means, enzymes, or dNTPs), buffer pH or reaction time. According to an embodiment of this invention, the reference value may be obtained under reaction conditions sufficient to provide a saturated signal at the reaction completion. According to an embodiment of this invention, the difference between the signals obtained in calculating the reference value has a certain range and the reference value is selected within the certain range or with referring to the certain range. According to an embodiment of this invention, the reference value may be selected with maximum or minimum value of the certain range or with referring to maximum or minimum value of the certain range. Particularly, the reference value may be modified in considering standard variation of the reference values obtained in various conditions, acceptable error ranges, specificity or sensitivity.

According to an embodiment of this invention, the reference values may be obtained in identical reaction conditions used for the sample including the components (enzymes or amplification primers if used), buffer pH, reaction process. According to an embodiment of this invention, the reference values may be obtained with a signal amplification process concomitantly with or without a nucleic acid amplification.

According to an embodiment of this invention, where there is a significant difference between the reference value and the difference obtained for determining the presence of the first and second target nucleic acids, the first and second target nucleic acids are then determined to be present.

Where there is a significant difference between the reference value reflecting change in signals at the second detection temperature and the third detection temperature for the third target nucleic acid and the difference obtained for determining the presence of the second target nucleic acid, the second target nucleic acid is then determined to be present.

Further, where there is a significant difference between the reference value reflecting change in signals at the second detection temperature and the first detection temperature for the second target nucleic acid, the reference value reflecting change in signals at the third detection temperature and the first detection temperature for the third target nucleic acid and the difference obtained for determining the presence of the first target nucleic acid, the first target nucleic acid is then determined to be present. The reference value may be expressed with the same value type as the difference obtained for determining the presence of the corresponding target nucleic acid (e.g., ratio of end-point values of signal intensities).

Alternatively, the reference value may be used to calculate the difference between two signals selected from signals at the first, second and third detection temperatures. For example, the difference for determining the presence of the first target nucleic acid is calculated such a manner that the signal (e.g., RFU) measured at the third detection temperature is multiplied (or divided) by the reference value reflecting change in signals at the first detection temperature and the third detection temperature for the third target nucleic acid, and the multiplication (or division) result is subtracted from the signal (e.g., RFU) measured at the first detection temperature. In the same manner, the signal measured at the third detection temperature is multiplied (or divided) by the reference value reflecting change in signals at the second detection temperature and the third detection temperature for the third target nucleic acid, and the multiplication (or division) result is subtracted from the signal measured at the second detection temperature, and the result is multiplied (or divided) by the reference value reflecting change in signals at the second detection temperature and the first detection temperature for the second target nucleic acid and the multiplication (or division) result is further subtracted from the signal measured at the first detection temperature. Where a difference is higher (or lower) than “0” or a predetermined value, the first target nucleic acid can be determined to be present.

According to an embodiment, the predetermined value may take a role as a threshold.

According to an embodiment, where the threshold is obtained using the reference value for determining the presence of a certain target nucleic acid, it may be obtained using a certain reference value or some reference values among reference values obtained from various combinations of target nucleic acids. For instance, where it is determined that the third target nucleic acid is not present, a reference value obtained from a combination of target nucleic acids containing not the third target nucleic acid is employed to determine the threshold.

In an embodiment, for verifying the presence of a target nucleic acid having the certain detection temperature, a standard sample comprising combinations of target nucleic acids except for the target nucleic acid having the certain detection temperature is pre-prepared and then reference values are obtained. In considering the reference values, a threshold is obtained for determining the presence of the target nucleic acid having the certain detection temperature.

According to an embodiment, the reference value is used to determine the presence of the first and second target nucleic acids, when a significant signal for the third target nucleic acid is detected or when a difference between the signals at each of the detection temperatures is obtained by mathematical process.

According to an embodiment, where signals are generated in a real-time manner associated with target amplification by PCR, the mathematical processing of the signals comprises calculations of the ratio of signal intensities measured at two detection temperatures at each amplification cycle. The calculation results are plotted against cycles and used for determination of the presence of the first target nucleic acid and the second target nucleic acid.

According to an embodiment, where signals are generated in a real-time manner associated with target amplification by PCR, Ct value is a signal for target detection.

The Ct value of the first target nucleic acid generated by the first oligonucleotide set may be determined using the signals measured at the first, second and third detection temperatures, which is exemplified as follows: Firstly, a real-time PCR is performed for a sample to be analyzed and the signals measured at each of the detection temperatures are obtained, followed by obtaining amplification curves of the three detection temperatures.

For example, in the detection at the third detection temperature, where there is no Ct value of the third target nucleic acid, it can be determined that the third target nucleic acid is not present. Then, the Ct value of the second target nucleic acid is calculated from the amplification curve obtained at the second detection temperature. Where the second target nucleic acid is also absent, there is no Ct value of the second target nucleic acid. Then, the Ct value of the first target nucleic acid is calculated from the amplification curve obtained at the first detection temperature. Where the first target nucleic acid is also absent, there is no Ct value of the first target nucleic acid. The order of detection temperature may vary depending on the signal generation method.

Meanwhile, where there is Ct value of the third target nucleic acid, a ratio of the RFU value obtained at the first detection temperature to the RFU value obtained at the third detection temperature as well as the RFU value obtained at the second detection temperature to the RFU value obtained at the third detection temperature at the cycle showing the Ct value are then calculated. Ratios of RFU values obtained at cycles following the cycle showing the Ct value are also calculated. For example, where all ratios of the RFU values are lower than a reference value (e.g., a value obtained using only the third target nucleic acid as described above), the first target nucleic acid and the second target nucleic acid are determined to be absent. Therefore, there are no Ct values of the first and second target nucleic acids.

Where ratios calculated are the same as the reference value, the determination may be arbitrarily made. For example, the examples described above describe that the determination is made with considering whether the ratios are less than or no less than reference values. In addition, the determination may be made with considering whether the ratios are no more than or more than reference values.

The Ct values of the first and second target nucleic acids may be alternatively calculated as follows: the ratio between two of the RFU values obtained at the first detection temperature, the second detection temperature and the third detection temperature at each cycle is calculated; and Ct value is then calculated with consideration of a threshold value.

The Ct value of the second target nucleic acid may be alternatively calculated as follows: the RFU value obtained at the third detection temperature at each cycle is modified with a reference value of each cycle; the ratio of the RFU value obtained at the second detection temperature to the modified RFU value is calculated for each cycle; and Ct value is then calculated. When calculating the Ct value of the first target nucleic acid in this way, it is necessary to modify the RFU value obtained at the third detection temperature and calculate the ratio of the RFU values obtained at the first and second detection temperatures, considering both the RFU value obtained at the third detection temperature and the RFU value obtained at the second detection temperature.

According to an embodiment of this invention, using the signals detected at the first, second and third detection temperature and the differences comprises obtaining a qualifying value for determining the presence of each of the target nucleic acid.

According to an embodiment of this invention, using the difference comprises obtaining a qualifying value for determining the presence of the first target nucleic acid and/or the second target nucleic acid, and the qualifying value is obtained by (i) mathematically processing the signals measured at the first, second and third detection temperatures or (ii) using the signals per se measured at the first, second and third detection temperatures.

The qualifying values may be further mathematically processed to obtain modified values. The qualifying values are used to determine the presence of the target nucleic acids in the sample.

According to an embodiment of this invention, the step (c) can use at least one reference values for determining the presence of at least one target nucleic acid. For example, the at least one reference value can be used for determining the presence of the first target nucleic acid and/or the second target nucleic acid.

As mentioned above, the reference value may be a value calculated in various ways. For example, the reference value can be any one of the values that reflect change in signals at the first detection temperature and the second detection temperature, generated by the first oligonucleotide set in the presence of the first target nucleic acid, change in signals at the first detection temperature and the third detection temperature, generated by the first oligonucleotide set in the presence of the first target nucleic acid, change in signals at the second detection temperature and the third detection temperature, generated by the first oligonucleotide set in the presence of the first target nucleic acid, change in signals at the first detection temperature and the second detection temperature, generated by the second oligonucleotide set in the presence of the second target nucleic acid, change in signals at the first detection temperature and the third detection temperature, generated by the second oligonucleotide set in the presence of the second target nucleic acid, change in signals at the second detection temperature and the third detection temperature, generated by the second oligonucleotide set in the presence of the second target nucleic acid, change in signals at the first detection temperature and the second detection temperature, generated by the third oligonucleotide set in the presence of the third target nucleic acid, change in signals at the first detection temperature and the third detection temperature, generated by the third oligonucleotide set in the presence of the third target nucleic acid, and change in signals at the second detection temperature and the third detection temperature, generated by the third oligonucleotide set in the presence of the third target nucleic acid. Some of the reference values can be selected, depending upon the method of determining the presence of the target nucleic acid.

The feature of the present invention is to employ three reference values: the reference value reflecting change in signals at the second detection temperature and the third detection temperature, generated by the third oligonucleotide set in the presence of the third target nucleic acid, the reference value reflecting change in signals at the first detection temperature and the third detection temperature, generated by the third oligonucleotide set in the presence of the third target nucleic acid, and the reference value reflecting change in signals at the first detection temperature and the second detection temperature, generated by the second oligonucleotide set in the presence of the second target nucleic acid.

The present inventors have made extensive research to develop an optimal method to determine the presence of multiple target nucleic acids simultaneously based on the signals measured at each of the detection temperatures. Specifically, we have found that by using the above three reference values to extract a signal for each of the target nucleic acids, the presence of each target nucleic acid can be determined in an effective and simple manner, while minimizing false positive/negative errors.

According to an embodiment, the reference value and the measured signals may be used for providing an extracted signal to determine the presence of at least one target nucleic acid.

In certain embodiments, the presence of the first target nucleic acid may be determined from the extracted signal for the first target nucleic acid at the first detection temperature, and the presence of the second target nucleic acid may be determined from the extracted signal for the second target nucleic acid at the second detection temperature.

As mentioned above, the presence of the third target nucleic acid can be determined by assessing the significance of the signal measured at the third detection temperature containing only the signal for the third target nucleic acid. The case in which the third target nucleic acid is absent includes a case in which a signal with similar intensity to a background signal is only detected.

On the other hand, the signal for the first target nucleic acid may be mixed with the signal for the second target nucleic acid and the signal for the third target nucleic acid at the first detection temperature, and the signal for the second target nucleic acid may be mixed with the signal for the third target nucleic acid at the second detection temperature. Therefore, to determine the presence of each target nucleic acid, it is necessary to utilize the differences and the reference values as mentioned above to extract a signal for each of the first target nucleic acid and the second target nucleic acid.

Since the first oligonucleotide set provides a signal only at the first detection temperature, the signal for the first target nucleic acid can be extracted only from the signal measured at the first detection temperature. However, since the second oligonucleotide set provides a signal not only at the second detection temperature but also at the first detection temperature, the signal for the second target nucleic acid can be extracted from either the signal measured at the first detection temperature or the signal measured at the second detection temperature. In this regard, the present inventors have found that the determining the presence of second target nucleic acid using the extracted signal for the second target nucleic acid at the second detection temperature rather than using the extracted signal for the first target nucleic acid at the first detection temperature can lead to more reliable results by minimizing errors in the process of obtaining the reference values and mathematical processing through the various methods described above.

In an embodiment, the extracted signal for the second target nucleic acid at the second detection temperature may be provided by using the reference value reflecting change in signals at the second detection temperature and the third detection temperature, generated by the third oligonucleotide set in the presence of the third target nucleic acid.

For example, the extracted signal for the second target nucleic acid at the second detection temperature may be provided by eliminating a signal for the third target nucleic acid at the second detection temperature from the signal measured at the second detection temperature, wherein the signal for the third target nucleic acid at the second detection temperature is obtained by processing the signal measured at the third detection temperature with the reference value reflecting change in signals at the second detection temperature and the third detection temperature for the third target nucleic acid. In other words, the signal for the third target nucleic acid at the second detection temperature may be obtained by processing the signal measured at the third detection temperature with the reference value reflecting change in signals at the second detection temperature and the third detection temperature for the third target nucleic acid.

In an embodiment, the extracted signal for the second target nucleic acid at the second detection temperature (eS_{T2}) can be provided by processing the following formula:

eS_{T2}=RFU_{D2}−RV_{T3(D2D3)}*RFU_{D3} <Formula I>
eS_{T2}: the extracted signal for the second target nucleic acid at the second detection temperature;
RFU_{D2}: the signal measured at the second detection temperature;
RV_{T3(D2D3)}: the reference value reflecting change in signals at the second detection temperature and the third detection temperature for the third target nucleic acid;
RFU_{D3}: the signal measured at the third detection temperature.

The term used herein “processing a signal with a reference value” refers to the mathematically processing of the measured signal with the reference value, which can be applied to the extraction of signals using various formulas involving multiplication or division to confirm the presence of each target within the same reaction.

The extracted signal for the first target nucleic acid at the first detection temperature may be provided by eliminating from the signal measured at the first detection temperature (i) a signal for the third target nucleic acid at the first detection temperature and (ii) a signal for the second target nucleic acid at the first detection temperature.

In an embodiment, the extracted signal for the first target nucleic acid at the first detection temperature (eS_{T1}) can be provided by the following formula II:

eS_{T1}=RFU_{D1}−eS_{T2}−eS_{T3} <Formula II>
eS_{T1}: the extracted signal for the first target nucleic acid at the first detection temperature;
RFU_{D1}: the signal measured at the first detection temperature;
eS_{T2}: the extracted signal for the second target nucleic acid at the first detection temperature;
eS_{T3}: the extracted signal for the third target nucleic acid at the first detection temperature.

In an embodiment, the extracted signal for the first target nucleic acid at the first detection temperature may be provided by using three reference values: the reference value reflecting change in signals at the second detection temperature and the third detection temperature, generated by the third oligonucleotide set in the presence of the third target nucleic acid, the reference value reflecting change in signals at the first detection temperature and the third detection temperature, generated by the third oligonucleotide set in the presence of the third target nucleic acid, and the reference value reflecting change in signals at the first detection temperature and the second detection temperature, generated by the second oligonucleotide set in the presence of the second target nucleic acid.

In an embodiment, to eliminate an signal for the third target nucleic acid at the first detection temperature from the signal measured at the first detection temperature, the reference value reflecting change in signals at the first detection temperature and the third detection temperature, generated by the third oligonucleotide set in the presence of the third target nucleic acid can be used, whereby the signal for the third target nucleic acid at the first detection temperature is obtained. For example, the signal for the third target nucleic acid at the first detection temperature can be obtained by multiplying the signal measured at the third detection temperature by the reference value reflecting change in signals at the first detection temperature and the third detection temperature for the third target nucleic acid.

On the other hand, to eliminate the signal generated by the third target nucleic acid from the measured signal measured at the first detection temperature, the signal generated by the third target nucleic acid at the first detection temperature can be removed, rather than at the third detection temperature. For example, the signal generated by the third target nucleic acid at the first detection temperature can be obtained by i) multiplying the signal measured at the third detection temperature by the reference value reflecting change in signals at the second detection temperature and the third detection temperature for the third target nucleic acid to obtain a signal generated by the third target nucleic acid at the second detection temperature, ii) further multiplying the signal generated by the third target nucleic acid at the second detection temperature obtained in i) by the reference value reflecting change in signals at the first detection temperature and the second detection temperature for the third target nucleic acid. This may cause errors during the delicate calibration or plotting process of signals and reference values, when configuring the signal indicating the presence of the target nucleic acid by performing the present method on a program. Therefore, the present invention determines the presence of the first target nucleic acid without using the reference value reflecting change in signals at the first detection temperature and the second detection temperature for the third target nucleic acid.

In an embodiment, to remove the signal generated by the second target nucleic acid from the signal measured at the first detection temperature, first, it is necessary to extract the signal generated by the second target nucleic acid at the second detection temperature. This may be the same as the method mentioned above for extracting a signal to determine the presence of the second target nucleic acid. For example, the extracted signal for the second target nucleic acid at the second detection temperature can be provided by eliminating a signal for the third target nucleic acid at the second detection temperature from the signal measured at the second detection temperature, wherein the signal for the third target nucleic acid at the second detection temperature is obtained by processing the signal measured at the third detection temperature with the reference value reflecting change in signals at the second detection temperature and the third detection temperature for the third target nucleic acid. Then, the signal for the second target nucleic acid at the first detection temperature can be obtained using the extracted signal for the second target nucleic acid at the second detection temperature and the reference values reflecting change in signals at the first and second detection temperatures for the second target nucleic acid.

In an embodiment, the extracted signal for the first target nucleic acid at the first detection temperature (eST1) can be provided by the following formula III:

eS_{T1}=RFU_{D1}−(RFU_{D2}−RV_{T3(D2D3})*RFU_{D3})−RV_{T3(D1D3)}*RFU_{D3} <Formula III>
eS_{T1}: the extracted signal for the first target nucleic acid at the first detection temperature;
RFU_{D2}: the signal measured at the second detection temperature;
RV_{T3(D2D3)}: the reference value reflecting change in signals at the second detection temperature and the third detection temperature for the third target nucleic acid;
RFU_{D3}: the signal measured at the third detection temperature;
RV_{T3(D1D3)}: the reference value reflecting change in signals at the first detection temperature and the third detection temperature for the third target nucleic acid;
RFU_{D3}: the signal measured at the third detection temperature.

In certain embodiments, when it is determined that the third target nucleic acid is absent, the presence of the second target nucleic acid can be determined by the signal measured at the second detection temperature. When the third target nucleic acid is absent, the second target nucleic acid only provides the significant signal at the second detection temperature. Therefore, the presence of the second target nucleic acid can be determined by considering the significance of the signal measured at the second detection temperature.

In certain embodiments, wherein when it is determined that the second target nucleic acid is absent, the extracted signal for the first target nucleic acid at the first detection temperature is provided by eliminating an signal for the third target nucleic acid at the first detection temperature from the signal measured at the first detection temperature, wherein the signal for the third target nucleic acid at the first detection temperature is obtained by processing the signal measured at the third detection temperature with the reference value reflecting change in signals at the first detection temperature and the third detection temperature for the third target nucleic acid.

### II. Kits for Detection of Target Nucleic Acids

In another aspect of this invention, there is provided a kit for detecting target nucleic acids of at least nine human papillomavirus (HPV) types in a sample, comprising:
(a) at least nine oligonucleotide sets to amplify or detect target nucleic acids of at least nine HPV types,
wherein each of the at least nine oligonucleotide sets comprises:
(i) an amplifying oligonucleotide, which serves to amplify a target nucleic acid of an HPV type of interest; and
(ii) a signaling oligonucleotide having a fluorescent label linked thereto, which serves to generate a signal in the presence of a target nucleic acid of an HPV type of interest,
wherein the amplifying oligonucleotide and the signaling oligonucleotide are identical to or different from each other,
wherein the fluorescent labels included in the at least nine oligonucleotide sets are of at least three different types, which are distinguishable by at least three different detection channels, and three of the at least nine oligonucleotide sets comprise any one of the at least three different types of the fluorescent labels,
wherein for three oligonucleotide sets comprising any one of the at least three different types of the fluorescent labels, one oligonucleotide set is designed to generate a signal at a first detection temperature in the presence of a target nucleic acid of a corresponding HPV type, another oligonucleotide set is designed to generate a signal at the first detection temperature and a second detection temperature in the presence of a target nucleic acid of another corresponding HPV type, and the other oligonucleotide set is designed to generate a signal at the first detection temperature, the second detection temperature and a third detection temperature in the presence of a target nucleic acid of the other corresponding HPV type;
(b) an instruction that describes the method of the Aspect I.

Since the kits of this invention are prepared to perform the present methods, the common descriptions between them are omitted in order to avoid undue redundancy leading to the complexity of this specification.

All of the present kits described hereinabove may optionally include the reagents required for performing target amplification reactions (e.g., PCR reactions) such as buffers, DNA polymerase cofactors, and deoxyribonucleotide-5-triphosphates. Optionally, the kits may also include various polynucleotide molecules, reverse transcriptase, various buffers and reagents, and antibodies that inhibit DNA polymerase activity. The kits may also include reagents necessary for performing positive and negative control reactions. Optimal amounts of reagents to be used in a given reaction can be readily determined by the skilled artisan having the benefit of the current disclosure. The components of the kit may be present in separate containers, or multiple components may be present in a single container.

The instructions for describing or practicing the methods of the present invention may be recorded on a suitable recording medium. For example, the instructions may be printed on a substrate, such as paper and plastic. In other embodiments, the instructions may be present as an electronic storage data file present on a suitable computer readable storage medium such as CD-ROM and diskette. In yet other embodiments, the actual instructions may not be present in the kit, but means for obtaining the instructions from a remote source, e.g., via the internet, are provided. An example of this embodiment is a kit that includes a web address where the instructions can be viewed and/or from which the instructions can be downloaded.

### III. Storage Medium and Device for Detection of Target Nucleic Acids

Since the storage medium, the device and the computer program of the prevent invention described hereinafter are intended to perform the present methods in a computer, the common descriptions between them are omitted in order to avoid undue redundancy leading to the complexity of this specification.

In another aspect of this invention, there is provided a computer readable storage medium containing instructions to configure a processor to perform a method of detecting target nucleic acids of at least nine human papillomavirus (HPV) types in a sample, the method comprising:
(a) receiving signals measured at a first detection temperature, a second detection temperature, and a third detection temperature using each of at least three detection channels,
wherein the signals are obtained by incubating the sample in a single reaction vessel with at least nine oligonucleotide sets to amplify or detect target nucleic acids of at least nine HPV types and measuring signals at the first detection temperature, the second detection temperature, and the third detection temperature using each of the at least three detection channels,
wherein each of the at least nine oligonucleotide sets comprises:
(i) an amplifying oligonucleotide, which serves to amplify a target nucleic acid of an HPV type of interest; and
(ii) a signaling oligonucleotide having a fluorescent label linked thereto, which serves to generate a signal in the presence of a target nucleic acid of an HPV type of interest,
wherein the amplifying oligonucleotide and the signaling oligonucleotide are identical to or different from each other,
wherein the fluorescent labels included in the at least nine oligonucleotide sets are of at least three different types, which are distinguishable by at least three different detection channels, and three of the at least nine oligonucleotide sets comprise any one of the at least three different types of the fluorescent labels,
wherein for three oligonucleotide sets comprising any one of the at least three different types of the fluorescent labels, one oligonucleotide set is designed to generate a signal at a first detection temperature in the presence of a target nucleic acid of a corresponding HPV type, another oligonucleotide set is designed to generate a signal at the first detection temperature and a second detection temperature in the presence of a target nucleic acid of another corresponding HPV type, and the other oligonucleotide set is designed to generate a signal at the first detection temperature, the second detection temperature and a third detection temperature in the presence of a target nucleic acid of the other corresponding HPV type;
(b) determining the presence of target nucleic acids of the at least nine HPV types by signals measured at the first detection temperature, the second detection temperature, and the third detection temperature using each of the at least three detection channels.

According to an embodiment, particular reference values are used to determine the presence of target nucleic acids of at least nine HPV types.

According to an embodiment of this invention, the reference values are stored in the computer readable storage medium. According to an embodiment of the present invention, the computer readable storage medium contains instructions to input the reference values in performing the method. According to an embodiment of the present invention, the computer readable storage medium further contains instructions to configure a processor to perform a method for obtaining the reference values.

In another aspect of this invention, there is provided a computer program to be stored on a computer readable storage medium to configure a processor to perform a method of detecting target nucleic acids of at least nine human papillomavirus (HPV) types in a sample, the method comprising:
(a) receiving signals measured at a first detection temperature, a second detection temperature, and a third detection temperature using each of at least three detection channels,
wherein the signals are obtained by incubating the sample in a single reaction vessel with at least nine oligonucleotide sets that are designed to amplify or detect target nucleic acids of at least nine HPV types and measuring signals at the first detection temperature, the second detection temperature, and the third detection temperature using each of the at least three detection channels,
wherein each of the at least nine oligonucleotide sets comprises:
(i) an amplifying oligonucleotide, which serves to amplify a target nucleic acid of an HPV type of interest; and
(ii) a signaling oligonucleotide having a fluorescent label linked thereto, which serves to generate a signal in the presence of a target nucleic acid of an HPV type of interest,
wherein the amplifying oligonucleotide and the signaling oligonucleotide are identical to or different from each other,
wherein the fluorescent labels included in the at least nine oligonucleotide sets are of at least three different types, which are distinguishable by at least three different detection channels, and three of the at least nine oligonucleotide sets comprise any one of the at least three different types of the fluorescent labels,
wherein for three oligonucleotide sets comprising any one of the at least three different types of the fluorescent labels, one oligonucleotide set is designed to generate a signal at a first detection temperature in the presence of a target nucleic acid of a corresponding HPV type, another oligonucleotide set is designed to generate a signal at the first detection temperature and a second detection temperature in the presence of a target nucleic acid of another corresponding HPV type, and the other oligonucleotide set is designed to generate a signal at the first detection temperature, the second detection temperature and a third detection temperature in the presence of a target nucleic acid of the other corresponding HPV type;
(b) determining the presence of target nucleic acids of the at least nine HPV types by signals measured at the first detection temperature, the second detection temperature, and the third detection temperature using each of the at least three detection channels.

The program instructions are operative, when performed by the processor, to cause the processor to perform the present method described above. The program instructions may comprise an instruction to receive the first signal and the second signal, and an instruction to determine the presence of at least nine target nucleic acids by using the signals received.

The present method described above is implemented in a processor, such as a processor in a stand-alone computer, a network attached computer or a data acquisition device such as a real-time PCR machine.

The types of the computer readable storage medium include various storage medium such as CD-R, CD-ROM, DVD, flash memory, floppy disk, hard drive, portable HDD, USB, magnetic tape, MINIDISC, nonvolatile memory card, EEPROM, optical disk, optical storage medium, RAM, ROM, system memory and web server.

The data (e.g., intensity, amplification cycle number and detection temperature) associated with the signals may be received through several mechanisms. For example, the data may be acquired by a processor resident in a PCR data acquiring device. The data may be provided to the processor in real time as the data is being collected, or it may be stored in a memory unit or buffer and provided to the processor after the experiment has been completed. Similarly, the data set may be provided to a separate system such as a desktop computer system via a network connection (e.g., LAN, VPN, intranet and Internet) or direct connection (e.g., USB or other direct wired or wireless connection) to the acquiring device, or provided on a portable medium such as a CD, DVD, floppy disk, portable HDD or the like to a standalone computer system. Similarly, the data set may be provided to a server system via a network connection (e.g., LAN, VPN, intranet, Internet and wireless communication network) to a client such as a notebook or a desktop computer system. After the data has been received or acquired, the data analysis process proceeds to give a processed signal obtained from a difference between the signals for determination of the presence of target nucleic acids when the signal is detected at each of the detection temperatures. The processor processes the received data associated with the signals to give the processed signal reflecting the difference between two signals selected from signals measured at the three detection temperatures.

The instructions to configure the processor to perform the present invention may be included in a logic system. The instructions may be downloaded and stored in a memory module (e.g., hard drive or other memory such as a local or attached RAM or ROM), although the instructions can be provided on any software storage medium such as a portable HDD, USB, floppy disk, CD and DVD. A computer code for implementing the present invention may be implemented in a variety of coding languages such as C, C++, Java, Visual Basic, VBScript, JavaScript, Perl and XML. In addition, a variety of languages and protocols may be used in external and internal storage and transmission of data and commands according to the present invention.

In still another aspect of this invention, there is provided a device for detecting target nucleic acids of at least nine human papillomavirus (HPV) types in a sample, comprising (a) a computer processor and (b) the computer readable storage medium described above coupled to the computer processor.

According to an embodiment, the device further comprises a reaction vessel to accommodate the sample and oligonucleotide sets, a temperature controlling means to control temperatures of the reaction vessel and/or a single type of detector to detect signals to be generated by the oligonucleotide sets.

According to an embodiment, the processor may be embodied by installing software into conventional devices for detection of target nucleic acids (e.g., real-time PCR device). According to an embodiment, the device comprises a processor to permit the device to detect signals and to extract signal for each target nucleic acid.

The present invention will now be described in further detail by examples. It would be obvious to those skilled in the art that these examples are intended to be more concretely illustrative and the scope of the present invention as set forth in the appended claims is not limited to or by the examples.

### MODE FOR THE INVENTION

### EXAMPLES

### Example 1: Determination of the Presence of 14 HPV Types and Internal Control Using Signals Measured at Three Detection Temperatures

It was investigated whether 15 target nucleic acids including 14 HPV types and one internal control could be detected in a single reaction vessel by PTOCE real-time PCR for measuring signals at three different temperatures under five detection channels.

### <1-1> Amplification and Signal Detection of 14 HPV Types and Internal Control

Taq DNA polymerase having 5′ nuclease activity was used for the extension of forward primers and reverse primers, the cleavage of PTO, and the extension of PTO fragment.

Genomic DNAs of HPV 16, 18, 31, 33, 35, 39, 45, 51, 52, 56, 58, 59, 66, 68 and internal control were used as target nucleic acids.

PTOCE real-time PCR was used to detect target nucleic acids.

In this Example, five detection channels were used to detect five types of fluorescent signals. As fluorescent labels for generating the fluorescent signals, FAM (Biosearch Technologies Inc), CAL Fluor Orange 560 (Biosearch Technologies Inc), CAL Fluor Red 610 (Biosearch Technologies Inc), ATTO 647N (ATTO-TEC GmbH), and ATTO (ATTO-TEC GmbH) were used.

Detection temperatures for each channel were selected as 60° C., 72° C. and 83° C. The HPV types assigned to each detection temperature for each channel are shown in Table 1.

TABLE 1

| | 60° C. | 72° C. | 83° C. |
|---|---|---|---|
| FAM | HPV 66 | HPV 45 | HPV 58 |
| CAL Fluor Orange 560 | HPV 51 | HPV 59 | HPV 16 |
| CAL Fluor Red 610 | HPV 33 | HPV 39 | HPV 52 |
| ATTO 647N | Internal Control | HPV 35 | HPV 18 |
| ATTO 680 | HPV 56 | HPV 68 | HPV 31 |

As shown in table 1, for the target nucleic acids assigned to the detection temperature of 60° C., the CTOs (Capturing and Templating Oligonucleotides) used in the PTOCE method were designed to form a duplex at 60° C. to provide a signal, but not to form a duplex at 72° C. and 83° C. to provide no signal; for the target nucleic acids assigned to the detection temperature of 72° C., the CTOs used in the PTOCE method were designed to form a duplex at 60° C. and 72° C. to provide a signal, but not to form a duplex at 83° C. to provide no signal; for the target nucleic acids assigned to the detection temperature of 83° C., the CTOs used in the PTOCE method were designed to form a duplex at 60° C., 72° C. and 83° C. to provide a signal.

Therefore, at 83° C., only the signal of the target nucleic acid assigned to the detection temperature of 83° C. can be generated and detected; at 72° C., not only the signal of the target nucleic acid assigned to the detection temperature of 72° C., but also the signal of the target nucleic acid assigned to the detection temperature of 83° C. can be generated and detected in a mixed form that cannot be distinguished from each other; and at 60° C., the signal of the target nucleic acid assigned to the detection temperature of 60° C., the signal of the target nucleic acid assigned to the detection temperature of 72° C., and the signal of the target nucleic acid assigned to the detection temperature of 83° C. can be generated and detected in a mixed form that cannot be distinguished from each other.

The PTOs and CTOs were blocked with a carbon spacer at their 3′-ends to prohibit their extension. The CTOs were each labeled with a quencher molecule (BHQ-2) and a fluorescent reporter molecule (one of FAM, CAL Fluor Orange 560, CAL Fluor Red 610, ATTO 647N and ATTO 680) in its templating portion.

One reaction tube was prepared containing HPV 16, 18, 31, 33, 35, 39, 45, 51, 52, 56, 58, 59, 66, 68 and an internal control.

The sequences of forward primers, reverse primers, PTOs, and CTOs used in this Example are shown in Table 2:

TABLE 2

| Name | Type | Sequence (5′-> 3′) | SEQ ID |
|---|---|---|---|
| HPV16 | Forward | TTTTTTCAGATGTCTCTTTGGCTGCCTAG | 1 |
| F | primer | | |
| HPV16 | Reverse | ATAGGAAAATAGGGATGTCCAACTGCAAG | 2 |
| R | primer | | |
| HPV16 | PTO | CACCACAGGGTTGAATATGTTGCACGCACA | 3 |
| P | | AACA | |
| HPV16 | CTO | TTTTTTTTTATAATATTTACTAATGGAACCCT | \|4 |
| C | | GTGGTG | |
| HPV18 | Forward | GGGTTACATTACCATCTACTACCTCTGTATG | 5 |
| F | primer | G | |
| HPV18 | Reverse | CTTGTGCGAGTCACATAATCATCGGTATTTA | 6 |
| R | primer | | |
| HPV18 | PTO | ACAGCCTAGCGTTGGCTTTGTGGCGGCCTAG | 7 |
| P | | TGACAA | |
| HPV18 | CTO | ATTATTTTTATTATTTTTTTTATTCCACGCTA | 8 |
| C | | GGCTGT | |
| HPV31 | Forward | TATTATCACGCAGGCAGTGIIIIICTGCTTACA | 9 |
| F | primer | G | |
| HPV31 | Reverse | CCACCGGCATATCTATTAGAGTIIIIIGTGTCA | 10 |
| R | primer | TCA | |
| HPV31 | PTO | CACCAAAGTCGGCCTAAAAAAATAGTTGTA | 11 |
| P | | CCAAAGGTGTCAGGAT | |
| HPV31 | CTO | ATTTTTTTTTTTTTATATTATAATGGCCGACT | 12 |
| C | | TTGGTG | |
| HPV33 | Forward | TGAGGCCACAGTGTACCTGCCTC | 13 |
| F | primer | | |
| HPV33 | Reverse | TTTTTAATAGAAAAATATGGATGGCCAAC | 14 |
| R | primer | | |
| HPV33 | PTO | CTCCATGGTGCATGTGTCTCGCACAAGCATT | 15 |
| P | | TATTAT | |
| HPV33 | CTO | TATTATTTTTATTTATTTTATCTGTCTGCACC | 16 |
| C | | ATGGAG | |
| HPV35 | Forward | TAAATATGTTGGTAACTCTGGTACIIIIIACAG | 17 |
| F | primer | GGAATG | |
| HPV35 | Reverse | CTTGTAGTACAGTGTTTAGTAACIIIIIAGGAG | 18 |
| R | primer | GACAT | |
| HPV35 | PTO | CCAATCGGGACTGGGAAAAGGCACACCTTG | 19 |
| P | | TAATGCT | |
| HPV35 | CTO | ATTATTTTTTATTTATTTAGTATGTCAGTCCC | 20 |
| C | | GATTGG | |
| HPV39 | Forward | TGTCTGCATATCAATATAGGGTATTIIIIITGA | 21 |
| F | primer | CATTGC | |
| HPV39 | Reverse | CACATTATCCCTACTGTCCTIIIIIGTGGTTGAT | 22 |
| R | primer | G | |
| HPV39 | PTO | AAGCTGACACCCGTGTAGGGGTGGAGGTGG | 23 |
| P | | GCAGG | |
| HPV39 | CTO | ATTTTTTTTATTATTATTTATATCCTGGGTGT | 24 |
| C | | CAGCTT | |
| HPV45 | Forward | ARTGTGTCAGTTGATYATAAGCIIIIICAGCTG | 25 |
| F | primer | WGT | |
| HPV45 | Reverse | CAATAATGGTGTTTTTAAGTTCCAAAIIIIIAC | 26 |
| R | primer | ARTCACCA | |
| HPV45 | PTO | AAATGWCCGTCRCAGGTTTACAAAGTGTGC | 27 |
| P | | CCTTGG | |
| HPV45 | CTO | TTTTTTTTTACACTGGCTACTCTCTGGGACG | 28 |
| C | | GGCATTT | |
| HPV51 | Forward | ACACAGAAAATTCACGCATIIIIIATGGCAATG | 29 |
| F | primer | C | |
| HPV51 | Reverse | AACCCTGTATCAATCATATCGIIIIICTGAATG | 30 |
| R | primer | AC | |
| HPV51 | PTO | GGAACACTCCGTGGGTATTGCCACTACATGC | 31 |
| P | | AAAAAC | |
| HPV51 | CTO | TTTTTTTTTTTATTTAATATTAATGGACGGAG | 32 |
| C | | TGTTCC | |
| HPV52 | Forward | ATGATACTGAAACCAGTAACAAATIIIIIGGTA | \|33 |
| F | primer | ARCCT | |
| HPV52 | Reverse | TGTATTACACTGTTAATGAGYTGTIIIIIAGGA | 34 |
| R | primer | CAATCC | |
| HPV52 | PTO | AAGCAAGGACGCTGCATTTTAGGATGCAAA | 35 |
| P | | CCTCCTATAGGTGAA | |
| HPV52 | CTO | ATTATTTTTTTTATTATTTATATCCTGCGTCC | 36 |
| C | | TTGCTT | |
| HPV56 | Forward | TAAGGACAATACYAAAACAAACATTIIIIIAGT | 37 |
| F | primer | TAGTGCA | |
| HPV56 | Reverse | TTTGCTAAATTGGAACTTTCAGTIIIIICCAGCC | 38 |
| R | primer | TAT | |
| HPV56 | PTO | CCACAAGTCTCGCCGGACCAGGAACGGTTA | 39 |
| P | | GTGT | |
| HPV56 | CTO | TTTTTTTTTTTTATAATTATTATAGGCGAGAC | \|40 |
| C | | TTGTGG | |
| HPV58 | Forward | GTTTAATTGGCTGTAAACCTCCIIIIIGTGAGC | 41 |
| F | primer | ATTG | |
| HPV58 | Reverse | ATCAATAGGCACATCACTTTTATTIIIIIGCAA | 42 |
| R | primer | TGTACC | |
| HPV58 | PTO | AACGCCTGTGCTTGCAGCTGCTACTGATTGT | 43 |
| P | | CCTCC | |
| HPV58 | CTO | AGCGGTCTCCCAGTCGTTAGTAGGCAAGCA | 44 |
| C | | CAGGCGTT | |
| HPV59 | Forward | ACAACAAGGTGTATCTACCTIIIIITTCGGTAG | 45 |
| F | primer | CT | |
| HPV59 | Reverse | ACCTTAGGAACATCCTGTCTIIIIITACCACCTT | 46 |
| R | primer | T | |
| HPV59 | PTO | AATCCCTCGGCACTGCGTGGTAGAAAATAC | 47 |
| P | | TGGTACGG | |
| HPV59 | CTO | TTTTTTTTTACAGTGGGTGATTTCAGTGCCG | 48 |
| C | | AGGGATT | |
| HPV66 | Forward | ACTCTGTTTCYAAATCTGGTACIIIIICRAACA | 49 |
| F | primer | TCC | |
| HPV66 | Reverse | AGTGTCATCCAGCCTATTAAATAIIIIITGACC | 50 |
| R | primer | ACTT | |
| HPV66 | PTO | CCAAGCAGAGACCCCATACYAAACGTTCCT | 51 |
| P | | GGTCAGGA | |
| HPV66 | CTO | TTTTTTTTTAATTTTAATTATTATGGGTCTCT | 52 |
| C | | GCTTGG | |
| HPV68 | Forward | ATTAYTATGCTGGTACATCTAGGTIIIIIACTG | 53 |
| F | primer | TAGGCC | |
| HPV68 | Reverse | GGCTGCCCCCTACCTIIIIIAACACCAACA | 54 |
| R | primer | | |
| HPV68 | PTO | GTCCCACCTACACCTGCTTGCGGCYCCCAGA | 55 |
| P | | C | |
| HPV68 | CTO | TTTTTTTTTAACTTTGGTGCTCGAGGTGTAG | 56 |
| C | | GTGGGAC | |
| HBB F | Forward | GCAATAATAGAGAAAGCATTTAAGAGAIIIII | 57 |
| | primer | GCAATGGAAA | |
| HBB R | Reverse | CATCCCCCTGTACTTTTTCCCCTTGIIIIIAATT | 58 |
| | primer | AACT | |
| HBB P | PTO | ACGCTGGGCATTAGAAGTAGGAGAAACATG | 59 |
| | | CAAAGTAAAAGT | |
| HBB C | CTO | ATTTTATTATTTATTATTTTTATTCCAATGCC | 60 |
| | | CAGCGT | |

The real-time PCR was conducted in the final volume of 20 μl containing target nucleic acids (HPV 16, 18, 31, 33, 35, 39, 45, 51, 52, 56, 58, 59, 66, 68 and IC, each pg), and 1˜3.2 pmole of forward primer, 1˜3.2 pmole of reverse primer, 0.8˜2.8 pmole of PTO and 0.2˜0.6 pmole of CTO for each target nucleic acid amplification, and 10 μl of 2× Master Mix [final, 200 μM dNTPs, 2 mM MgCI₂, 2 U of Taq DNA polymerase].

The tube containing the reaction mixture was placed in the real-time thermocycler (CFX96, Bio-Rad), and subjected to denaturation for 15 min at 95° C. and 45 cycles of 3 sec at 95° C., 10 sec at 60° C., 10 sec at 72° C. and 5 sec at 83° C. Detection of signals was performed at 60° C., 72° C. and 83° C. of each cycle.

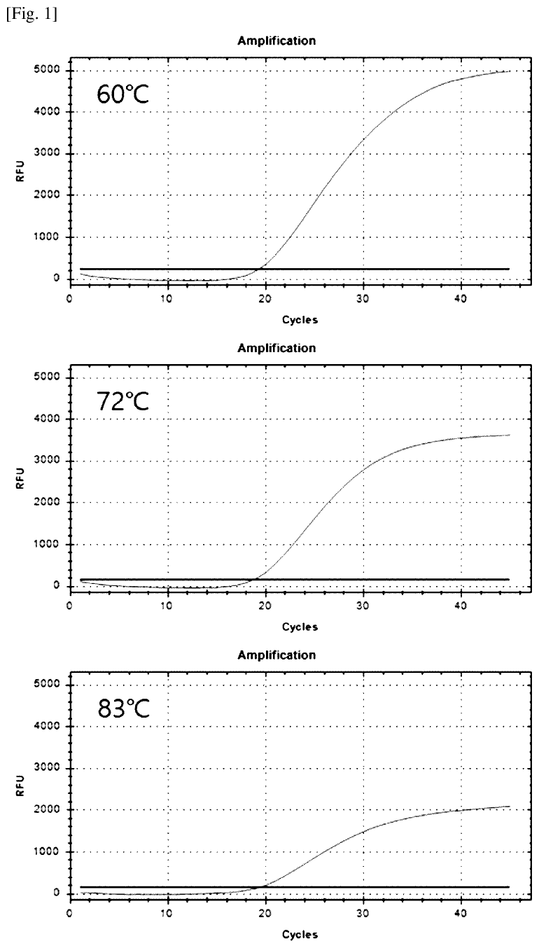

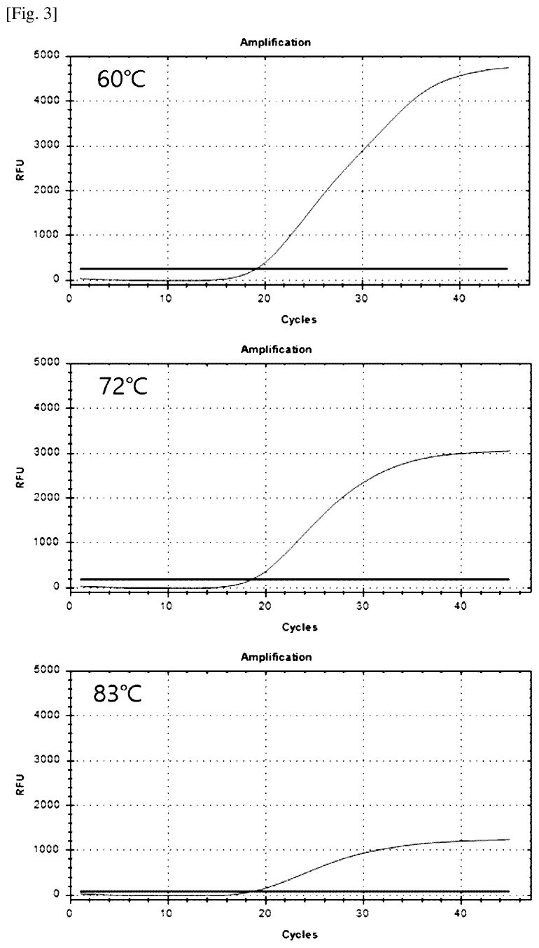

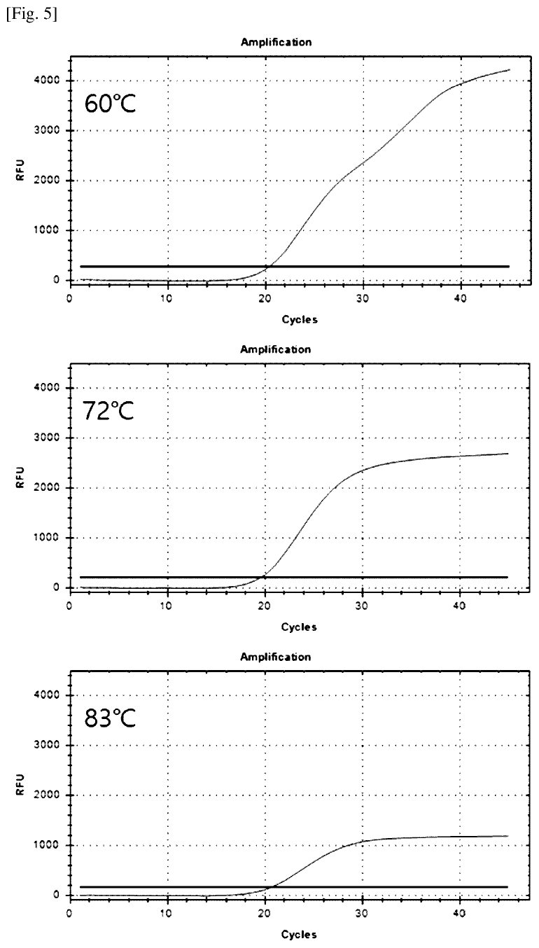

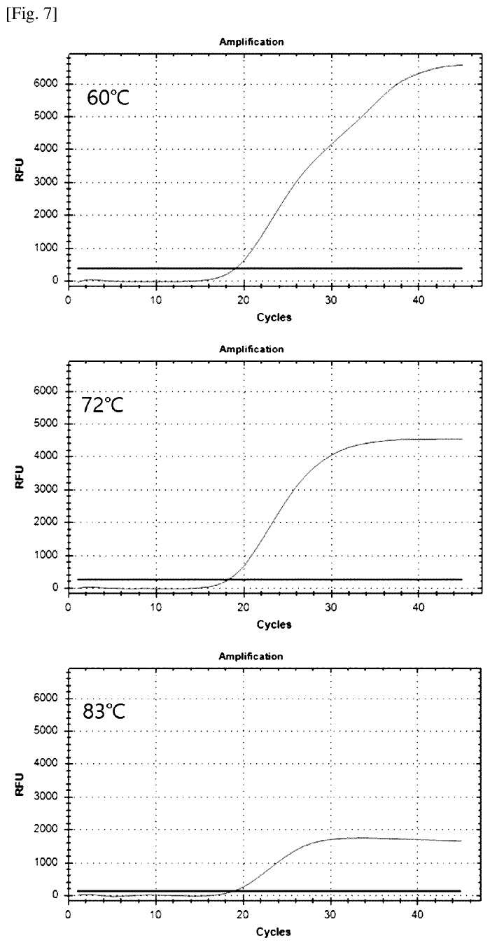

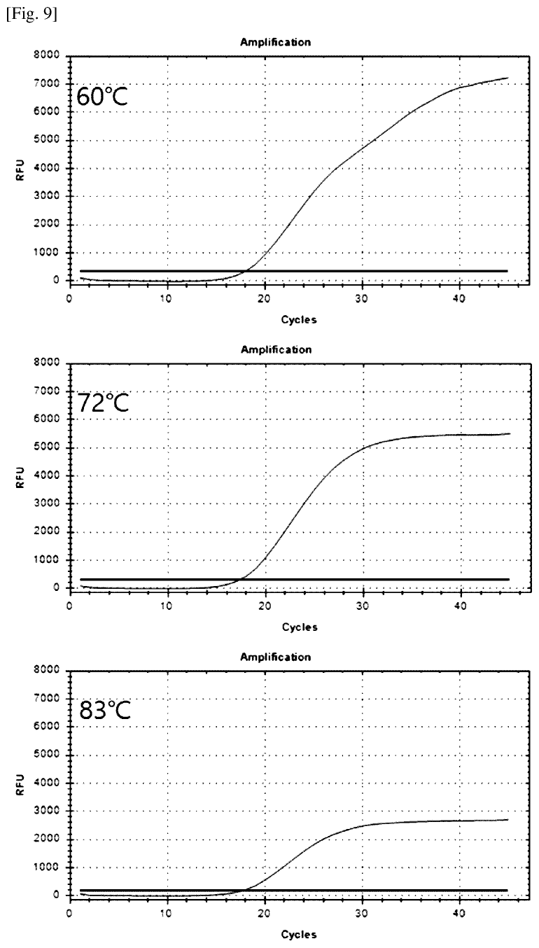

Signals measured at 60° C., 72° C., and 83° C. of each cycle of the PCR reaction for each detection channel are shown in FIGS. 1, 3, 5, 7, and 9.

As shown in FIGS. 1, 3, 5, 7, and 9, the signal measured at the detection temperature of 60° C. for each detection channel included mixed signals for three HPV types and the signal measured at the detection temperature 72° C. for each detection channel included mixed signals for two HPV types. Thus, the presence of each HPV type cannot be determined from the measured signals per se.

### <1-2> Determination of the Presence of 14 HPV Types and Internal Control

In this Example, the reference value for each target nucleic acid and the signals measured at the three detection temperatures (83° C., 72° C. and 60° C.) were used to provide an extracted signal that indicates the presence of each target nucleic acid. Specifically, the presence or absence of the first and second target nucleic acids was determined based on the extracted signal provided by using the reference value and the presence or absence of the third target nucleic acid was determined from the signal measured at the third detection temperature (83° C.) as the signal at 83° C. includes only the signal generated by the third oligonucleotide set.

The reference values of target nucleic acids were determined by analyzing signals from a standard sample containing only one of HPV 16, 18, 31, 35, 39, 45, 52, 58, 59 and 68 and are shown in Tables 3 to 5.

TABLE 3

| | End-RFU | | |
|---|---|---|---|
| | 60° C. | 72° C. | Reference Value |
| HPV 45 | 2026 | 1586 | 1.28 |
| HPV 59 | 1993 | 1652 | 1.21 |
| HPV 39 | 1827 | 1680 | 1.09 |
| HPV 35 | 2268 | 2202 | 1.03 |
| HPV 68 | 2364 | 2341 | 1.01 |

TABLE 4

| | End-RFU | | |
|---|---|---|---|
| | 60° C. | 83° C. | Reference Value |
| HPV 58 | 2149 | 1781 | 1.21 |
| HPV 16 | 1419 | 1420 | 1.00 |
| HPV 52 | 1719 | 1824 | 0.94 |
| HPV 18 | 1702 | 1695 | 1.00 |
| HPV 31 | 2407 | 2426 | 1.00 |

TABLE 5

| | End-RFU | | |
|---|---|---|---|
| | 72° C. | 83° C. | Reference Value |
| HPV 58 | 2064 | 1781 | 1.16 |
| HPV 16 | 1442 | 1420 | 1.02 |
| HPV 52 | 1924 | 1824 | 1.05 |
| HPV 18 | 1775 | 1695 | 1.05 |
| HPV 31 | 2687 | 2426 | 1.11 |

To distinguish the presence of each of three target nucleic acids per each channel, the signals measured at the detection temperatures and the extracted signal for each target obtained from the measured signals were used.

Specifically, the presence of each of HPV 58, HPV 16, HPV 52, HPV 18, and HPV 31, which generate signals at 60° C., 72° C., and 83° C., was determined directly from the signal measured at 83° C. without the signal extraction.

Meanwhile, the presence of each of HPV 45, HPV 59, HPV 39, HPV 35, and HPV 68, which generate signals at 60° C. and 72° C., was determined based on the extracted signal for each channel. The extracted signal was obtained by multiplying the signal measured at 83° C. by a reference value reflecting change in signals at 72° C. and 83° C. for as described in Table 5 (for the target nucleic acid that generates signals at 60° C., 72° C. and 83° C.) and subtracting the multiplied signal from the signal measured at 72° C. The reference value was obtained by calculating a ratio of a signal at 72° C. to a signal at 83° C. for a standard sample containing only one target nucleic acid that generates signals at 60° C., 72° C., and 83° C. Specifically, the extracted signal for HPV 45 at 72° C. was obtained by multiplying the signal measured at 83° C. by the reference value for HPV 58 (1.16) and subtracting the multiplied signal from the signal measured at 72° C., in the FAM channel. The extracted signal for HPV 59 at 72° C. was obtained by multiplying the signal measured at 83° C. by the reference value for HPV 16 (1.02) and subtracting the multiplied signal from the signal measured at 72° C., in the CAL Fluor Orange 560 channel. The extracted signal for HPV 39 at 72° C. was obtained by multiplying the signal measured at 83° C. by the reference value for HPV 52 (1.05) and subtracting the multiplied signal from the signal measured at 72° C., in the CAL Fluor Red channel. The extracted signal for HPV 35 at 72° C. was obtained by multiplying the signal measured at 83° C. by the reference value for HPV 18 (1.05) and subtracting the multiplied signal from the signal measured at 72° C., in the ATTO 647N channel. The extracted signal for HPV 68 at 72° C. was obtained by multiplying the signal measured at 83° C. by the reference value for HPV 31 (1.11) and subtracting the multiplied signal from the signal measured at 72° C., in the ATTO 680 channel.

Further, in case of HPV 66, HPV 51, HPV 33, internal control, and HPV 56 which generate a signal only at 60° C., the presence of each target nucleic acid was determined based on an extracted signal obtained by eliminating signals for the other target nucleic acids from the signal measured at 60° C. by using the signals measured at 72° C. and 83° C. and the reference values, as follows:

First, the extracted signal for the target nucleic acid that generates signals at 60° C. and 72° C. was obtained by multiplying the signal measured at 83° C. by a reference value reflecting change in signals at 72° C. and 83° C. as described in Table 5 (for the target nucleic acid that generates signals at 60° C., 72° C. and 83° C.) and subtracting the multiplied signal from the signal measured at 72° C. The reference value was obtained by calculating a ratio of a signal at 72° C. to a signal at 83° C. for a standard sample containing only one target nucleic acid that generates signals at 60° C., 72° C., and 83° C.

Next, the extracted signal for the target nucleic acid that generates signals at 60° C. and 72° C. was multiplied by a reference value reflecting change in signals at 60° C. and at 72° C. as described in Table 3 (for the target nucleic acid that generates signals at 60° C. and 72° C.) and the multiplied signal was subtracted from the signal measured at 60° C. Then, the signal measured at 83° C. multiplied by a reference value reflecting change in signals at 60° C. and at 83° C. as described in Table 4 (for the target nucleic acid that generate signals at 60° C., 72° C., and 83° C.) was further subtracted from the subtracted signal to provide the extracted signal for determining the presence of each target nucleic acid.

Specifically, the extracted signal for HPV 66 at 60° C. for FAM channel was obtained as follows:

First, the extracted signal for HPV 45 at 72° C. was obtained by multiplying the signal measured at 83° C. by the reference value for HPV 58 (1.16) and subtracting the multiplied signal from the signal measured at 72° C. Second, the extracted signal for HPV 45 at 60° C. was obtained by multiplying the extracted signal for HPV 45 at 72° C. by the reference value for HPV 45 (1.28). Third, the extracted signal for HPV 58 at 60° C. was obtained by multiplying the signal measured at 83° C. by the reference value for HPV 58 (1.21). Fourth, the extracted signal for HPV 45 at 60° C. and the extracted signal for HPV 58 at 60° C. were subtracted from the signal measured at 60° C., thereby obtaining the extracted signal for HPV 66 at 60° C.

The extracted signal for HPV 51 at 60° C. for CAL Fluor Orange 560 channel was obtained as follows:

First, the extracted signal for HPV 59 at 72° C. was obtained by multiplying the signal measured at 83° C. by the reference value for HPV 16 (1.02) and subtracting the multiplied signal from the signal measured at 72° C. Second, the extracted signal for HPV 59 at 60° C. was obtained by multiplying the extracted signal for HPV 59 at 72° C. by the reference value for HPV 59 (1.21). Third, the extracted signal for HPV 16 at 60° C. was obtained by multiplying the signal measured at 83° C. by the reference value for HPV 16 (1.00). Fourth, the extracted signal for HPV 59 at 60° C. and the extracted signal for HPV 16 at 60° C. were subtracted from the signal measured at 60° C., thereby obtaining the extracted signal for HPV 51 at 60° C.

The extracted signal for HPV 33 at 60° C. for CAL Fluor Red 610 channel was obtained as follows:

First, the extracted signal for HPV 39 at 72° C. was obtained by multiplying the signal measured at 83° C. by the reference value for HPV 52 (1.05) and subtracting the multiplied signal from the signal measured at 72° C. Second, the extracted signal for HPV 39 at 60° C. was obtained by multiplying the extracted signal for HPV 39 at 72° C. by the reference value for HPV 39 (1.09). Third, the extracted signal for HPV 52 at 60° C. was obtained by multiplying the signal measured at 83° C. by the reference value for HPV 52 (0.94). Fourth, the extracted signal for HPV 39 at 60° C. and the extracted signal for HPV 52 at 60° C. were subtracted from the signal measured at 60° C., thereby obtaining the extracted signal for HPV 33 at 60° C.

The extracted signal for Internal Control at 60° C. for ATTO 647N channel was obtained as follows:

First, the extracted signal for HPV 35 at 72° C. was obtained by multiplying the signal measured at 83° C. by the reference value for HPV 18 (1.05) and subtracting the multiplied signal from the signal measured at 72° C. Second, the extracted signal for HPV 35 at 60° C. was obtained by multiplying the extracted signal for HPV 35 at 72° C. by the reference value for HPV 35 (1.03). Third, the extracted signal for HPV 18 at 60° C. was obtained by multiplying the signal measured at 83° C. by the reference value for HPV 18 (1.00). Fourth, the extracted signal for HPV 35 at 60° C. and the extracted signal for HPV 18 at 60° C. were subtracted from the signal measured at 60° C., thereby obtaining the extracted signal for Internal Control at 60° C.

The extracted signal for HPV 56 at 60° C. for ATTO 680 channel was obtained as follows:

First, the extracted signal for HPV 68 at 72° C. was obtained by multiplying the signal measured at 83° C. by the reference value for HPV 31 (1.11) and subtracting the multiplied signal from the signal measured at 72° C. Second, the extracted signal for HPV 68 at 60° C. was obtained by multiplying the extracted signal for HPV 68 at 72° C. by the reference value for HPV 68 (1.01). Third, the extracted signal for HPV 31 at 60° C. was obtained by multiplying the signal measured at 83° C. by the reference value for HPV 31 (1.00). Fourth, the extracted signal for HPV 68 at 60° C. and the extracted signal for HPV 31 at 60° C. were subtracted from the signal measured at 60° C., thereby obtaining the extracted signal for HPV 56 at 60° C.

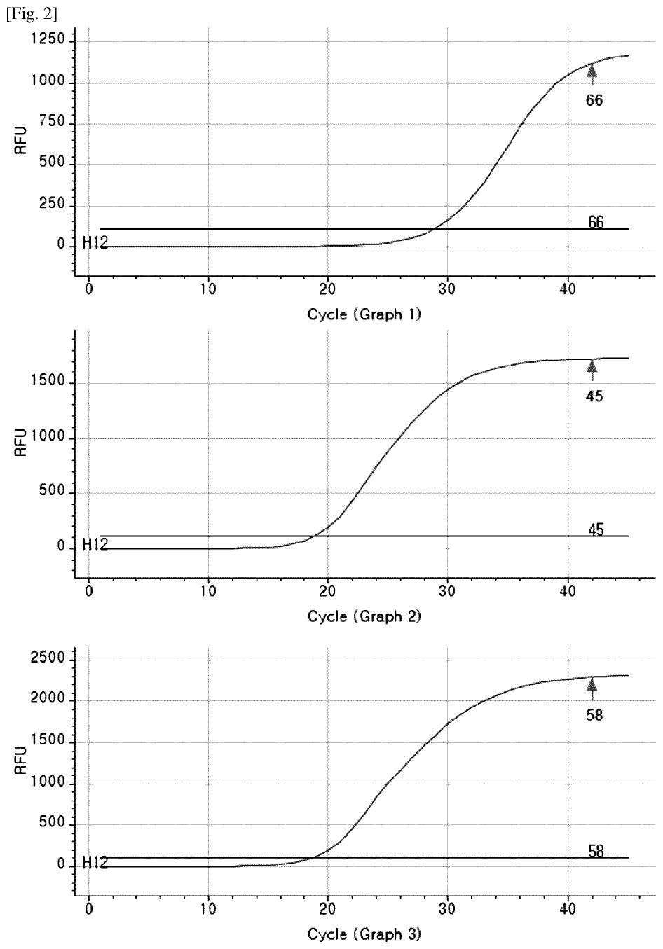

The extracted signal for each target nucleic acid for each detection channel through the above process is shown in FIGS. 2, 4, 6, 8, and 10. All RFU values were derived and exported from “Baseline subtracted curve fit” analysis data in instrumental software. Therefore, there are some differences between the signals of HPV 58, HPV 16, HPV 52, HPV 18, and HPV 31 shown in FIGS. 2, 4, 6, 8, and 10 and the signals measured at 83° C. shown in FIGS. 1, 3, 5, 7, and 9 due to fitting analysis, but they are substantially identical. In addition, to examine the significance of the extracted signals, a threshold of 110 was selected appropriately based on the results of each individual sample.

As shown in FIG. 2, the extracted amplification curve at 60° C. can indicate the presence or absence of HPV 66, which generates a signal only at 60° C., while the extracted amplification curve at 72° C. can indicate the presence or absence of HPV 45, which generates signals at 60° C. and 72° C. Further, the presence or absence of HPV 58, which generates signals at 60° C., 72° C. and 83° C. can be determined by examining the signal measured at 83° C.

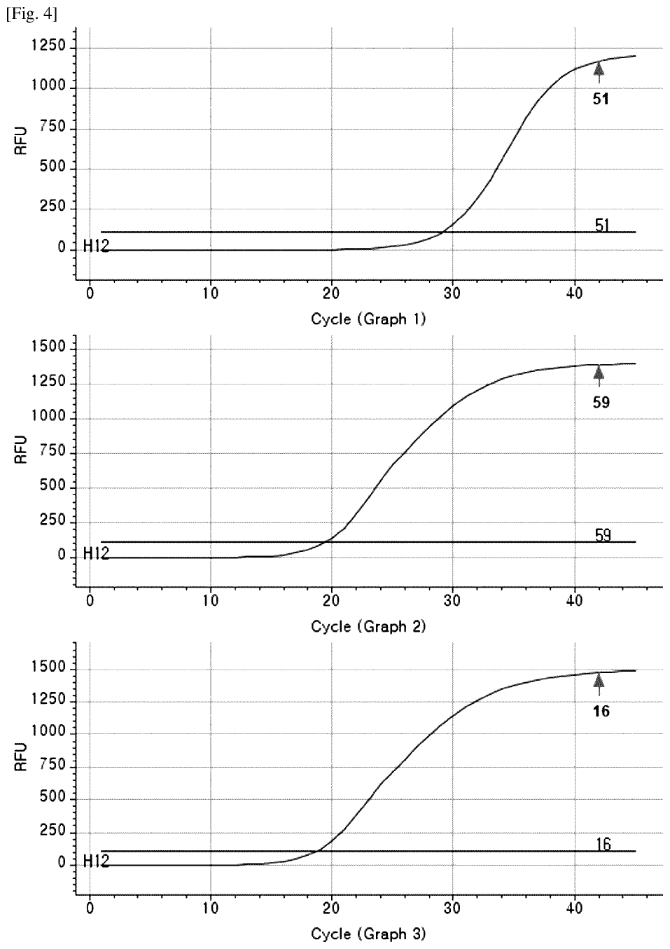

As shown in FIG. 4, the extracted amplification curve at 60° C. can indicate the presence or absence of HPV 51, which generates a signal only at 60° C., while the extracted amplification curve at 72° C. can indicate the presence or absence of HPV 59, which generates signals at 60° C. and 72° C. Further, the presence or absence of HPV 16, which generates signals at 60° C., 72° C. and 83° C. can be determined by examining the signal measured at 83° C.

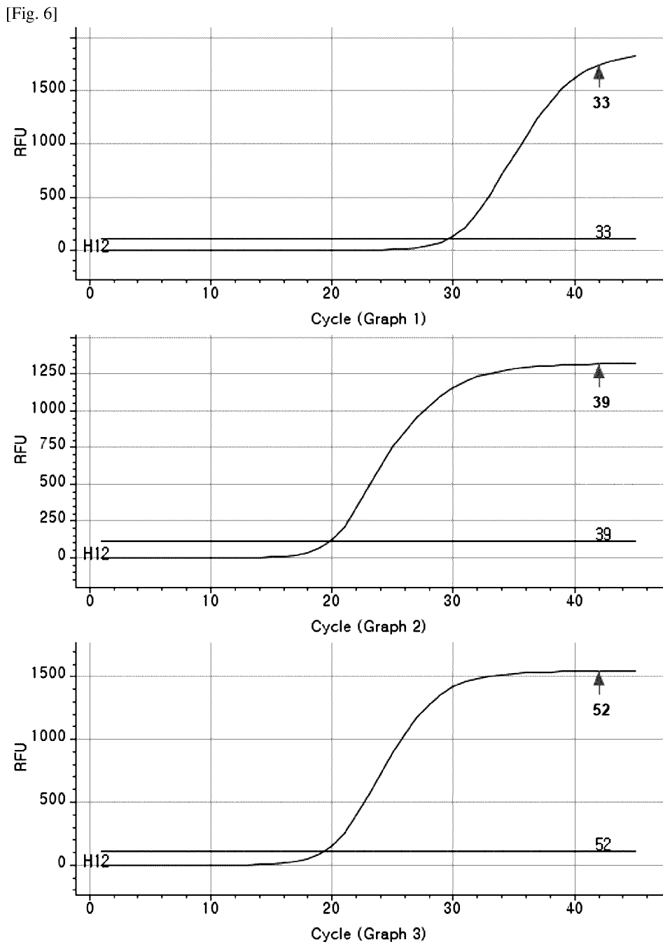

As shown in FIG. 6, the extracted amplification curve at 60° C. can indicate the presence or absence of HPV 33, which generates a signal only at 60° C., while the extracted amplification curve at 72° C. can indicate the presence or absence of HPV 39, which generates signals at 60° C. and 72° C. Further, the presence or absence of HPV 52, which generates signals at 60° C., 72° C. and 83° C. can be determined by examining the signal measured at 83° C.

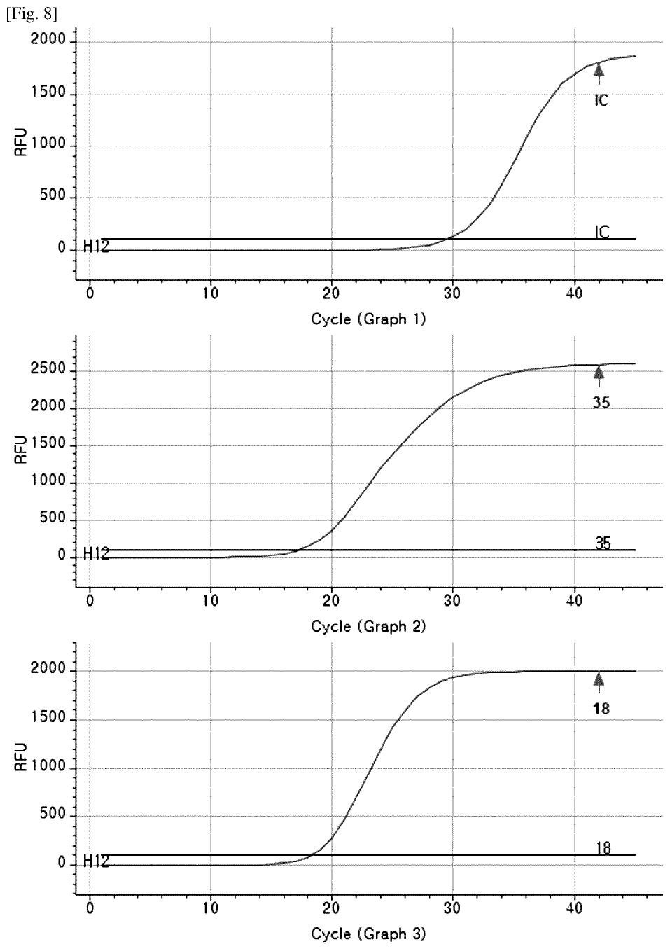

As shown in FIG. 8, the extracted amplification curve at 60° C. can indicate the presence or absence of IC, which generates a signal only at 60° C., while the extracted amplification curve at 72° C. can indicate the presence or absence of HPV 35, which generates signals at 60° C. and 72° C. Further, the presence or absence of HPV 18, which generates signals at 60° C., 72° C. and 83° C. can be determined by examining the signal measured at 83° C.

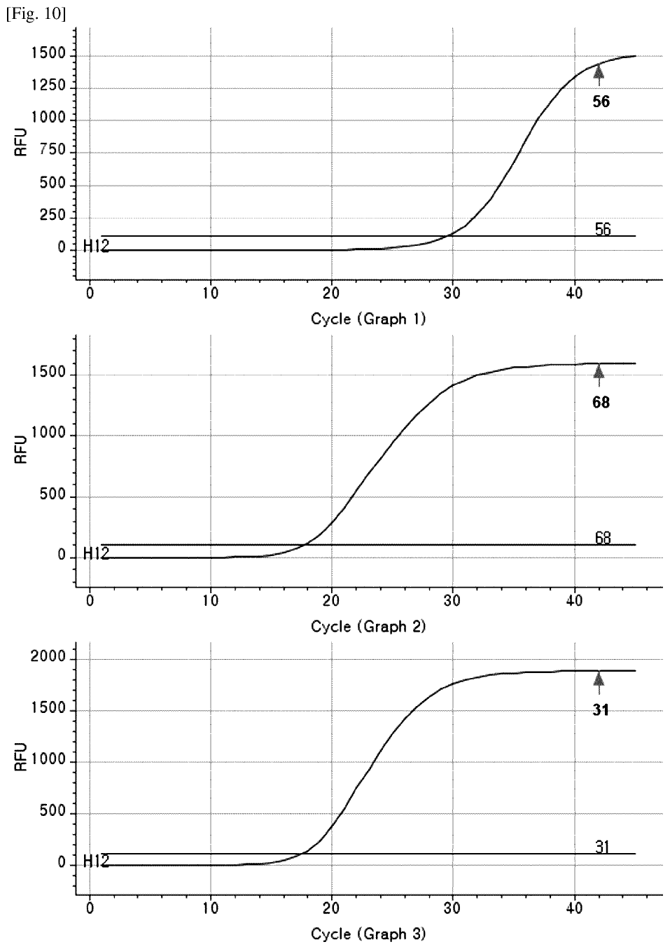

As shown in FIG. 10, the extracted amplification curve at 60° C. can indicate the presence or absence of HPV 56, which generates a signal only at 60° C., while the extracted amplification curve at 72° C. can indicate the presence or absence of HPV 68, which generates signals at 60° C. and 72° C. Further, the presence or absence of HPV 31, which generates signals at 60° C., 72° C. and 83° C. can be determined by examining the signal measured at 83° C.

The above results verify that the significant signals for target nucleic acids can be extracted or differentiated by using the signals measured at each detection temperature and the reference values. Therefore, three target nucleic acids can be detected by using real-time PCR with signal detections at different detection temperatures, single type of label in a single reaction vessel and a single type of detector. Further, it was confirmed that the presence of 15 targets (including internal control) can be determined in a more convenient and reliable manner by simultaneously analyzing five channels.

Having described a preferred embodiment of the present invention, it is to be understood that variants and modifications thereof falling within the spirit of the invention may become apparent to those skilled in this art, and the scope of this invention is to be determined by appended claims and their equivalents.

1. A method for multiplex detection of target nucleic acids of at least nine human papillomavirus (HPV) types in a sample, comprising:
(a) incubating the sample in a single reaction vessel with at least nine oligonucleotide sets to amplify or detect target nucleic acids of at least nine HPV types,
wherein each of the at least nine oligonucleotide sets comprises:
(i) an amplifying oligonucleotide, which serves to amplify a target nucleic acid of an HPV type of interest; and
(ii) a signaling oligonucleotide having a fluorescent label linked thereto, which serves to generate a signal in the presence of a target nucleic acid of an HPV type of interest,
wherein the amplifying oligonucleotide and the signaling oligonucleotide are identical to or different from each other,
wherein the fluorescent labels included in the at least nine oligonucleotide sets are of at least three different types, which are distinguishable by at least three different detection channels, and three of the at least nine oligonucleotide sets comprise any one of the at least three different types of the fluorescent labels,
wherein for three oligonucleotide sets comprising any one of the at least three different types of the fluorescent labels, one oligonucleotide set is designed to generate a signal at a first detection temperature in the presence of a target nucleic acid of a corresponding HPV type, another oligonucleotide set is designed to generate a signal at the first detection temperature and a second detection temperature in the presence of a target nucleic acid of another corresponding HPV type, and the other oligonucleotide set is designed to generate a signal at the first detection temperature, the second detection temperature and a third detection temperature in the presence of a target nucleic acid of the other corresponding HPV type;
(b) measuring signals at the first detection temperature, the second detection temperature, and the third detection temperature using each of the at least three detection channels; and
(c) determining the presence of target nucleic acids of the at least nine HPV types by the signals measured at the first detection temperature, the second detection temperature, and the third detection temperature using each of the at least three detection channels.

2. The method of claim 1, wherein the incubating is performed by realtime PCR in which Ct values are provided for each of target nucleic acids of the at least nine HPV types.

3. The method of claim 1, wherein the at least nine oligonucleotide sets comprise oligonucleotide sets specific for HPV types selected from the group consisting of HPV 16, 18, 31, 33, 35, 39, 45, 51, 52, 56, 58, 59, 66, and 68.

4. The method of claim 1, wherein the at least nine oligonucleotide sets comprise oligonucleotide sets specific for HPV types selected from the group consisting of HPV 6, 11, 26, 40, 42, 43, 44, 53, 54, 61, 69, 70, 73, and 82.

5. The method of claim 1, wherein the at least nine oligonucleotide sets are fifteen (15) oligonucleotide sets.

6. The method of claim 5, wherein the fifteen oligonucleotide sets consist of oligonucleotide sets specific for HPV 16, 18, 31, 33, 35, 39, 45, 51, 52, 56, 58, 59, 66, 68, and an internal control.

7. The method of claim 5, wherein the fifteen oligonucleotide sets consist of oligonucleotide sets specific for HPV 6, 11, 26, 40, 42, 43, 44, 53, 54, 61, 69, 70, 73, 82, and an internal control.

8. The method of claim 1, wherein the total number of oligonucleotides in the at least nine oligonucleotide sets is at least thirty-six (36).

9. The method of claim 1, wherein the total number of oligonucleotides in the at least nine oligonucleotide sets is at least forty-five (45).

10. The method of claim 1, wherein one or more of the at least nine oligonucleotide sets generate a signal by formation or dissociation of a duplex from the signaling oligonucleotide, which occurs dependently on the presence of a target nucleic acid.

11. The method of claim 1, wherein one or more of the at least nine oligonucleotide sets generate a signal by cleavage of a mediation oligonucleotide hybridized to the target nucleic acid and then formation or dissociation of a duplex between the cleavage product and the signaling oligonucleotide, which occurs dependently on the presence of the target nucleic acid.

12. The method of claim 1, wherein one or more of the at least nine oligonucleotide sets generate a signal by cleavage of the signaling oligonucleotide hybridized to the target nucleic acid and then dissociation of a duplex, which occurs dependently on the presence of the target nucleic acid.

13. The method of claim 1, wherein one or more of the at least nine oligonucleotide sets generate a signal by hybridization of the signaling oligonucleotide with the target nucleic acid and then cleavage, which occurs dependently on the presence of the target nucleic acid.

14. The method of claim 1, wherein the total concentration of the amplifying oligonucleotides in the reaction vessel is 15 to 60 pmole/μL.

15. The method of claim 1, wherein the total reaction volume in the reaction vessel is 10 to 30 μL.

16. The method of claim 1, wherein the total concentration of the signaling oligonucleotides in the reaction vessel is 8 to 35 pmole/μL.

17. The method of claim 1, wherein the first detection temperature is selected from 57 to 63° C., the second detection temperature is selected from 69 to 75° C., and the third detection temperature is selected from 80 to 86° C.

18. The method of claim 1, wherein the first detection temperature is selected from 80 to 86° C., the second detection temperature is selected from 69 to 75° C., and the third detection temperature is selected from 57 to 63° C.

19. The method of claim 1, wherein the step (c) is performed using signals measured at the first detection temperature, the second detection temperature, and the third detection temperature for each of the at least three detection channels, and three reference values.

20. The method of claim 1, wherein the step (c) comprises extracting three signals, a signal generated only by one oligonucleotide set, a signal generated only by another oligonucleotide set, and a signal generated only by the other oligonucleotide set, from signals measured at the first detection temperature, the second detection temperature and the third detection temperature for each of the at least three detection channels.

21.-25. (canceled)


SEQUENCE LISTING (60 sequences)

SEQ ID NO: 1 - DNA, 29 bp, synthetic construct
  source 1..29: mol_type = other DNA
ttttttcagatgtctctttggctgcctag

SEQ ID NO: 2 - DNA, 29 bp, synthetic construct
  source 1..29: mol_type = other DNA
ataggaaaatagggatgtccaactgcaag

SEQ ID NO: 3 - DNA, 34 bp, synthetic construct
  source 1..34: mol_type = other DNA
caccacagggttgaatatgttgcacgcacaaaca

SEQ ID NO: 4 - DNA, 38 bp, synthetic construct
  source 1..38: mol_type = other DNA
tttttttttataatatttactaatggaaccctgtggtg

SEQ ID NO: 5 - DNA, 32 bp, synthetic construct
  source 1..32: mol_type = other DNA
gggttacattaccatctactacctctgtatgg

SEQ ID NO: 6 - DNA, 31 bp, synthetic construct
  source 1..31: mol_type = other DNA
cttgtgcgagtcacataatcatcggtattta

SEQ ID NO: 7 - DNA, 37 bp, synthetic construct
  source 1..37: mol_type = other DNA
acagcctagcgttggctttgtggcggcctagtgacaa

SEQ ID NO: 8 - DNA, 38 bp, synthetic construct
  source 1..38: mol_type = other DNA
attatttttattattttttttattccacgctaggctgt

SEQ ID NO: 9-10 - skipped (2 entries)

SEQ ID NO: 11 - DNA, 46 bp, synthetic construct
  source 1..46: mol_type = other DNA
caccaaagtcggcctaaaaaaatagttgtaccaaaggtgtcaggat

SEQ ID NO: 12 - DNA, 38 bp, synthetic construct
  source 1..38: mol_type = other DNA
atttttttttttttatattataatggccgactttggtg

SEQ ID NO: 13 - DNA, 23 bp, synthetic construct
  source 1..23: mol_type = other DNA
tgaggccacagtgtacctgcctc

SEQ ID NO: 14 - DNA, 29 bp, synthetic construct
  source 1..29: mol_type = other DNA
tttttaatagaaaaatatggatggccaac

SEQ ID NO: 15 - DNA, 37 bp, synthetic construct
  source 1..37: mol_type = other DNA
ctccatggtgcatgtgtctcgcacaagcatttattat

SEQ ID NO: 16 - DNA, 38 bp, synthetic construct
  source 1..38: mol_type = other DNA
tattatttttatttattttatctgtctgcaccatggag

SEQ ID NO: 17-18 - skipped (2 entries)

SEQ ID NO: 19 - DNA, 37 bp, synthetic construct
  source 1..37: mol_type = other DNA
ccaatcgggactgggaaaaggcacaccttgtaatgct

SEQ ID NO: 20 - DNA, 38 bp, synthetic construct
  source 1..38: mol_type = other DNA
attattttttatttatttagtatgtcagtcccgattgg

SEQ ID NO: 21-22 - skipped (2 entries)

SEQ ID NO: 23 - DNA, 35 bp, synthetic construct
  source 1..35: mol_type = other DNA
aagctgacacccgtgtaggggtggaggtgggcagg

SEQ ID NO: 24 - DNA, 38 bp, synthetic construct
  source 1..38: mol_type = other DNA
attttttttattattatttatatcctgggtgtcagctt

SEQ ID NO: 25-26 - skipped (2 entries)

SEQ ID NO: 27 - DNA, 36 bp, synthetic construct
  source 1..36: mol_type = other DNA
aaatgwccgtcrcaggtttacaaagtgtgcccttgg

SEQ ID NO: 28 - DNA, 38 bp, synthetic construct
  source 1..38: mol_type = other DNA
tttttttttacactggctactctctgggacgggcattt

SEQ ID NO: 29-30 - skipped (2 entries)

SEQ ID NO: 31 - DNA, 37 bp, synthetic construct
  source 1..37: mol_type = other DNA
ggaacactccgtgggtattgccactacatgcaaaaac

SEQ ID NO: 32 - DNA, 38 bp, synthetic construct
  source 1..38: mol_type = other DNA
tttttttttttatttaatattaatggacggagtgttcc

SEQ ID NO: 33-34 - skipped (2 entries)

SEQ ID NO: 35 - DNA, 45 bp, synthetic construct
  source 1..45: mol_type = other DNA
aagcaaggacgctgcattttaggatgcaaacctcctataggtgaa

SEQ ID NO: 36 - DNA, 38 bp, synthetic construct
  source 1..38: mol_type = other DNA
attattttttttattatttatatcctgcgtccttgctt

SEQ ID NO: 37-38 - skipped (2 entries)

SEQ ID NO: 39 - DNA, 34 bp, synthetic construct
  source 1..34: mol_type = other DNA
ccacaagtctcgccggaccaggaacggttagtgt

SEQ ID NO: 40 - DNA, 38 bp, synthetic construct
  source 1..38: mol_type = other DNA
ttttttttttttataattattataggcgagacttgtgg

SEQ ID NO: 41-43 - skipped (3 entries)

SEQ ID NO: 44 - DNA, 38 bp, synthetic construct
  source 1..38: mol_type = other DNA
agcggtctcccagtcgttagtaggcaagcacaggcgtt

SEQ ID NO: 45-46 - skipped (2 entries)

SEQ ID NO: 47 - DNA, 38 bp, synthetic construct
  source 1..38: mol_type = other DNA
aatccctcggcactgcgtggtagaaaatactggtacgg

SEQ ID NO: 48 - DNA, 38 bp, synthetic construct
  source 1..38: mol_type = other DNA
tttttttttacagtgggtgatttcagtgccgagggatt

SEQ ID NO: 49-50 - skipped (2 entries)

SEQ ID NO: 51 - DNA, 38 bp, synthetic construct
  source 1..38: mol_type = other DNA
ccaagcagagaccccatacyaaacgttcctggtcagga

SEQ ID NO: 52 - DNA, 38 bp, synthetic construct
  source 1..38: mol_type = other DNA
tttttttttaattttaattattatgggtctctgcttgg

SEQ ID NO: 53-54 - skipped (2 entries)

SEQ ID NO: 55 - DNA, 32 bp, synthetic construct
  source 1..32: mol_type = other DNA
gtcccacctacacctgcttgcggcycccagac

SEQ ID NO: 56 - DNA, 38 bp, synthetic construct
  source 1..38: mol_type = other DNA
tttttttttaactttggtgctcgaggtgtaggtgggac

SEQ ID NO: 57-58 - skipped (2 entries)

SEQ ID NO: 59 - DNA, 42 bp, synthetic construct
  source 1..42: mol_type = other DNA
acgctgggcattagaagtaggagaaacatgcaaagtaaaagt

SEQ ID NO: 60 - DNA, 38 bp, synthetic construct
  source 1..38: mol_type = other DNA
attttattatttattatttttattccaatgcccagcgt